(19) United States
(12) Patent Application Publication
(10) Pub. No.: US 2005/0096304 A1
(43) Pub. Date: May 5, 2005

(54) Method of treating cancer using dithiocarbamate derivatives

(76) Inventors: David White, Chicago, IL (US); Robert R. Whittle, Wilmington, NC (US); Grayson Walker Stowell, Wilmington, NC (US); Linda B. Whittall, Wilmington, NC (US); Thomas Kennedy, Charlotte, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/922,728

(22) Filed: Aug. 20, 2004

Related U.S. Application Data
(63) Continuation-in-part of application No. 10/378,206, filed on Mar. 3, 2003.
Application No. 10/378,206 is a division of application No. 09/735,205, filed on Dec. 12, 2000, now Pat. No. 6,548,540.
Application No. 09/735,205 is a continuation-in-part of application No. 09/679,932, filed on Oct. 5, 2000, now Pat. No. 6,706,759.
Application No. 09/679,932 is a continuation-in-part of application No. 09/392,122, filed on Sep. 8, 1999, now Pat. No. 6,589,987.
(60) Provisional application No. 60/099,390, filed on Sep. 8, 1998.

(51) Int. Cl. A61K 31/555; A61K 31/27

(52) U.S. Cl. 514/184; 514/492

(57) ABSTRACT

The invention encompasses neutral dithiocarbamate metal compounds and methods of treating cancer using such compounds, along with methods for sensitizing AIDS/HIV patients to anti-retroviral therapy by blocking the P-glycoprotein membrane toxin extrusion pump using such compounds. Compounds inhibit the growth of cancer cells of a variety of cell types. A method is presented for using the neutral compounds disclosed herein, amongst other uses disclosed herein, to reduce tumor growth, and to potentiate the effect of other anticancer agents. The invention also encompasses pharmaceutical compositions comprising the neutral compounds and a pharmaceutically acceptable excipient, diluent, solubilizer, solvent, adjuvant or carrier, or a mixture thereof.

### FIELD OF INVENTION

This invention generally relates to neutral, metallic dithiocarbamate compounds and methods of treating cancer, and particularly to methods of treating cancer using such metallic dithiocarbamate compounds. Also encompassed in the invention is a method of sensitizing AIDS/HIV patients to anti-retroviral therapy using neutral, metallic dithiocarbamate metal compounds.

### BACKGROUND OF THE INVENTION

Cancer, the uncontrolled growth of malignant cells, is a major health problem of the modern medical era and ranks second only to heart disease as a cause of death in the United States. While some malignancies, such as adenocarcinoma of the breast and lymphomas such as Hodgkin's disease, respond relatively well to current chemotherapeutic antineoplastic drug regimens, other cancers respond poorly to chemotherapy. Among those cancers that respond least well to chemotherapy are non-small cell lung cancer, pancreatic, prostate, and colon cancers. Even small cell cancer of the lung, initially chemotherapy sensitive, tends to return after remission with extensive metastatic spread leading to the death of the patient. Thus, better treatment approaches are needed for these illnesses. Almost all of the currently available antineoplastic agents have limited applicability in patients, as they impart significant toxicities to the human patient, such as bone marrow suppression, renal dysfunction, stomatitis, enteritis and hair loss.

The end of the twentieth century has seen a dramatic increase in the observed incidence of malignant melanoma than all other types of tumors. The biology of malignant melanomas offers an example of the importance of transcription factors for malignant cell propagation. Malignant melanomas have great propensity to metastasize and are notoriously resistant to conventional cancer treatments such as chemotherapy and y-irradiation. Without being bound by any particular theory, it is believed that development of malignant melanoma in humans progresses through a multistage process, with transition from melanocyte to nevi, to radial growth, and subsequently to the vertical growth, metastatic phenotype of autonomous melanomas associated with decreased dependence on growth factors, diminished anchorage dependence, reduced contact inhibition, and increased radiation and drug resistance.

Much of the molecular understanding of melanoma progression has come from studying the response of cultured melanoma cells to mitogenic stimuli. In culture, melanocyte proliferation and differentiation are positively regulated by agents that increase cAMP (See e.g., P. M. Cox, et al., “An ATF/CREB binding motif is required for aberrant constitutive expression of the MHC class II Drα promoter and activation by SV40 T-antigen,” Nucleic Acids Res. 20:4881-4887 (1992); R. Halaban, et al., “Regulation of tyrosinase in human melanocytes grown in culture,” J. Cell Biol. 97:480-488 (1983); D. Jean, et al., “CREB and its associated proteins act as survival factors for human melanoma cells,” J. Biol. Chem. 273:24884-24890 (1998); P. Klatt, et al., “Nitric oxide inhibits c-Jun DNA binding by specifically targeted S-glutathionylation,” J. Biol. Chem. 274:15857-15864 (1999); J. M. Lehmann, et. al., “MUC18, a marker of tumor progression in human melanoma, shows sequence similarity to the neural cell adhesion molecules of the immunoglobulin superfamily,” Proc. Natl. Acad. Sci. U.S.A. 89:9891-9895 (1989); M. Luca, et al., “Direct correlation between MUC18 expression and metastatic potential of human melanoma cells,” Melanoma Res. 3:35-41(1993); J. P. Richards, et al., “Analysis of the structural properties of cAMP-responsive element-binding protein (CREB) and phosphorylated CREB,” J. Biol. Chem. 271:13716-13723 (1996); and S. Xie, et al., “Dominant-negative CREB inhibits tumor growth and metastasis of human melanoma cells,” Oncogene 15:2069-2075 (1997)), and several cAMP responsive transcription factors binding to CRE (the consensus motif 5′-TGACGTCA-3′, or cAMP response element) play prominent roles in mediating melanoma growth and metastasis. In MeWo melanoma cells, the transcription factor CREB (for CRE-binding protein) and its associated family member ATF-1 promote tumor growth, metastases and survival through CRE-dependent gene expression. (See D. Jean, et al., supra). Expression of the dominant negative KCREB construct in metastatic MeWo melanoma cells decreases their tumorigenicity and metastatic potential in nude mice. (See S. Xie, et al., “Expression of MCA/MUC18 by human melanoma cells leads to increased tumor growth and metastasis,” Cancer Res. 57:2295-2303 (1997)). The KCREB-transfected cells display a significant decrease in matrix metalloproteinase 2 (MPP2, the 72 kDa collagenase type IV) mRNA and activity, resulting in decreased invasiveness through the basement membrane, an important component of metastatic potential.

The cell surface adhesion molecule MCAM/MUC18, which is involved in metastasis of melanoma (See J. M. Lehmann, et al., supra; M. Luca, et al., supra; S. Xie, et al., supra), is also down-regulated by KCREB transfection. (See S. Xie, et al., Cancer Res., supra). In addition, expression of KCREB in MeWo cells renders them susceptible to thapsigargin-induced apoptosis, suggesting that CREB and its associated proteins act as survival factors for human melanoma cells, thereby contributing to the acquisition of the malignant phenotype. (See D. Jean, et al., supra).

Melanoma cells aberrantly express the major histocompatibility complex class II (MHC II) antigens, normally found only in B-lymphocytes and antigen presenting cells of the monocyte/macrophage cell line. (See P. M. Cox, et al., “An ATF/CREB binding motif is required for aberrant constitutive expression of the MHC class II Drα promoter and activation by SV40 T-antigen. Nucleic Acids Res.,” 20:4881-4887 (1992)). In B₁₆ melanoma cells this is due to activation of the MHC II DRα promoter by constitutive activation of an ATF/CREB motif. CREB family proteins also bind to the UV-response element (URE, 5′-TGACAACA-3′), and URE binding of the CREB family member ATF2 confers resistance to irradiation and to the chemotherapeutic drugs cisplatin, 1-β-D-arabinofuranosylcytosine (araC), or mitomycin C in MeWo melanoma lines. (See Z. Ronai, et al., “ATF2 confers radiation resistance to human melanoma cells,” Oncogene 16:523-531 (1998)). Thus, it is believed that the CREB family transcription factors play important roles in the malignant potential of this important tumor type. This has led to the suggestion by others that targeted molecular disruption of ATF/CREB-mediated transcription might be therapeutically useful for controlling growth and metastases of relatively treatment-resistant malignant melanoma. (See D. Jean, supra, and Z. Ronai, supra).

The positively charged DNA binding domain of many transcription factors contains cysteines, which can be oxidatively modified by radicals such as hydroxyl (HO.) or nitric oxide (NO.), stimulating repair processes that result in formation of mixed disulfides between glutathione (GSH) and protein thiols. (See P. Klatt, et al., supra; and H. Sies, “Glutathione and its role in cellular functions,” Free Rad. Biol. Med. 27:916-921 (1999)). As a consequence of this so-called protein “S-glutathionylation”, the usually positively charged transcription factor DNA binding domain develops a negative charge imparted by dual carboxylate end groups of GSH. The change in charge disrupts transcription factor binding to its respective DNA consensus sequence. (See P. Klatt, et al., supra and H. Sies, supra). This mechanism has been demonstrated to explain how NO inhibits c-Jun DNA binding by specifically targeted S-glutathionylation of cysteines within the DNA binding region, and a similar mechanism has been suggested for how nitrosative stress in general might functionally inhibit the activity of Fos, ATF/CREB, Myb, and Rel/NFκB family transcription factors. (See P. Klatt, et al., supra).

Dithiocarbamates are a broad class of molecules that have the ability to chelate to metal ions, as well as to react with sulfhydryl groups and glutathione. After metal-mediated conversion to their corresponding disulfides, dithiocarbamates inhibit cysteine proteases by forming mixed disulfides with critical protein thiols. (See C. S. I. Nobel, et al., “Mechanism of dithiocarbamate inhibition of apoptosis: thiol oxidation by dithiocarbamate disulfides directly inhibits processing of the caspase-3 proenzyme,” Chem. Res. Toxicol. 10:636-643 (1997)). CREB contains three cysteines in the DNA binding region (Cys³⁰⁰, Cys³¹⁰ and Cys³³⁷), which are not essential for DNA binding but might provide reactive sites for S-glutathionylation. (See S. Orrenius, et al., “Dithiocarbamates and the redox regulation of cell death,” Biochem. Soc. Trans. 24:1032-1038 (1996)).

Recently, dithiocarbamates containing a reduced sulfhydryl group, e.g., pyrrolidinedithiocarbamate PDTC, have been shown to inhibit the proliferation of cultured colorectal cancer cells. (See Chinery, et al., “Antioxidants enhance the cytotoxicity of chemotherapeutic agents in colorectal cancer: a p53-independent induction of p21^{WAFI/CIPI} via C/EBPβ,” Nature Med. 3:1233-1241 (1997); Chinery et al., “Antioxidants reduce cyclooxygenase-2 expression, prostaglandin production, and proliferation in colorectal cancer cells.” Cancer Res. 58:2323-2327 (1998)).

In addition to their reduced thioacid form, dithiocarbamates can also or are known to exist in four other forms: a) the disulfide, a condensed dimer of the thioacid with elimination of reduced sulfhydryl groups by disulfide bond formation; b) the negatively charged thiolate anion, generally as a salt, such as the sodium salt or ammonium salt; c) the 1,1-dithiolato coordination complex of metal ions in which the two adjoining sulfur atoms of the dithiocarbamate are bound to the same metal ion, for example, titanium(III), vanadium(III), chromium(III), iron(III), cobalt(III), nickel(II), copper(II), silver(I), gold(III), Zn(II), Au(I), Mn(III), Ga(III), Pt(II); and d) the monodentate dithiolato coordination complex in which either one of the sulfur atoms binds to a metal ion, for example titanium(III), vanadium(III), chromium(III), iron(III), cobalt(III), nickel(II), copper(II), silver(I), or gold(III). The disulfide, thiolate anion, and coordination complexes of dithiocarbamates are all structurally distinct from the reduced form of PDTC used by Chinery, et al., in that they have no reduced sulfhydryl molecular moiety and are incapable of functioning as antioxidants by donating the proton from a reduced sulfhydryl to scavenge electrons of free radical species. Without being bound by any particular theory, in lacking a reduced sulfhydryl, dithiocarbamate disulfides, thiolate anions, and coordination complexes should, according to the teachings of Chinery, et al., have no activity as antiproliferative compounds against cancer since these three non-reduced chemical forms of dithiocarbamates are incapable of functioning as antioxidants.

In U.S. patent application Ser. No. 09/392,122; filed Sep. 8, 1999, it was reported that the dithiocarbamate disulfide disulfiram sensitizes tumor cells to cancer chemotherapy and could be used in conjunction with cancer chemotherapeutic drugs to increase their effectiveness in treating neoplasms. Recently, this effect has been explained in work in which disulfiram was shown to prevent maturation of the P-glycoprotein pump, an ATP-driven 170-kd efflux pump on the plasma membrane that pumps a variety of cytotoxic drugs out of cells. (See T. W. Loo, et al., “Blockage of drug resistance in vitro by disulfiram, a drug used to treat alcoholism.” J. Natl. Cancer Inst. 92:898-902 (2000)). This effect reduces P-glycoprotein-mediated drug resistance in tumor cells and sensitizes tumor cells to cancer chemotherapy.

Without being bound any particular theory, sensitization of cancer cells to chemotherapy is thought to be provided by the ability of dithiocarbamates to block nuclear factor-κB (NF-κB), which is constitutively activated in many malignancies and upregulates expression of anti-apoptotic factors (Baldwin A S. Control of oncogenesis and cancer therapy resistance by the transcription factor NF-κB. J Clin Invest 107:241-246, 2001). Blocking NF-κB with disulfiram sensitizes colon cancer cells to 5-FU (Wang W, McLeod H L, Cassidy J. Disulfiram-mediated inhibition of NF-κB activity enhances cytotoxicity of 5-fluorouracil in colorectal cancer cell lines. Int J Cancer 104:504-511, 2003).

In addition to the foregoing, it is known in the art that the dithiocarbamate alcoholism drug disulfiram blocks the P-glycoprotein extrusion pump, inhibits the transcription factor nuclear factor κB (NF-κB), sensitizes tumors to chemotherapy, reduces angiogenesis and inhibits tumor growth in mice. Dithiocarbamates are also known to react with critical thiols and also complex metal ions. It has been surprisingly found that disulfiram administered to melanoma cells in combination with copper(II) or zinc(II) decreased expression of cyclin A and reduces proliferation in vitro at lower concentrations than disulfiram alone. It has also been surprisingly discovered that in electrophoretic mobility shift assays, disulfiram decreases transcription factor binding to the cyclic-AMP response element CRE in a manner potentiated by copper(II) ions and by the presence of glutathione. Without being bound by any particular theory, dithiocarbamates are believed to disrupt transcription factor binding by inducing S-glutathionylation of the transcription factor DNA binding region. It has been surprisingly found that disulfiram inhibits growth and angiogenesis in melanomas transplanted in SCID mice, and these effects are potentiated by zinc(II) supplementation. We have also surprisingly found that the combination of oral zinc gluconate and disulfiram at currently approved doses for alcoholism also induces greater than 50% reduction in hepatic metastases and produces clinical remission in a patient with Stage IV metastatic ocular melanoma, where such patient has continued on oral zinc gluconate and disulfiram therapy for 53 continuous months with negligible side effects. These surprising findings present a novel strategy for treating metastatic melanoma by employing metal complexes for new therapeutic uses.

Recently, a number of laboratories have investigated the aldehyde dehydrogenase inhibitor tetraethylthiuram disulfide, or disulfiram, a relatively nontoxic (oral LD₅₀ of 8.6 g/kg; see, Budavari S, editor. Merck Index. 12^{th} ed. Whitehouse Station (NJ): Merck Research Laboratories, 1996) dithiocarbamate disulfide long used for alcohol aversion therapy. See, Johansson B. A review of the pharmacokinetics and pharmacodynamics of disulfiram and its metabolites. Acta Psychitrica Scand, Suppl, 1992; 369:15-26. It is known in the art that disulfiram reverses in vitro resistance of human tumors to chemotherapy drugs by blocking maturation of the P-glycoprotein membrane pump that extrudes chemotherapeutic agents from the cell. See, Loo T W, Clarke D M. Blockage of drug resistance in vitro by disulfiram, a drug used to treat alcoholism. J Natl Cancer Inst, 2000; 92:898-902. It is also know in the art that disulfiram also inhibits activation of nuclear factor-κB (NF-κB) induced in human colorectal cancer cell lines by the chemotherapeutic agent 5-fluorouracil (5-FU), and enhances the apoptotic effect 5-FU in vitro when the two are used in combination. See, Wang W, McLeod H L, Cassidy J. Disulfiram-mediated inhibition of NF-κB activity enhances cytotoxicity of 5-fluorouracil in colorectal cancer cell lines. Int J Cancer, 2003;104:504-511. Additionally, it is known to those of skill in the art that disulfiram inhibits DNA topoisomerases (see, Yakisch J S, Siden A, Eneroth P, et al. Disulfiram is a potent in vitro inhibitor of DNA topoisomerases. Biochem Biophys Res Commun, 2002; 289:586-590), induces apoptosis in cultured melanoma cells (see, Cen D, Gonzalez R I, Buckmeir J A, et al. Disulfiram induces apoptosis in human melanoma cells: a redox-related process. Mol Cancer Therapeut, 2002; 1:197-204), reduces angiogenesis (see, Shiah S-G, Kao Y R, Wu F, et al. Inhibition of invasion and angiogenesis by zinc-chelating agent disulfiram. Mol Pharmacol, 2003; 64:1076-1084; and Marikovsky M, Ziv V, Nevo M, et al. Cu/Zn superoxide dismutase plays important role in immune response. J Immunol, 2003; 170:2993-3001), inhibits matrix metalloproteinases and cancer cell invasiveness (see, Shiah S-G, Kao Y R, Wu F, et al. Inhibition of invasion and angiogenesis by zinc-chelating agent disulfiram. Mol Pharmacol, 2003; 64:1076-1084), and retards growth of C6 glioma and Lewis lung carcinoma in mice. See, Marikovsky M, Nevo N, Vadai E, et al. Cu/Zn superoxide dismutase plays a role in angiogenesis. Int J Cancer, 2002; 97:34-41. However, the mechanism for disulfiram's effects is still not clear, and the use of disulfiram is yet to be reported in the treatment of human malignancies.

The anti-neoplastic activity of disulfiram has been attributed in the art to pro-apoptotic redox-related mitochondrial membrane permeabilization (see, Cen D, Gonzalez R I, Buckmeir J A, et al. Disulfiram induces apoptosis in human melanoma cells: a redox-related process. Mol Cancer Therapeut, 2002; 1:197-204), zinc complexation, with subsequent inhibition of Zn(II)-dependent matrix metalloproteinases (see, Shiah S-G, Kao Y R, Wu F, et al. Inhibition of invasion and angiogenesis by zinc-chelating agent disulfiram. Mol Pharmacol, 2003; 64:1076-1084), or Cu(II) complexation, with inactivation of Cu/Zn superoxide dismutase (SOD) (see, Marikovsky M, Ziv V, Nevo M, et al. Cu/Zn superoxide dismutase plays important role in immune response. J Immunol, 2003; 170:2993-3001; and Marikovsky M, Nevo N, Vadai E, et al. Cu/Zn superoxide dismutase plays a role in angiogenesis. Int J Cancer, 2002; 97:34-41) and consequently diminished cellular generation of H₂O₂ from dismutation of superoxide anion (O₂⁻). See, Marikovsky M, Ziv V, Nevo M, et al. Cu/Zn superoxide dismutase plays important role in immune response. J Immunol, 2003; 170:2993-3001; and Marikovsky M, Nevo N, Vadai E, et al. Cu/Zn superoxide dismutase plays a role in angiogenesis. Int J Cancer, 2002; 97:34-41). It is known that dithiocarbamates possess a R¹R²NC(S)S (wherein R¹ and R² are defined herein) functional group, giving them the ability to complex metals and react with sulfhydryl groups (see, Nobel C S I, Kimland M, Lind B, et al. Dithiocarbamates induce apoptosis in thymocytes by raising the intracellular level of redox-active copper. J Biol Chem, 1995; 270:26202-26208) and glutathione. See, Burkitt M J, Bishop H S, Milne L, et al. Dithiocarbamate toxicity toward thymocytes involves their copper-catalyzed conversion to thiuram disulfides, which oxidize glutathione in a redox cycle without the release of reactive oxygen species. Arch Biochem Biophys, 1998; 353:73-84. Without being bound by any particular theory, it is believed that after oxidation to their corresponding disulfides, dithiocarbamates can inhibit critical sulfhydryls by forming mixed disulfides with critical cellular thiols (see, Nobel C S I, Burgess D H, Zhivotovsky B, et al. Mechanism of dithiocarbamate inhibition of apoptosis: thiol oxidation by dithiocarbamate disulfides directly inhibits processing of the caspase-3 proenzyme. Chem Res Toxicol, 1997; 10:636-643), leading to such diverse effects as inhibition of caspases (see, Nobel C S I, Burgess D H, Zhivotovsky B, et al. Mechanism of dithiocarbamate inhibition of apoptosis: thiol oxidation by dithiocarbamate disulfides directly inhibits processing of the caspase-3 proenzyme. Chem Res Toxicol, 1997; 10:636-643), but stimulation of mitochondrial permeability transition (see, Balakirev M Y, Zimmer G. Mitochondrial injury by disulfiram: two different mechanisms of the mitochondrial permeability transition. Chem-Biol Interact, 2001; 1138:299-311) and subsequent Bcl-independent apoptosis. See, Constantini P, Belzacq A-S, Vieira H L A, et al. Oxidation of a critical thiol residue of the adenine nucleotide translocator enforces Bcl-2-independent permeability transition pore opening and apoptosis. Oncogene, 2000; 19:307-314. It is further known that in normal cells, the effects of other dithiocarbamates are potentiated by metals such as Cu(II) or Zn(II). See, Erl W, Weber C, Hansson G K. Pyrrolidine dithiocarbamate-induced apoptosis depends on cell type, density, and the presence of Cu(II) and Zn(II). Am J Physiol Cell Physiol, 2000; 278:C116-C1125.

The invention provides a treatment of malignant melanoma, a tumor notoriously resistant to radiation and traditional chemotherapeutic agents, but independently sensitive in vitro to disulfiram (see, e.g., Cen D, Gonzalez R I, Buckmeir J A, et al. Disulfiram induces apoptosis in human melanoma cells: a redox-related process. Mol Cancer Therapeut, 2002; 1:197-204) or metals (see, e.g., Borovansky J, Blasko M, Siracky J, et al. Cytotoxic interactions of Zn(II) in vitro: melanoma cells are more susceptible than melanocytes. Melanoma Res, 1997; 7:449-453). We have surprisingly discovered that disulfiram reduces ATF/CREB transcription factor DNA binding, cyclin A expression, cell cycle progression, and melanoma proliferation in vitro and in SCID mice in a manner dependent upon and facilitated by copper and other heavy metal ions. In addition, we have discovered a useful therapy, wherein a patient with Stage IV ocular melanoma and hepatic metastases, experiences considerable tumor regression and remains clinically well after 49 continuous months of therapy with oral disulfiram and zinc gluconate.

We have surprisingly discovered that disulfiram reduces cyclin A expression, cell cycle progression into G₂-M and melanoma proliferation in vitro in a manner both dependent upon and facilitated by heavy metal ions. We have also surprisingly discovered, without being bound by any particular theory, in the presence of heavy metals ions, disulfiram also substantially inhibits growth of human melanomas in SCID mice and reduces angiogenesis in the implanted tumors. We have surprisingly found that when disulfiram and zinc gluconate are co-administered to a patient with Stage IV metastatic ocular melanoma, the subject experiences impressive resolution of hepatic metastases with minimal side effects. We have also found that in the absence of any other concurrent therapy for a patient's tumor, the patient remains alive and clinically well with radiographically stable disease after 53 continuous months of disulfiram and zinc(II) therapy.

Employed at the currently approved dose of 250 mg daily, disulfiram appears safe and is readily available for application to a number of novel treatment strategies for malignancies.

The invention provides neutral dithiocarbamate metal compounds and improved methods for the treatment of cancer, and other indications disclosed hereunder, utilizing such compounds.

The invention further provides pharmaceutical compositions comprising neutral dithiocarbamate metal compounds useful for the treatment of cancer and other indications as disclosed herein.

The invention also provides methods employing neutral dithiocarbamate metal compounds for sensitizing AIDS/HIV patients to anti-retroviral therapy. Without being bound by any particular theory, such method of sensitization is believed to involve the blocking the P-glycoprotein membrane toxin extrusion pump.

The invention also provides relatively low-toxicity neutral dithiocarbamate metal compounds, for use alone or in combination with known cancer treatment agents, in order to more efficaciously treat cancer patients minimizing risking injury to said patient from the therapy itself.

The invention provides pharmaceutical compositions comprising a neutral dithiocarbamate metal compound and at least one pharmaceutically acceptable excipient, diluent, solubilizer, solvent, adjuvant, carrier or a mixture thereof.

The invention also provides the use of a neutral dithiocarbamate metal compound for the manufacture of a medicament.

### SUMMARY OF THE INVENTION

The invention encompasses the neutral dithiocarbamate metal compounds of formula (I) shown below, pharmaceutical compositions containing the compounds, unit dosage forms, and methods employing such compounds, compositions or forms in the treatment of cancer and for sensitizing AIDS/HIV patients to anti-retroviral therapy.

In a broad aspect, the invention provides a neutral compound of formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different; and
wherein each A, B and C may be the same or different.

Compounds of formula (I) may exist in a variety of forms, including for example but not limited to, complexes, salts, ion pairs, organometallics, and the like.

Without being bound by any particular theory, the oxidation state of the metal ion governs the number of ligands surrounding the metal ion such that a neutral coordination compound is formed, irregardless of the binding mode the dithiocarbamate ligand always carries a charge of −1.

In a further broad aspect, the invention includes all isomers (cis/trans; mer/fac; axial/equatorial; enantiomers; diasteriomers; Λ; Δ; δ; λ; etc.), solvates, polymorphs, hydrates, isotopically labeled derivatives, and metabolites, and mixtures thereof, of compounds of formula (I).

The invention provides methods for treating cancer using neutral dithiocarbamate metal compounds either alone or in combination with other therapeutically effective anti-cancer agents. Generally, the method encompasses administration of a therapeutically effective amount of such compounds to a patient in need thereof. Typically the method is applicable to animals, where preferably the patient is a mammal, more preferably a human, who has been diagnosed with cancer. The invention also provides a method for sensitizing AIDS/HIV patients to anti-retroviral therapy by blocking the P-glycoprotein membrane toxin extrusion pump using neutral dithiocarbamate metal compounds either alone or in combination with other therapeutically effective compounds for such purpose. Without being bound by any particular theory, such sensitization occurs via blockage of the P-glycoprotein membrane toxin extrusion pump.

It has been discovered that neutral dithiocarbamate metal compounds exhibit potent inhibitory effects on growth of established tumor cells in the absence of antioxidant sulfhydryl groups within their structure. Neutral dithiocarbamate metal compounds are effective in inhibiting the growth of established melanomas and non-small cell lung cancer cells, which are known to be poorly responsive to currently available neoplastic agents. In addition, it has further been surprisingly discovered that the antiproliferative and antineoplastic effect of neutral dithiocarbamate metal compounds on established tumor cells is greatly potentiated by co-treatment of cancer cells with a transition metal ion supplement in a concentration that, by itself, does not impair cancer cell growth. The potentiating function of the metal ion is to facilitate formation of the thiolate anion from the dithiocarbamate disulfide. Further, the tumor cell growth inhibition effect can be significantly enhanced by the addition of metal ions such as, but not limited to, copper(II), zinc(II), gold(III), and silver(I), as examples, or by administering the dithiocarbamate as a coordination compound.

Without being bound by any particular theory, it is believed that the chemical activity of these metal dithiocarbamate species is not from antioxidant action but from stimulating formation of mixed disulfides between the dithiocarbamate and sulfhydryl moieties of cysteines located at critical sites on cell proteins, such as the DNA binding region of transcription factors needed to promote expression of gene products necessary for malignant cell proliferation.

Dithiocarbamate disulfides, which are useful in the treatment of cancer or the sensitization of AIDS/HIV patients include, but are not limited to, those of the formula (II): 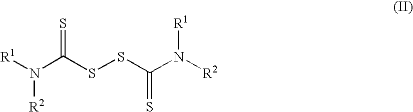
wherein each R¹ and R², at each occurrence, are independently as defined herein, i.e., the dithiocarbamate disulfides may be symmetrical or asymmetrical. In a preferred embodiment, R¹ and R² at each occurrence are independently hydrogen, C₁₋₁₂ alkyl, C₂₋₁₂ alkenyl, C₂₋₁₂ alkynyl, C₃₋₈ cycloalkyl, C₅₋₈ cycloalkenyl, C₅₋₈ cycloalkynyl, heterocyclyl, heterocycloalkyl, aryl or heteroaryl. The two substituents on any or both nitrogens may be incorporated into a saturated or unsaturated heterocyclic ring, i.e., each R¹ and R² attached to the same nitrogen may form a ring structure, which may include the nitrogen to which they are attached. Typically, R¹ and R² are not both hydrogen.

In another preferred embodiment, the neutral dithiocarbamate metal compound is administered in combination with another anticancer agent. In addition, the present invention provides methods for sensitizing cancer cells to chemotherapeutic drugs by the administration of a neutral dithiocarbamate metal compound in order to effect inhibition of the tumor cell membrane P-glycoprotein pump which functions to extrude from cancer cells the anti-neoplastic agents that are absorbed.

The invention provides pharmaceutical formulations that comprise at least one neutral compound of formula (I) and a pharmaceutically acceptable excipient, diluent, solubilizer, solvent, adjuvant, carrier or a mixture thereof. Optionally, the formulation can further contain another anticancer agent.

The active compounds of this invention can be administered through a variety of different routes. For example, they can be administered orally, intravenously, intradermally, subcutaneously, or topically.

The invention includes methods of treating various types of cancer, including but not limited to melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. In particular the present invention will be especially effective in treating melanoma, lung cancer, breast cancer, colon cancer and prostate cancer. Thus, the use of neutral dithiocarbamate metal compounds in this invention offers a readily available and easily used treatment for cancers in humans and other mammals.

The invention provides methods of removing existing multi-drug resistance or of avoiding the development of multi-drug resistance in an animal in need of such treatment, which methods comprise the treatment of an animal wth at least one neutral compound of formula (I) or a pharmaceutical formulation comprising at least one neutral compound of formula (I).

### BRIEF DESCRIPTION OF THE DRAWINGS

FIG. 1, shows disulfiram inhibition of the proliferation of CRL1619 human melanoma cells.

FIG. 2, shows disulfiram induction of apoptosis in melanoma measured by 3′-OH fluorescein end-labeling of DNA fragments (2A: CRL1619 melanoma cells treated with DMSO vehicle and 3′-OH fluorescein end-labeled; 2B: CRL1619 melanoma cells treated with 5 μM disulfiram and 3′-OH fluorescein end-labeled).

FIG. 3, shows complexation of copper(II) reduces the antiproliferative activity of disulfiram.

FIG. 4, shows the supplementation of growth medium with copper(II) or zinc(II) enhances the antiproliferative activity of disulfiram.

FIG. 5, shows disulfiram combined with copper(II) induces S-phase cell cycle arrest in CRL1619 melanoma cells and apoptosis.

FIG. 6, shows an X-Ray crystallographic structure of dichlorodiethyldithiocarbamato gold(III).

FIG. 7, shows disulfiram and metals inhibiting transcription factor binding to the cyclic AMP response element. (7A: CRL1619 melanoma cells exhibiting constitutive DNA binding activity to the cyclic AMP response element (CRE) (lane 1); 7B: Treatment of melanoma cells with disulfiram and copper(II) inhibiting transcription factor binding to CRE; 7C: The inhibitory effects of disulfiram or disulfiram plus copper(II) on transcription factor binding are potentiated in the presence of glutathione (GSH).

FIG. 8, shows disulfiram and copper(II) reducing the expression of the cell-cycle protein cyclin A.

FIG. 9, shows disulfiram plus zinc(II) supplementation decreases malignant melanoma growth in mice.

FIG. 10, shows disulfiram and zinc(II) gluconate reducing hepatic tumor volume in a patient with metastatic ocular melanoma.

FIG. 11, shows the X-ray crystallographic structure of [AuCl₂(DEDTC)], which is formed by mixing a diethyldithiocarbamate salt, such as ammonium diethyldithiocarbamate, with tetrachloroauric acid followed by appropriate workup. DEDTC is diethyldithiocarbamate.

FIG. 12, shows the X-ray crystallographic structure of [AuBr₂(DEDTC)], which is formed by mixing diethyldithiocarbamate salt, such as ammonium diethyldithiocarbamate, with tetrabromoauric acid, followed by appropriate workup.

FIG. 13, shows the X-ray crystallographic structure of [Pt(NH₃)(NO₂)(DEDTC)], which is formed by mixing diethyldithiocarbamate salt, such as ammonium diethyldithiocarbamate, with diammineplatinum(II) nitrite, followed by appropriate workup.

FIG. 14, shows the X-ray crystallographic structure of [Fe(DEDTC)₃], which is formed by mixing diethyldithiocarbamate salt, such as ammonium diethyldithiocarbamate, with iron(III) nitrate nonahydrate, followed by appropriate workup.

FIG. 15, shows the X-ray crystallographic structure of [Ga(DEDTC)₃], which is formed by mixing diethyldithiocarbamate salt, such as ammonium diethyldithiocarbamate, with gallium(III) nitrate, followed by appropriate workup.

FIG. 16, shows the X-ray crystallographic structure of [Mn(DEDTC)₃], which is formed by mixing diethyldithiocarbamate salt, such as ammonium diethyldithiocarbamate, with manganese(II) chloride, followed by appropriate workup.

FIG. 17, shows the X-ray crystallographic structure of [Cu(DEDTC)₂], which is formed by mixing diethyldithiocarbamate salt, such as ammonium diethyldithiocarbamate, with copper(II) chloride, followed by appropriate workup.

FIG. 18, shows the X-ray crystallographic structure of [Pt(DEDTC)₂], which is formed by mixing diethyldithiocarbamate salt, such as ammonium diethyldithiocarbamate, with diammineplatinum(II) nitrite followed by appropriate workup.

### DETAILED DESCRIPTION OF THE INVENTION

As described above, the invention provides a neutral compound of formula (I)
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This invention may, however, be embodied in many different forms and should not be construed as limited to the embodiments set forth herein; rather, these embodiments are provided so that this disclosure will be thorough and complete, and will fully convey the scope of the invention to those skilled in the art.

In one aspect, R¹ and R² at each occurrence are independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocyclyl, heterocycloalkyl, aryl, or heteroaryl. In another embodiment, R¹ and R² at each occurrence are independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl.

In yet another aspect, R¹ and R² at each occurrence are independently selected from the group consisting of C₁-C₆ alkyl, C₁-C₆ alkoxy, C₂-C₆ alkenyl with one to three double bonds, C₂-C₆ alkynyl with one or two triple bonds, C₃-C₈ cycloalkyl, aryl, heteroaryl, heterocyclyl, heterocycloalkyl and heterocyclyl.

Preferably, the C₁-C₆ alkyl group is methyl, ethyl, propyl, isopropyl, n-butyl, sec-butyl, tert-butyl, pentyl, 2-pentyl, isopentyl, neopentyl, hexyl, 2-hexyl, 3-hexyl or 3-methylpentyl. Also preferably, the C₁-C₆ alkoxy group is methoxy, ethoxy, propoxy, isopropoxy, n-butoxy, sec-butoxy, tert-butoxy, pentoxy, isopentoxy, neopentoxy, hexoxy or 3-methylpentoxy. Further, the C₂-C₆ alkenyl group is preferably ethenyl, propenyl, 1-but-3-enyl, 1-pent-3-enyl or 1-hex-5-enyl. The C₂-C₆ alkynyl group is preferably ethynyl, propynyl, butynyl or pentyn-2-yl. The cycloalkyl group is preferably cyclopropyl, cyclobutyl, cyclopentyl or cyclohexyl. The aryl group is preferably phenyl, 1-naphthyl, 2-naphthyl, indanyl, indenyl, dihydronaphthyl, tetralinyl or 6,7,8,9-tetrahydro-5H-benzo[α]cycloheptenyl.

In yet another aspect, the heteroaryl group is preferably pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide or benzothiopyranyl S,S-dioxide.

In another embodiment, the heterocyclyl or heterocycloalkyl group is preferably a carbocyclic ring system of 4-, 5-, 6-, or 7-membered rings, which includes fused ring systems of 9-11 atoms containing at least one and up to four heteroatoms selected from nitrogen, oxygen, or sulfur. More preferably, the heterocycloalkyl or heterocyclyl group is morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide.

In a most preferred aspect, R¹ and R² are ethyl.

In another embodiment, M is a main group metal, a transition metal, a lanthanide, or an actinide. More preferably, M is selected from the group consisting of arsenic, bismuth, gallium, manganese, selenium, zinc, titanium, vanadium, chromium, iron, cobalt, nickel, copper, silver, platinum and gold. In a further preferred embodiment, M is gold(III) or copper(II). In another preferred embodiment, M is copper(II). In yet another preferred embodiment, M is platinum(II).

In general, the invention includes compounds wherein A (or multiple A's) is a suitable anionic ligand. More particularly, the invention encompasses compounds wherein A is an anionic ligand selected from the group consisting of Cl⁻, Br⁻, F⁻, I⁻, NO₂⁻, ⁻OR³, ⁻SR³, ⁻N(R³)₂ or ⁻P(R³)₂, or a mixture thereof, wherein R³ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R³ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. In yet another preferred embodiment, R³ is independently hydrogen, methyl, ethyl, isopropyl, tert-butyl, or phenyl. In another aspect, A is an organic-based anionic ligand, such as acetate, formate, oxalate, tartrate, lactate, and the like, or a mixture thereof. In a preferred aspect, A is an anionic ligand selected from the group consisting of Cl⁻, Br⁻, F⁻ and I⁻, or a mixture thereof.

In general, the invention includes compounds where B is any suitable neutral ligand. More particularly, the invention further encompasses compounds wherein the B ligand is a neutral ligand independently selected from the group consisting of NH₃, (R⁴)₂O, N(R⁴)₃, p(R⁴)₃ and (R⁴)₂S, or a mixture thereof, wherein R⁴ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R⁴ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. In yet another preferred embodiment, R⁴ is independently H, methyl, ethyl, isopropyl, tert-butyl, or phenyl.

Further the invention includes compounds wherein C is any suitable cationic ligand, such as for example NO⁺ and NO₂⁺.

The invention also includes compounds wherein each independent (S₂CNR¹R²) portion of the compound of formula (I) is bound to the metal ion through one or both sulfur atoms.

The invention includes compounds wherein M is a metal ion with a coordination number of two and is generally represented by the formulae: 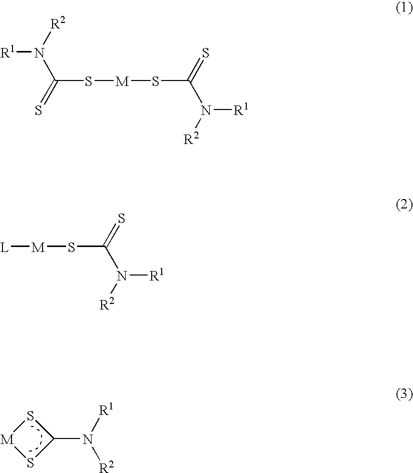
wherein L is a ligand selected from A, B or C, where such ligands are as defined above or below, and R¹ and R² at each occurrence are independently as defined above or below.

In another embodiment, the invention includes compounds wherein M is a metal ion with a coordination number of three and is generally represented by the formulae: 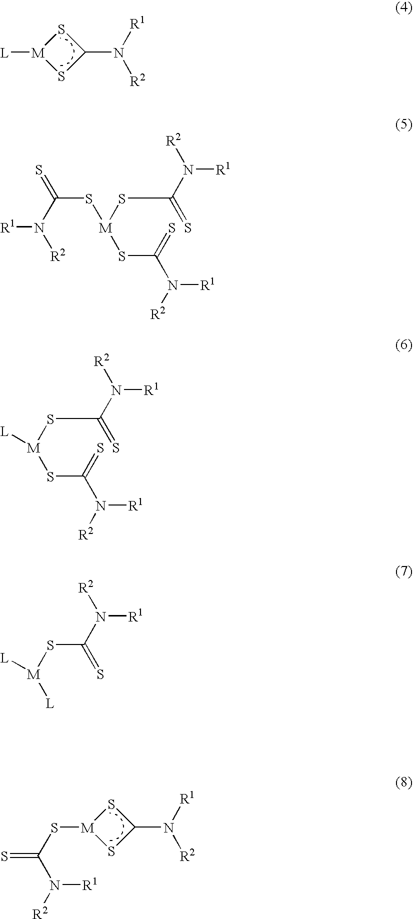
wherein L is a ligand selected from A, B or C, where such ligands are as defined above or below, and R¹ and R² at each occurrence are independently as defined above or below.

In yet another aspect, the invention includes compounds wherein M is a metal ion with a coordination number of four and is generally represented by the formulae: 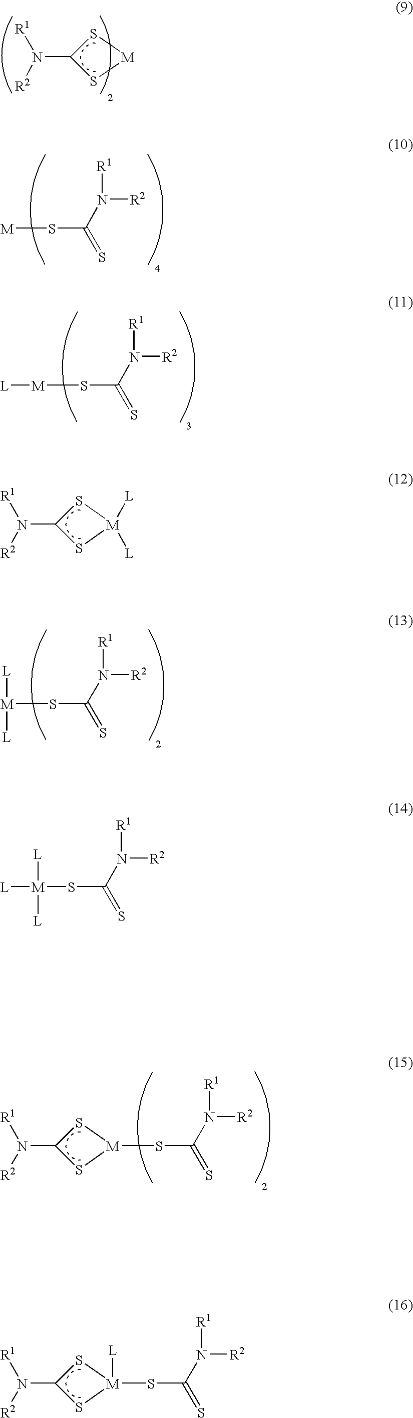
wherein L is a ligand selected from A, B or C, where such ligands are as defined above or below, and R¹ and R² at each occurrence are independently as defined above or below.

The invention also provides compounds, wherein M is a metal ion with a coordination number of five and is generally represented by the formulae: 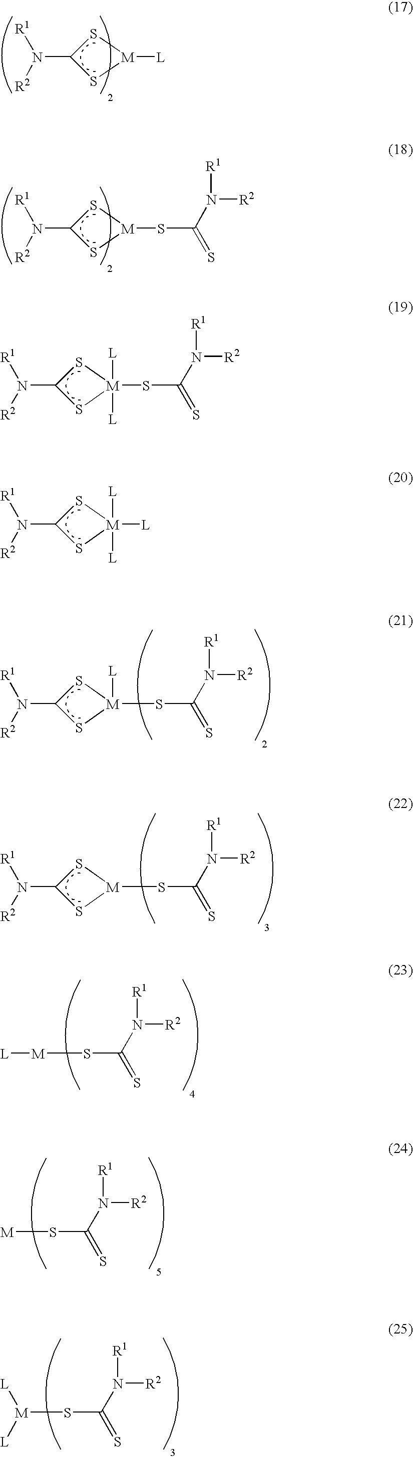 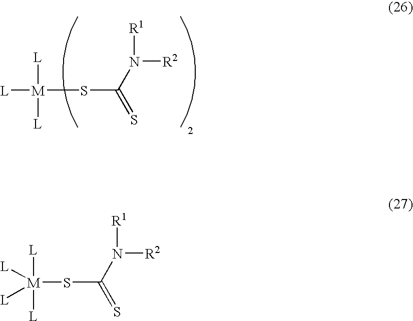
wherein L is a ligand selected from A, B or C, where such ligands are as defined above or below, and R¹ and R² at each occurrence are independently as defined above or below.

Other neutral compounds include those where M is a metal ion with a coordination number of six and is generally represented by the formulae: 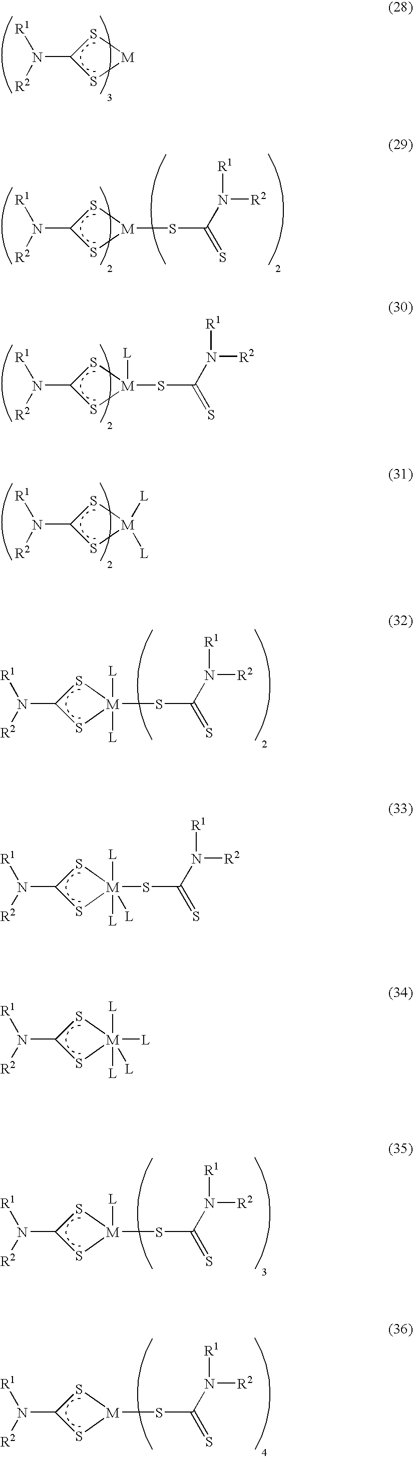 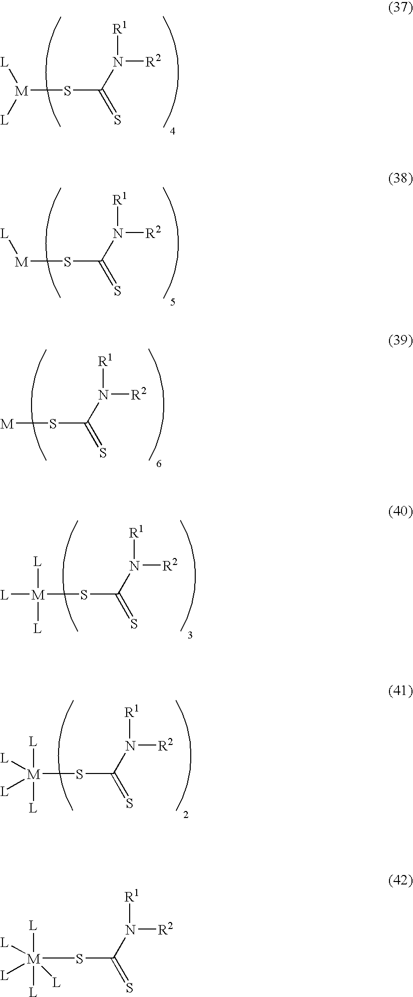
.

As will be appreciated by those of ordinary skill in the art, higher coordination number compounds, i.e., those wherein M is metal ion with a coordination number of 7, 8, 9 and 10 are also encompassed by the invention.

The invention includes a compound of the formula (III):

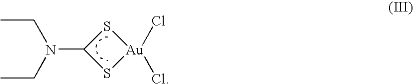

The invention also includes a compound of the formula (IV):

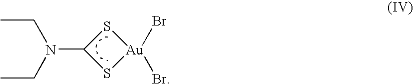

The invention further includes a compound of the formula (V):

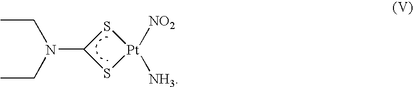

The invention also includes a compound of the formula (Va):

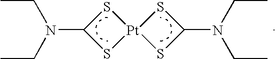

In another aspect, the invention provides compounds of the formula (VI): 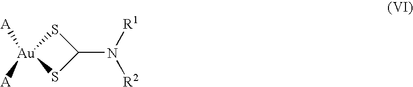
wherein each A, R¹ and R² is independently as defined above or below. In a more preferred aspect, each A is independently a ligand selected from the group consisting of Cl⁻, Br⁻, F⁻, I⁻ and NO₂⁻.

In another embodiment, the invention provides compounds of the formula (VII): 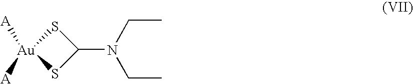
wherein each A is independently as defined above or below. More preferred aspect, each A is independently a ligand selected from the group consisting of Cl⁻, Br⁻, F⁻, I⁻ and NO₂⁻.

Ideally, each independent (S₂CNR¹R²) portion of the compound is of the formula (VIII): 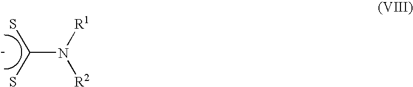
and is bound to M through one or both sulfur atoms. Preferably, the binding is through both sulfur atoms.

The invention provides a pharmaceutical formulation comprising at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge; and
wherein each (S₂CNR¹R²) portion of the compound is bound to the metal ion through one or both sulfur atoms; and
a pharmaceutically acceptable excipient, diluent, solubilizer, solvent, adjuvant, carrier or a mixture thereof. Each R¹ and R² may be the same or different; each A, B and C may be the same or different.

In another embodiment, the invention provides pharmaceutical compositions comprising a compound of the formula (I), wherein R¹ and R² at each occurrence are independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R¹ and R² at each occurrence are independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. More preferably, R¹ and R² are independently selected from the group consisting of C₁-C₆ alkyl, C₁-C₆ alkoxy, C₂-C₆ alkenyl with one to three double bonds, C₂-C₆ alkynyl with one or two triple bonds, C₃-C₈ cycloalkyl, aryl, heteroaryl, heterocycloalkyl and heterocyclyl. Preferably, the C₁-C₆ alkyl group is methyl, ethyl, propyl, isopropyl, n-butyl, sec-butyl, tert-butyl, pentyl, 2-pentyl, isopentyl, neopentyl, hexyl, 2-hexyl, 3-hexyl or 3-methylpentyl. The C₁-C₆ alkoxy group is preferably methoxy, ethoxy, propoxy, isopropoxy, n-butoxy, sec-butoxy, tert-butoxy, pentoxy, isopentoxy, neopentoxy, hexoxy or 3-methylpentoxy. The C₂-C₆ alkenyl group is preferably ethenyl, propenyl, 1-but-3-enyl, 1-pent-3-enyl or 1-hex-5-enyl. The C₂-C₆ alkynyl group is preferably ethynyl, propynyl, butynyl or pentyn-2-yl. The cycloalkyl group is cyclopropyl, cyclobutyl, cyclopentyl or cyclohexyl.

Pharmaceutical compositions comprising a neutral compound of the formula (I), include those where the aryl group is phenyl, 1-naphthyl, 2-naphthyl, indanyl, indenyl, dihydronaphthyl, tetralinyl or 6,7,8,9-tetrahydro-5H-benzo[α]cycloheptenyl. Preferably, the heteroaryl group is pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide or benzothiopyranyl S,S-dioxide.

Pharmaceutical compositions comprising a neutral compound of the formula (I) also include those wherein the heterocycloalkyl or heterocyclyl is a carbocyclic ring system of 4-, 5-, 6-, or 7-membered rings which includes fused ring systems of 9-11 atoms containing at least one and up to four heteroatoms selected from nitrogen, oxygen, or sulfur. Preferably, the heterocycloalkyl or heterocyclyl group is morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide.

Most preferred pharmaceutical formulations are those in which for compounds of formula (I), R¹ and R² are ethyl. Other preferred pharmaceutical formulations include those wherein M is a main group metal, a transition metal, a lanthanide or an actinide. More preferably, M is selected from the group consisting of arsenic, bismuth, gallium, manganese, selenium, zinc, titanium, vanadium, chromium, iron, cobalt, nickel, copper, silver, platinum(II) and gold. In a further preferred embodiment, M is gold(III) or copper(II). In another preferred embodiment, M is copper(II). In yet another preferred embodiment, M is platinum(II).

The invention encompasses pharmaceutical formulations comprising compounds of formula (I), wherein A is a suitable anionic ligand. More particularly, the invention encompasses compounds wherein A is an anionic ligand selected from the group consisting of Cl⁻, Br⁻, F⁻, I⁻, NO₂⁻, ⁻OR³, ⁻SR³, ⁻N(R³)₂ or ⁻P(R³)₂, or a mixture thereof, wherein R³ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R³ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. In yet another preferred embodiment, R³ is independently H, methyl, ethyl, isopropyl, tert-butyl, or phenyl. In another aspect, A is an organic-based anionic ligand, such as acetate, formate, oxalate, tartrate, lactate, and the like, or a mixture thereof. In a preferred aspect, A is an anionic ligand selected from the group consisting of Cl⁻, Br⁻, F⁻ and I⁻, or a mixture thereof.

More particularly, the invention further encompasses pharmaceutical formulations utilizing compounds wherein the B ligand is a neutral ligand independently selected from the group consisting of NH₃, (R⁴)₂O, N(R⁴)₃, P(R⁴)₃ and (R⁴)₂S, or a mixture thereof, wherein R⁴ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R⁴ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. In yet another preferred embodiment, R⁴ is independently H, methyl, ethyl, isopropyl, tert-butyl, or phenyl.

Further the invention includes pharmaceutical formulation utilizing compounds wherein C is any suitable cationic ligand, such as for example NO⁺ and NO₂⁺.

The invention also includes pharmaceutical formulations comprising compounds of formula (I), wherein each independent (S₂CNR¹R²) portion of the compound of formula (I) is bound to the metal ion through one or both sulfur atoms.

The invention includes a pharmaceutical formulation comprising a compound of formulae 1-42 above, i.e., a compound wherein M is a metal ion with a coordination number of 2-6; wherein L is a ligand selected from A, B or C, where such ligands are as defined above or below, and R¹ and R² at each occurrence are independently as defined above or below. Pharmaceutical formulations comprising compounds with higher coordination numbers, i.e., those where M is metal ion with a coordination number of 7, 8, 9 and 10 are also encompassed by the invention.

The invention includes a pharmaceutical formulation comprising a compound of the formula (III).

The invention also includes a pharmaceutical formulation comprising a compound of the formula (IV).

The invention further includes a pharmaceutical formulation comprising a compound of the formula (V) or (Va).

In another aspect, the invention provides a pharmaceutical formulation comprising a compound of the formula (VI), wherein each A, R¹ and R² are independently as defined above or below. In a preferred aspect, each A is independently a ligand selected from the group consisting of Cl³¹ , Br⁻, F⁻, I⁻ and NO₂⁻.

In another embodiment, the invention provides a pharmaceutical formulation comprising a compound of the formula (VII), wherein each A is as defined above or below. In a preferred aspect, each A is independently a ligand selected from the group consisting of Cl⁻, Br⁻, F⁻, I⁻ and NO₂⁻.

Ideally, in the pharmaceutical formulation comprising a compound of the formula (I), each independent (S₂CNR¹R²) portion of the compound is of the formula (VIII) and is bound to M through one or both sulfur atoms.

The pharmaceutical formulations can be in the form of tablets, pills, powders, elixirs, suspensions, emulsions, solutions, syrups, capsules (such as, for example, soft and hard gelatin capsules), suppositories, sterile injectable solutions, and sterile packaged powders.

The invention encompasses a method of treating cancer in an animal comprising administering to an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method is preferably suited to treatment of cancers selected from but not limited to the group of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. These methods are most preferably suited to treatment of cancers selected from the group of melanoma, lung cancer, breast cancer, colon and prostate cancer.

This method may also include the various embodiments and preferred embodiments as described above or below.

In a preferred aspect of this method, the animal is a mammal; in a more preferred aspect the mammal is a human. Further, the therapeutically effective amount is administered in a dosage of between about 1 mg to about 1000 mg per day, based upon body weight. More preferably, the therapeutically effective amount comprises a dosage of between about 25 mg to about 500 mg per day, based upon body weight.

In another aspect of this method, the therapeutically effective amount of the compound is administered parenterally. Alternatively, the therapeutically effective amount of the compound is administered orally.

In another embodiment of this method, R¹ and R² are ethyl. Preferably, M is a main group metal, a transition metal, a lanthanide or an actinide. More preferably, M is selected from the group consisting of arsenic, bismuth, gallium, manganese, selenium, zinc, titanium, vanadium, chromium, iron, cobalt, nickel, copper, silver, platinum(II) and gold. In a further preferred embodiment, M is gold(III) or copper(II). In another preferred embodiment, M is copper(II). In yet another preferred embodiment, M is platinum(II).

This method embodiment further utilizes compounds of formula (I), wherein A is an anionic ligand selected from the group consisting of Cl⁻, Br⁻, F⁻, I⁻, NO₂⁻, ⁻OR³, ⁻SR³, ⁻N(R³)₂ or ⁻P(R³)₂, or a mixture thereof, wherein R³ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R³ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. In yet another preferred embodiment, R³ is independently H, methyl, ethyl, isopropyl, tert-butyl, or phenyl. In another aspect, A is an organic-based anionic ligand, such as acetate, formate, oxalate, tartrate, lactate, and the like, or a mixture thereof. In a preferred aspect, A is an anionic ligand selected from the group consisting of Cl⁻, Br⁻, F⁻ and I⁻, or a mixture thereof.

This method further encompasses the use of compounds of formula (I), wherein B is a neutral ligand independently selected from the group consisting of NH₃, (R⁴)₂O, N(R⁴)₃, P(R⁴)₃ and (R⁴)₂S, or a mixture thereof, wherein R⁴ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl. In another embodiment, R⁴ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl. In yet another preferred embodiment, R⁴ is independently H, methyl, ethyl, isopropyl, tert-butyl, or phenyl.

Further, this method encompasses use of compounds of formula (I), wherein C is a cationic ligand, such as for example NO⁺ and NO₂⁺.

This method also include use of compounds of formula (I), wherein each independent (S₂CNR¹R²) portion of the compound of formula (I) is bound to the metal ion through one or both sulfur atoms.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10 are also encompassed by the invention.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br³¹ , F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

In accordance with this method, the invention includes a method wherein the cancer is a multidrug-resistant.

The invention encompasses a method of treating cancer in animals comprising administering to an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable excipient, diluent, solubilizer, solvent, adjuvant or carrier, or a mixture thereof. Each R¹ and R² may be the same or different; each A, B and C may be the same or different.

This method are preferably suited to treatment of cancers selected from but not limited to the group of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. The method is most preferably suited to treatment of cancers selected from the group of melanoma, lung cancer, breast cancer, colon and prostate cancer.

This method may also include the various embodiments and preferred embodiments as described above or below.

In a preferred aspect of this method, the animal is a mammal. In a more preferred aspect, the mammal is a human. Further, the therapeutically effective amount is administered in a dosage as described above or below; the therapeutically effective amount of the pharmaceutical formulation is administered as described above or below.

This method also include use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

In accordance with this method, the invention includes a method wherein the cancer is a multidrug-resistant.

The invention includes a method for treating cancer in an animal, and for treating, removing or preventing multi-drug resistance in the animal, comprising administering to an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method is preferably suited to treatment of cancers selected from but not limited to the group of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. The method is most preferably suited to treatment of cancers selected from the group of melanoma, lung cancer, breast cancer, colon and prostate cancer.

In accordance with this method and the methods below, sensitization means directly promoting cancer cell death as mediated by the metal ion complex. In accordance with this method and the methods below, potentiating means where the metal ion complex works in concert with other chemotherapeutic or non-chemotherapeutic compounds to promote cancer cell death.

This method may also include the various embodiments and preferred embodiments as described above or below.

In a preferred aspect of this method, the animal is a mammal; more preferably, the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of C⁻, Br⁻, F⁻, I³¹ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention also includes a method for treating cancer in an animal, and for treating, removing or preventing multi-drug resistance in the animal, comprising administering to the animal in need of such treatment, a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R ²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method is preferably suited to treatment of cancers selected from but not limited to the group of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. The method is most preferably suited to treatment of cancers selected from the group of melanoma, lung cancer, breast cancer, colon and prostate cancer.

In accordance with this method and the methods below, sensitization means directly promoting cancer cell death as mediated by the metal ion complex. In accordance with this method and the methods below, potentiating means where the metal ion complex works in concert with other chemotherapeutic or non-chemotherapeutic compounds to promote cancer cell death.

This method may also include the various embodiments and preferred embodiments as described above or below.

In a preferred aspect of this method, the animal is a mammal; more preferably, the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method of sensitizing and/or potentiating cancerous tumors to conventional cancer chemotherapy or radiation therapy comprising administering to an animal with such tumors and in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method is preferably suited to treatment of cancers selected from but not limited to the group of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. The method is most preferably suited to treatment of cancers selected from the group of melanoma, lung cancer, breast cancer, colon and prostate cancer.

In accordance with this method and the methods below, sensitization means directly promoting cancer cell death as mediated by the metal ion complex. In accordance with this method and the methods below, potentiating means where the metal ion complex works in concert with other chemotherapeutic or non-chemotherapeutic compounds to promote cancer cell death.

This method may also include the various embodiments and preferred embodiments as described above or below.

In a preferred aspect of this method, the animal is a mammal; more preferably, the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method of sensitizing and/or potentiating cancerous tumors to conventional cancer chemotherapy or radiation therapy comprising administering to an animal with such tumors and in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method is preferably suited to treatment of cancers selected from but not limited to the group of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases. The method is most preferably suited to treatment of cancers selected from the group of melanoma, lung cancer, breast cancer, colon and prostate cancer.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, the method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula VIII and is bound to M through one or both sulfur atoms.

The invention encompasses a method for sensitizing patients with compromised immune systems, such as for example, patients with HIV, AIDS, to anti-retroviral therapy comprising administering to a human in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R may be the same or different; each A, B and C may be the same or different.

This method may also include the various embodiments and preferred embodiments as described above or below.

The therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method for sensitizing patients with compromised immune systems, such as for example, patients with HIV, AIDS, to anti-retroviral therapy comprising administering to a human in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

The therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method of reducing hypoxic or ischemic damage to the cardiovascular system of an animal comprising administering to an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

As used in accordance with this method and the methods below, reducing hypoxic or ischemic damage means to bring down, as in extent, amount or degree or to diminish, such damage. See, The American Heritage Dictionary, 3^{rd} Ed., 1994.

As used in accordance with this method and the methods below, hypoxic or ischemic damage includes, but is not limited to, conditions arising due to a decrease below normal levels of oxygen in inspired gases, arterial blood, or tissue, short of anoxia for hypoxia, conditions or processes leading to mechanical obstruction (mainly arterial narrowing) of the blood supply for ischemia, diseases of ischemia-reperfusion injury (such as stroke, myocardial infarction, organ injury incurred during preservation before transplantation), acute renal failure, hemorrhagic shock with total body reperfusion after fluid resuscitation to restore normal blood pressure and tissue perfusion.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula VII, wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method of reducing hypoxic or ischemic damage to the cardiovascular system of an animal comprising administering to an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R 2) portion of the compound is of the formula (VIII) and is bound to M through one or both sulfur atoms.

The invention encompasses a method for treating asthma in animals comprising administering to an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

As used herein, the term asthma means inflammation of the airway resulting in reversible or irreversible obstruction of the airway luminal size and/or pulmonary disease states, which include but are not limited to disease states which are based upon micro-cilliary transport defects, and other conditions leading to a difficulty in breathing in the affected individual.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method of treating asthma in animals comprising administering to an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method for treating arthritis, such as for example rheumatoid arthritis, osteoarthritis, and arthritis from other connective tissue diseases, including Sjorgren's syndrome, systemic lupus erythematosis, polymyositis, dermatomyositis, mixed connective tissue disease and overlap syndromes, comprising administering to an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention encompasses a method of treating arthritis, such as for example rheumatoid arthritis, osteoarthritis, and arthritis from other connective tissue diseases, including Sjorgren's syndrome, systemic lupus erythematosis, polymyositis, dermatomyositis, mixed connective tissue disease and overlap syndromes, comprising administering to an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention also encompasses a method of treating proliferative dermatologic conditions, utilizing compounds of formula (I), such as for example, topical treatment of actinic keratosis, squamous or basal cell cancer and psoriasis. This method comprises administering to or applying on an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention also encompasses a method of treating proliferative dermatologic conditions utilizing pharmaceutical formulations comprising a neutral compound of formula (I) such as for example, topical treatment of actinic keratosis, squamous or basal cell cancer and psoriasis. This method comprises administering to or applying on an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention further includes a method of treating conditions and ailments which may relate in part to inhibition of NADPH oxidases, utilizing compounds of formula (I), such as for example, hypertension, diabetic vascular disease, angiogenesis (including tumor angiogenesis), atherosclerosis, proliferative diabetic retinopathy, macular degeneration (especially the “wet” variety), vascular restenosis following angioplasty/stenting (wherein the metal complex, such as a copper complex is doped upon a stent to produce a drug-eluting stent) and ischemia-reperfusion injury syndrome (such as myocardial infarction, stroke, acute renal failure and the like). This method comprises administering to or applying on an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention further encompasses a method of treating conditions and ailments which may relate in part to inhibition of NADPH oxidases, utilizing pharmaceutical formulations comprising a neutral compound of formula (I), such as for example, hypertension, diabetic vascular disease, angiogenesis (including tumor angiogenesis), atherosclerosis, proliferative diabetic retinopathy, macular degeneration (especially the “wet” variety), vascular restenosis following angioplasty/stenting (wherein the metal complex, such as a copper complex is doped upon a stent to produce a drug-eluting stent) and ischemia-reperfusion injury syndrome (such as myocardial infarction, stroke, acute renal failure and the like). This method comprises administering to or applying on an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention further includes a method of treating diseases of inflammation, where NF-kappa B, AP-1 and ATF/CREB are activated and play roles in mediating inflammatory processes, utilizing compounds of formula (I), such as for example, asthma, arthritis (including rheumatoid disease, systemic lupus, mixed connective tissue disease, overlap syndromes, and the like), sarcoidosis, chronic active hepatitis, glomerulonephritis, eczema, poison ivy, chronic interstitial lung disease, inflammatory bowel diseases (ulcerative colitis and Crohn's disease) and acute lung injury and adult respiratory distress syndrome. This method comprises administering to or applying on an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention further includes a method of treating diseases of inflammation, where NF-kappa B, AP-1 and ATF/CREB are activated and play roles in mediating inflammatory processes, utilizing pharmaceutical formulations comprising a neutral compound of formula (I), such as for example, asthma, arthritis (including rheumatoid disease, systemic lupus, mixed connective tissue disease, overlap syndromes, and the like), sarcoidosis, chronic active hepatitis, glomerulonephritis, eczema, poison ivy, chronic interstitial lung disease, inflammatory bowel diseases (ulcerative colitis and Crohn's disease) and acute lung injury and adult respiratory distress syndrome. This method comprises administering to or applying on an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention further includes a method of treating degenerative diseases related to activation of caspases, utilizing compounds of formula (I), such as for example, emphysema, Alzheimer's disease, Parkinson's disease and amyotrophic lateral sclerosis. This method comprises administering to or applying on an animal in need of such treatment a therapeutically effective amount of at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a compound of formulae 1-42 above, i.e., compounds wherein M is a metal ion with a coordination number of 2-6; wherein L is a ligand selected from A, B or C, where such ligands are as defined above or below, and R¹ and R² at each occurrence are independently as defined above or below. This method also encompasses use of compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F³¹ , I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention further includes a method of treating degenerative diseases related to activation of caspases, utilizing pharmaceutical formulations comprising a neutral compound of formula (I), such as for example, emphysema, Alzheimer's disease, Parkinson's disease and amyotrophic lateral sclerosis. This method comprises administering to or applying on an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein R¹, R², M, A, B, C, n, x, y and z are as defined above or below, where when n is greater than 1, each (S₂CNR¹R²) may be the same or different; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; and wherein each (S₂CNR¹R²) portion of the compound is bound to the metal ion through one or both sulfur atoms; and a pharmaceutically acceptable carrier, excipient, solvent, adjuvant or diluent. Each R¹ and R² may be the same or different; each A, B and C may be the same or different.

This method may also include the various embodiments and preferred embodiments as described above or below.

In a preferred aspect of this method, the animal is a mammal; more preferably the mammal is a human. Further, the therapeutically effective amount is administered in a dosage as described above or below; the therapeutically effective amount of the pharmaceutical formulation is administered as described above or below.

This method also includes use of a pharmaceutical formulation comprising a compound of formulae 1-42 above, i.e., compounds wherein M is a metal with a coordination number of 2-6; wherein L is a ligand selected from A, B or C, where such ligands are as defined above or below, and R¹ and R² at each occurrence are independently as defined above or below. This method further includes use of pharmaceutical formulations comprising compounds with higher coordination numbers, i.e., those where M is a metal ion with a coordination number of 7, 8, 9 and 10.

This method includes the use of a compound of formula (III).

This method also includes the use of a compound of the formula (IV).

This method also includes the use of a compound of the formula (V) or (Va).

In another aspect, this method utilizes a compound of the formula (VI), wherein each A, R¹ and R² are independently as defined above or below. In a preferred aspect, each A is independently a ligand selected from the group consisting of Cl⁻, Br⁻, F⁻, I⁻ and NO₂⁻.

In another embodiment, this method utilizes a compound of the formula (VII), wherein each A is as defined above or below. In a preferred aspect of this method, each A is independently a ligand selected from the group consisting of Cl⁻, Br⁻, F⁻, I⁻ and NO₂⁻.

Ideally, when the compound of formula (I) is utilized in this method, each independent (S₂CNR¹R²) portion of the compound is of the formula (VIII) and is bound to M through one or both sulfur atoms.

The invention includes the use of a compound of formula (I) or a pharmaceutical formulation comprising a compound of formula (I), as an antifungal agent which can be applied to a mammal, such as a human, either topically or administered systemically.

The invention also includes a method of making a compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different; and
wherein each A, B and C may be the same or different.

Further, the invention encompasses a pharmaceutical composition in unit dosage form comprising at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different; and
wherein each A, B and C may be the same or different.

In accordance with this embodiment, the pharmaceutical composition in unit dosage form comprises a pharmaceutically acceptable excipient, diluent, solubilizer, solvent, adjuvant or carrier, or a mixture thereof. Preferably, the neutral compound of formula (I) is present in an amount of about 1.0 mg to about 1000 mg. more preferably, the neutral compound of formula (I) is present in an amount of about 25 mg to about 500 mg.

Generally, as used herein, the term “dithiocarbamate disulfides” refers to compounds having the formula (IX): 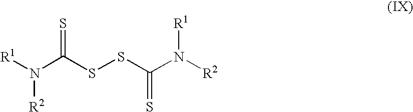
wherein each R¹ and R² is independently as defined above or below. Further, the dithiocarbamate disulfides may be symmetric or asymmetric. In one aspect, each R¹ and R² are independently hydrogen or an organic substituent such as saturated and unsaturated alkyl or aryl groups, or saturated or unsaturated heteroatom containing alkyl or aryl groups; further groups include, for example, unsubstituted or substituted alkyl, alkenyl, alkynyl, aryl, alkoxy, heterocycloalkyl and heteroaryl groups. The two substituents on any or both nitrogens may be incorporated into a saturated or unsaturated heterocyclic ring. Typically R¹ and R² are not both hydrogen. Thus, dithiocarbamate disulfide is a disulfide form of dithiocarbamates that have a reduced sulfhydryl group.

Many dithiocarbamates are known and synthesized in the art. Non-limiting examples of dithiocarbamates include diethyldithiocarbamate (DEDTC), pyrrolodinedithiocarbamate, N-methyl, N-ethyl dithiocarbamates, hexamethylenedithiocarbamate, imidazolinedithiocarbamates, dibenzyldithiocarbamate, dimethylenedithiocarbamate, dipolyldithiocarbamate, dibutyldithiocarbamate, diamyldithiocarbamate, N-methyl, N-cyclopropylmethyldithiocarbamate, cyclohexylamyldithiocarbamate, pentamethylenedithiocarbamate, dihydroxyethyldithiocarbamate, N-methylglucosamine dithiocarbamate, and salts and derivatives thereof. Typically, a sulfhydryl-containing dithiocarbamate can be oxidized to form a dithiocarbamate disulfide.

Sulfhydryl-containing dithiocarbamates can be converted to their corresponding thiolate anions by treatment with an alkali-metal hydroxide as a proton acceptor, yielding the structure: 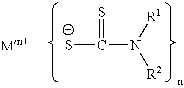
wherein R¹ and R² are defined above, M′ is an alkali metal, alkaline earth metal, or organic or inorganic cation selected from the group consisting of sodium, potassium, calcium, magnesium, barium, lithium, ammonium, mono-, di-, tri- or tetra-alkyl ammonium, or aryl ammonium, tetra-alkyl phosphonium, or aryl phosphonium; and n is the charge on the cation.

Finally, the metal ion coordination compounds of dithiocarbamates can be synthesized either by treatment of the disulfide or the thiolate anion forms of dithiocarbamates with metal ion sources yielding a variety of useful metal compounds in which the dithiocarbamate is a bidentate ligand to the same metal ion: 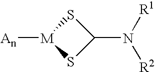
wherein R¹ and R² are defined as above; M is a metal ion, e.g., arsenic, bismuth, gallium, manganese, selenium, zinc, titanium, vanadium, chromium, iron, cobalt, nickel, copper, silver or gold; A is an anionic ligand selected from the group consisting of chloride, bromide, iodide, acetate and low molecular weight organic or inorganic pharmaceutically acceptable anions; n is the number of ligands coordinated to the metal ion. It should be noted that the value of n depends on the coordination number and oxidation state of the metal ion. Typically, n is an integer of 1-10.

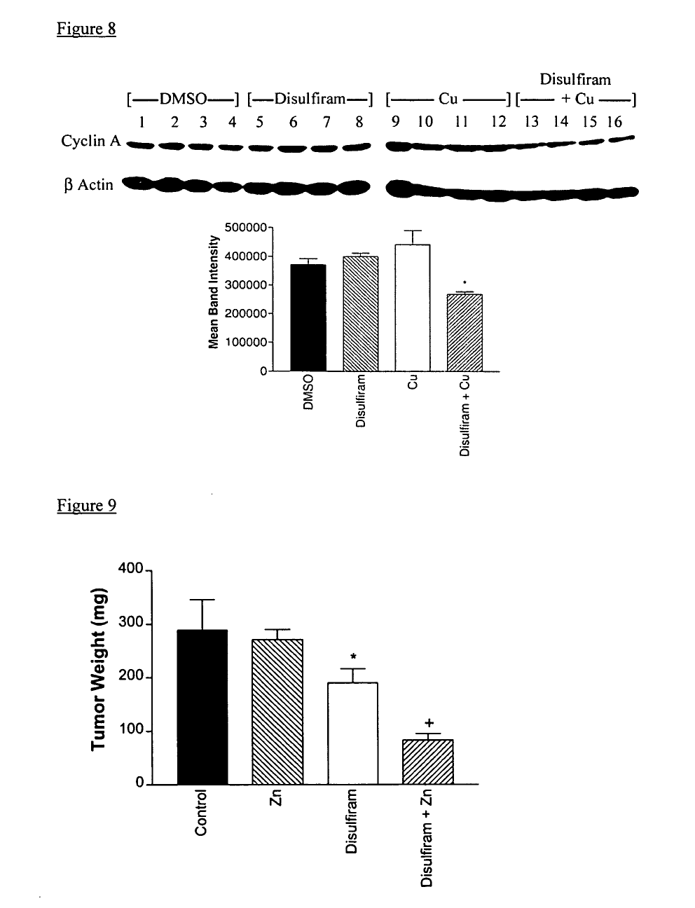

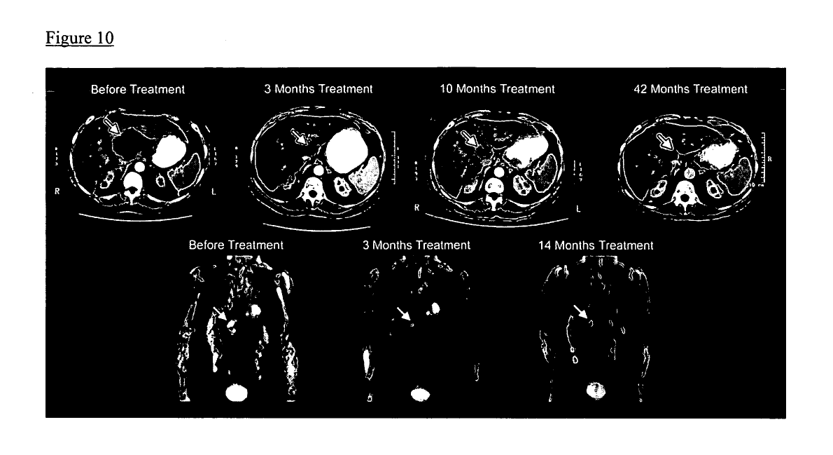

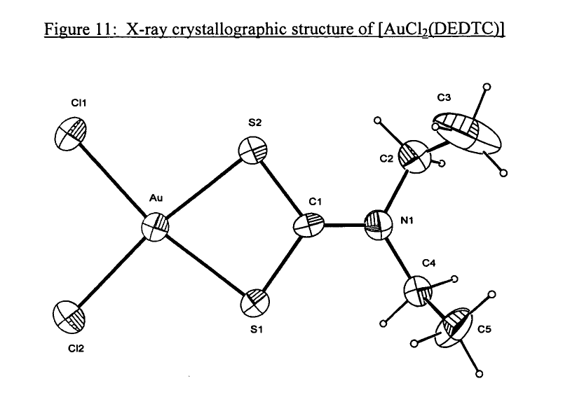

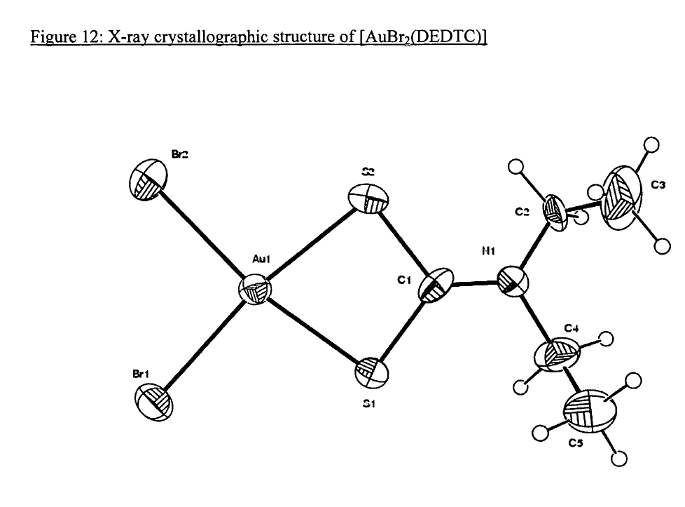

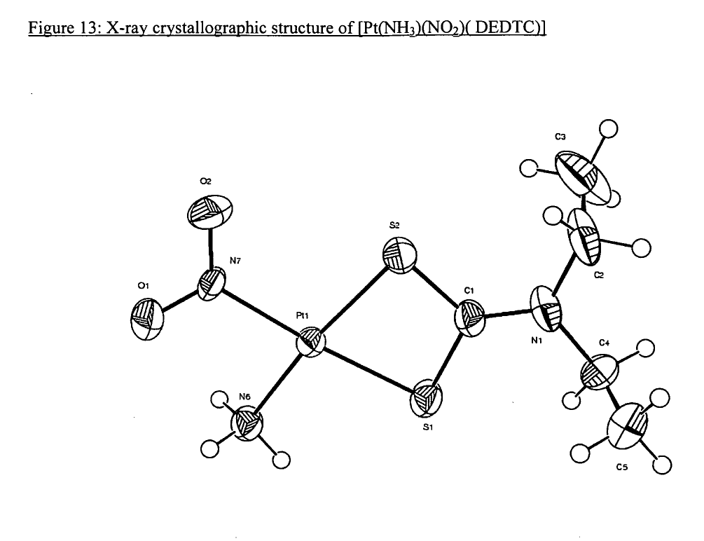

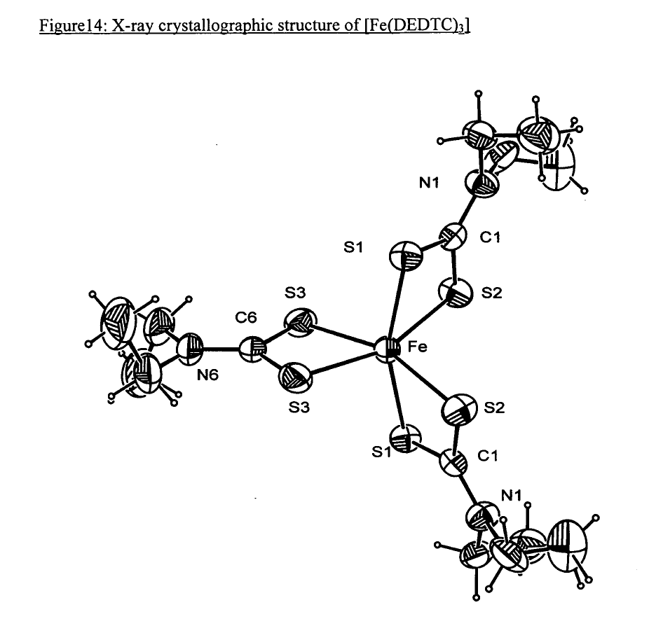

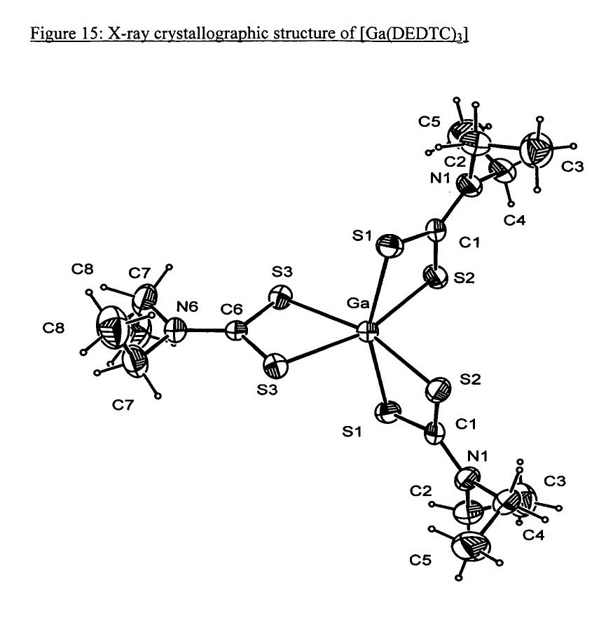

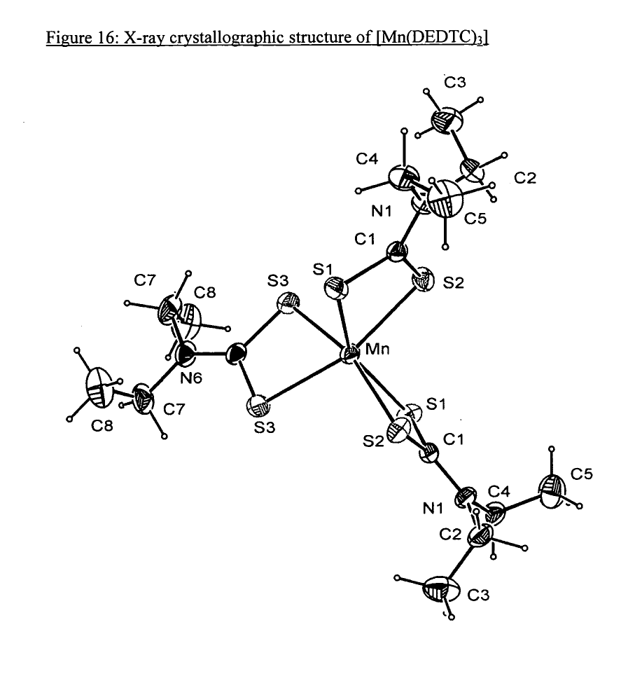

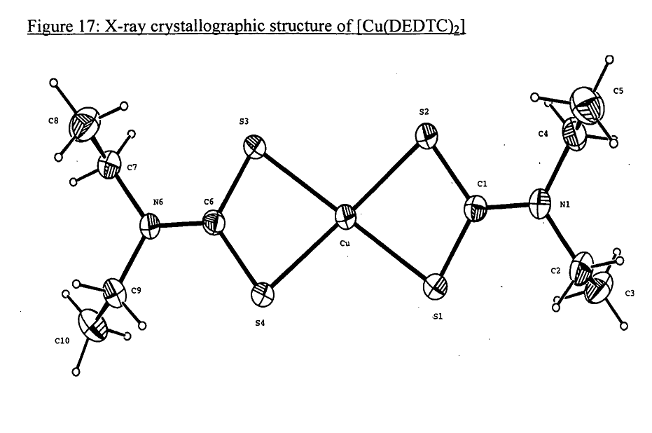

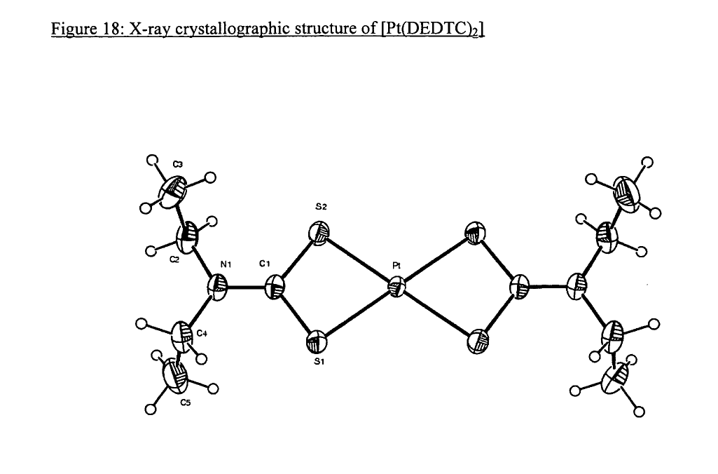

Specifically, the preferred gold(III) dithiocarbamato compounds have the formula: 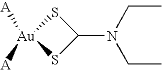
wherein A at each occurrence is independently an anionic ligand of low molecular weight, for example chloride and bromide; see e.g., FIGS. 8 and 9.

Any pharmaceutically acceptable form of dithiocarbamate disulfides, their corresponding thiolate anions, or dithiocarbamate coordination compounds can be used. For example, tetraethylthiuram disulfide, which is known as disulfiram, is used in one embodiment of this invention. Disulfiram has the following formula: 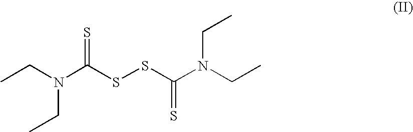
Disulfiram has been used clinically in the treatment of alcohol abuse, in which disulfiram inhibits hepatic aldehyde dehydrogenase.

The thiolate anion derivative of disulfiram is diethyldithiocarbamate anion, the sodium salt of which has the following formula: 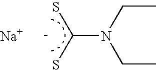
Finally, the compound of diethyldithiocarbamate, exemplified below as the gold(III) 1,1-dithiocarbamato complex, is shown: 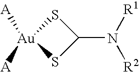
wherein R¹ and R² are ethyl, and wherein A at each occurrence is independently an anionic ligand of low molecular weight. Examples of low molecular weight anionic ligands include, but are not limited to, halide, nitro, amino, hydroxy and the like.

Methods of making dithiocarbamates and their corresponding disulfides are generally known in the art. Exemplary methods are disclosed in, e.g., Thorn, et al, The Dithiocarbamates and Related Compounds, Elsevier, N.Y., 1962; and U.S. Pat. Nos. 5,166,387, 4,144,272, 4,066,697, 1,782,111, and 1,796,977, all of which are incorporated herein by reference.

As used herein, the term “treatment”, or a derivative thereof, contemplates partial or complete inhibition of the stated disease state, when an active ingredient of the invention is administered prophylactically or following the onset of the disease state for which such active ingredient of the is administered. For the purposes of the present invention, “prophylaxis” refers to administration of the active ingredient(s) to a mammal to protect the mammal from any of the disorders set forth herein, as well as others. Further, the term “treating cancer” as used herein, specifically refers to administering a therapeutically appropriate amount of therapeutic agents to a patient diagnosed with cancer, i.e., having established cancer in the patient, to inhibit the further growth or spread of the malignant cells in the cancerous tissue and/or to cause the death of malignant cells. This term also includes prophylactic use, according to the methods of the invention, compounds as described herein, for such cancers including, for example, mammalian breast carcinoma.

This invention provides a method for treating cancer in a patient. In accordance with the present invention, it has been discovered that dithiocarbamate disulfides, their corresponding thiolate anions, and their coordination compounds, such as disulfiram, the diethyldithiocarbamate anion, and dichloro(diethyldithiocarbamato)gold(III), respectively, can inhibit the growth of tumor cells in a metal ion-dependent manner. Specifically, metal ions such as copper(II), zinc(II), gold(III), and silver(I) significantly enhance the inhibitory effect of dithiocarbamate disulfides and their thiolate anions on tumor cells, while depletion of such metal ions prevents growth inhibition by disulfiram and the diethyldithiocarbamate anion. The function performed by the metal ion is to chemically enable formation of or stabilize the thiolate anion form in vivo, so that the thiolate anion is able to form mixed disulfides with protein cysteine sulfhydryl groups of cellular proteins.

In accordance with one aspect of this invention, a method for treating an established cancer in a patient is provided. A dithiocarbamate disulfide can be administered to a patient having established cancer to treat that cancer. Preferably, the thiuram disulfide administered is a tetraalkylthiuram disulfide such as tetraethylthiuram disulfide, i.e., disulfiram.

In another aspect, the method for treating cancer in a patient comprises administering to the patient a therapeutically effective amount of a dithiocarbamate thiolate anion.

Preferably, the dithiocarbamate is administered in the form of a coordination compound. As is known in the art, dithiocarbamates are excellent chelating agents and can bind to metal ions to form chelate compounds. The ordinary artisan knows the synthetic routes towards the coordination compounds of dithiocarbamates. (e.g., D. Coucouvanis, “The chemistry of the dithioacid and 1,1-dithiolate complexes,” Prog. Inorganic Chem. 11:234-371 (1970); D. Coucouvanis, “The chemistry of the dithioacid and 1,1-dithiolate complexes, 1968-1977,” Prog. Inorganic Chem. 26:302-469 (1978); R. P. Burns, et al., “1,1-dithiolato complexes of the transition metals,” Adv. Inorganic Chem. and Radiochem. 23:211-280(1980); L. I. Victoriano, et al., “The reaction of copper (II) chloride and tetralkylthiuram disulfides,” J. Coord. Chem. 35:27-34 (1995); L. I. Victoriano, et al., “Cuprous dithiocarbamates. Syntheses and reactivity,” J. Coord. Chem. 39:231-239 (1996).) For example, dithiocarbamate coordination compounds of copper(II), gallium (III), bismuth (III) and gold(III) ions can be conveniently synthesized by mixing, in suitable solvents, disulfiram or sodium diethyldithiocarbamate or alkyl ammonium diethyldithiocarbamate with, e.g., CuSO₄, CuCl₂, Bi(NO₃)₃, Ga(NO₃)₃, HAuCl₄ or HAuBr₄. Other dithiocarbamate chelate compounds are disclosed in, e.g., D. Coucouvanis, “The chemistry of the dithioacid and 1,1-dithiolate complexes,” Prog. Inorganic Chem. 11:234-371 (1970); D. Coucouvanis, “The chemistry of the dithioacid and 1,1-dithiolate complexes, 1968-1977,” Prog. Inorganic Chem. 26:302-469 (1978); R. P. Burns, et al., “1,1-dithiolato complexes of the transition metals,” Adv. Inorganic Chem. and Radiochem. 23:211-280(1980); L. I. Victoriano, et al., “The reaction of copper (II) chloride and tetralkythiuram disulfides,” J. Coord. Chem. 35:27-34 (1995); L. I. Victoriano, et al., “Cuprous dithiocarbamates. Syntheses and reactivity,” J. Coord. Chem. 39:231-239 (1996), which are incorporated herein by reference.

In accordance with another aspect of this invention, a method for treating cancer in a patient is provided which includes administering to the patient a therapeutically effect amount of a dithiocarbamate anion compound and an intracellular metal ion stimulant, which can enhance the intracellular level of the above described metal ions in the patient. Intracellular heavy metal ion carriers are known. For example, ceruloplasmin can be administered to the patient to enhance the intracellular copper level. Other metal ion carriers known in the art may also be administered in accordance with this aspect of the invention. The heavy metal ion carriers and the dithiocarbamate disulfide or thiolate anion can be administered together or separately, and, preferably, in separate compositions.

Ceruloplasmin is a protein naturally produced by the human body and can be purified from human serum. This 132-kD glycoprotein, which carries 7 copper(II) ions complexed over three 43-45 kD domains, is an acute phase reactant and the major copper-carrying protein in human plasma. See Halliwell, et al., Methods Enzymol. 186:1-85 (1990). When transported into cells, at least some of the bound copper(II) ions can be accessible for complexation with the dithiocarbamate disulfide or thiolate anion administered to the patient. (See Percival, et al., Am. J. Physiol. 258:3140-3146 (1990).) Ceruloplasmin and dithiocarbamate disulfides or thiolate anions are typically administered in different compositions. Dithiocarbamate disulfides or thiolate anions can be administered at about the same time, or at some time apart. For example, ceruloplasmin can be administered from about five minutes to about 12 hours before or after dithiocarbamate disulfide or thiolate anions are administered to the patient.

In another embodiment of this aspect of the invention, instead of heavy metal ion carriers, a cytokine is administered to the patient in addition to a dithiocarbamate disulfide or corresponding thiolate anion. Suitable cytokines include, e.g., interferon α, interferon β, interferon γ, and interleukin 6 (IL-6). Such cytokines, when administered to a patient, are capable of inducing an acute phase response in the body of the patient, thus stimulating elevations of serum ceruloplasmin in the patient.

The biochemical and physiological properties of such cytokines have been studied extensively in the art and are familiar to skilled artisans. The cytokines can be purified from human or animal serum. They can also be obtained by genetic engineering techniques. In addition, commercially available samples of the above-identified cytokines may also be used in this invention. Genetically or chemically modified cytokines can also be administered. For example, it is known that certain peptidic cytokines have longer circulation time in animals when such cytokines are conjugated with a water soluble, non-immunogenic polymer such as polyethylene glycol.

Typically, the cytokines are administered in a different composition from the dithiocarbamate disulfide or corresponding thiolate anion. The cytokines and dithiocarbamate disulfide or thiolate anion can be administered at about the same time, or at some time apart from each other. For example, the cytokines can be administered from about 5 minutes to about 24 hours before or after the administration of dithiocarbamate disulfide or thiolate anion.

In accordance with another aspect of this invention, the method of this invention can be used in combination with a conventional cancer chemotherapy with the result that the treatment with dithiocarbamate disulfides or thiolate anions, with or without metal ion as dithiocarbamate-metal chelate compounds, will increase the sensitivity of the tumor to conventional cancer chemotherapy and result in greater effectiveness of the conventional cancer chemotherapeutic drug. For example, the method of this invention can be complemented by a conventional radiation therapy or chemotherapy. Thus, in one embodiment of this invention, the method of this invention comprises administering to a patient a dithiocarbamate disulfide compound or corresponding dithiolate metal ion chelate compound, and another anticancer agent. Treatment by ceruloplasmin or a cytokine and a dithiocarbamate disulfide or thiolate anion can also be conducted concurrently with treatment by another anticancer agent to increase the effectiveness of that anticancer agent.

Any anticancer agents known in the art can be used in this invention so long as they are pharmaceutically compatible with the dithiocarbamate disulfide, thiolate anion, metal compound, ceruloplasmin, and/or cytokines used. By “pharmaceutically compatible” it is intended that the other anticancer agent will not interact or react with the above composition directly or indirectly in such a way as to adversely affect the effect of the treatment of cancer, or to cause any significant adverse side reaction to the patient.

Exemplary anticancer agents known in the art include busulphan, chlorambucil, hydroxyurea, ifosfamide, mitomycin, mitotane, mechlorethamine, carmustine, lomustine, cisplatin, herceptin, carboplatin, cyclophosphamide, nitrosoureas, fotemustine, vindescine, etoposide, daunorubicin, adriamycin, paclitaxel, docetaxel, streptozocin, dactinomycin, doxorubicin, idarubicin, plicamycin, pentostatin, mitotoxantrone, valrubicin, cytarabine, fludarabine, floxuridine, clardribine, methotrexate, mercaptopurine, thioguanine, capecitabine, irinotecan, dacarbazine, asparaginase, gemcitabine, altretamine, topotecan, procarbazine, vinorelbine, pegaspargase, vincristine, rituxan, vinblastine, tretinoin, teniposide, fluorouracil, melphalan, bleomycin, salicylates, aspirin, piroxicam, ibuprofen, indomethacin, naprosyn, diclofenac, tolmetin, ketoprofen, nambuetone, oxaprozin, doxirubicin, nonselective cyclooxygenase inhibitors such as nonsteroidal anti-inflammatory agents (NSAIDs), selective cyclooxygenase-2 (COX-2) inhibitors, tamoxifin, and lipooxygenase (LOX) inhibitors.

The anticancer agent used can be administered simultaneously in the same pharmaceutical preparation with the dithiocarbamate disulfide, thiolate anion compound, dithiocarbamate-metal ion chelate compounds, ceruloplasmin, and/or cytokines as described above. The anticancer agent can also be administered at about the same time but by a separate administration. Alternatively, the anticancer agent can be administered at a different time from the administration of the dithiocarbamate disulfide or thiolate anion compound or dithiocarbamate-metal ion coordination compounds, ceruloplasmin, and/or cytokines. Some minor degree of experimentation may be required to determine the best manner of administration, this being well within the capability of one skilled in the art once appraised of the present disclosure.

The methods for treating cancer presented in this invention are particularly useful for treating humans. Also, the methods of this invention are suitable for treating cancers in animals, especially mammals, such as canines, bovines, porcines, and other animals. The methods are useful for treating various types of cancer including, but not limited to, melanoma, non-small cell lung cancer, small cell lung cancer, renal cancer, colorectal cancer, breast cancer, pancreatic cancer, gastric cancer, bladder cancer, ovarian cancer, uterine cancer, lymphoma and prostate cancer. In particular, the present invention will be especially effective in treating melanoma, lung cancer, breast cancer, colon cancer and prostate carcinoma.

The active compounds of this invention are typically administered in a pharmaceutically acceptable carrier through many appropriate routes; for example parenterally, intravenously, orally, intradermally, subcutaneously, or topically, an as described in more detail below. The active compounds of this invention are administered at a therapeutically effective level to achieve the desired therapeutic effect without causing any serious adverse effects in the patient.

The dithiocarbamate disulfide compound disulfiram and its diethyldithiocarbamate anion are effective when administered at amounts within the conventional clinical ranges determined in the art. Disulfiram approved by the U.S. Food and Drug administration (Antabuse®) can be purchased in 250 and 500 mg tablets for oral administration from Odyssey Pharmaceuticals, East Hanover, N.J. 07936. Typically, it is effective at an amount of from about 125 to about 1000 mg per day, preferably from 250 to about 500 mg per day for disulfiram and 100 to 500 mg per day or 5 mg/kg intravenously or 10 mg/kg orally once a week for diethyldithiocarbamate. However, the dosage can vary with the body weight of the patient treated. The active ingredient may be administered at once, or may be divided into a number of smaller doses to be administered at predetermined intervals of time. The suitable dosage unit for each administration of disulfiram is, e.g., from about 50 to about 1000 mg/day, preferably from about 250 to about 500 mg/day. The desirable peak concentration of disulfiram generally is about 0.05 to about 10 μM, preferably about 0.5 to about 5 μM, in order to achieve a detectable therapeutic effect. Similar concentration ranges are desirable for dithiocarbamate thiolate anions and for dithiocarbamate-metal ion chelate compounds.

Disulfiram implanted subcutaneously for sustained release has also been shown to be effective for alcoholism at an amount of 800 to 1600 mg to achieve a suitable plasma concentration. This can be accomplished by using aseptic techniques to surgically implant disulfiram into the subcutaneous space of the anterior abdominal wall. (See e.g., Wilson, et al., J. Clin. Psych. 45:242-247 (1984).) In addition, sustained release dosage formulations, such as an 80% poly(glycolic-co-L-lactic acid) and 20% disulfiram, may be used. The therapeutically effective amount for other dithiocarbamate disulfide compounds may also be estimated or calculated based on the above dosage ranges of disulfiram and the molecular weights of disulfiram and the other dithiocarbamate disulfide compound, or by other methods known in the art.

The diethyldithiocarbamate thiolate anion has not been previously advocated as a cancer chemotherapeutic agent itself, nor has it been suggested as a treatment to increase the sensitivity of tumors to cancer chemotherapy drugs. For the treatment of HIV infection, humans have been treated with doses of 5 mg/kg intravenous or 10 mg/kg orally, once a week. Minimal side effects on this dosage regimen include a metallic taste in the mouth, flatulence, and intolerance to alcoholic beverages. An enteric-coated oral dosage form of diethyldithiocarbamate anions to liberate active drug only in the alkaline environment of the intestine is preferred because of the potential for liberation of carbon disulfide upon exposure of diethyldithiocarbamate to hydrochloric acid in the stomach. An oral enteric-coated form of sodium diethyldithiocarbamate is available in 125 mg tablets as Imuthiol® through Institute Merieux, Lyon, France.

Metal ions can be administered separately as aqueous solutions. In the case of charged metal ion coordination complexes, the metal ions are administered in a pharmaceutically suitable form. Ideally, the charged metal species contains the metal ion coordinated to a chelating agent such as acetate, lactonate, glycinate, citrate, propionate, or gluconate, with a pharmaceutically acceptable counter ion. However, the metal ions are preferably administered with the dithiocarbamate moiety coordinated to the metal ion. Thus, the amount of metal ion to be used is proportional to the amount of dithiocarbamate to be administered based on the stoichiometric ratio between a metal ion and the dithiocarbamate in the complex. Methods for preparing such chelates or complexes are known and the preferred methods are disclosed above and in the examples below.

Traditional chemotherapeutic agents can be utilized in combination with the compounds disclosed herein. Such agents can be-coadministered in amounts known to those skilled in the art. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

Advantageously, the active compounds are delivered to the patient parenterally, i.e., intravenously or intramuscularly. For parenteral administration, the active compounds can be formulated into solutions or suspensions, or in lyophilized forms for conversion into solutions or suspensions before use. Sterile water, physiological saline, e.g., phosphate buffered saline (PBS) can be used conveniently as the pharmaceutically acceptable carriers or diluents. Conventional solvents, surfactants, stabilizers, pH balancing buffers, anti-bacteria agents, and antioxidants can all be used in the parenteral formulations, including but not limited to acetates, citrates or phosphate buffers, sodium chloride, dextrose, fixed oils, glycerin, polyethylene glycol, propylene glycol, benzyl alcohol, methyl parabens, ascorbic acid, sodium bisulfite, and the like. For parenteral administration; the active compounds, particularly dithiocarbamate-metal chelates, can be formulated contained in liposomes so as to enhance absorption and decrease potential toxicity. The parenteral formulation can be stored in any conventional containers such as vials, ampoules, and syringes.

The active compounds can also be delivered orally in enclosed capsules or compressed tablets. Capsules and tablets can be prepared by any conventional techniques. For example, the active compounds can be incorporated into a formulation that includes pharmaceutically acceptable carriers such as excipients (e.g., starch, lactose), binders (e.g., gelatin, cellulose, gum), disintegrating agents (e.g., alginate, Primogel, and corn starch), lubricants (e.g., magnesium stearate, silicon dioxide), and sweetening or flavoring agents (e.g., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules. For administration of dithiocarbamate thiolate anions and dithiocarbamate-metal compounds, it is desirable to administer the compounds as enteric-coated capsules that are impervious to stomach acid but dissolve in the alkaline environment of the small intestine, in order to prevent release of carbon disulfide from dithiocarbamates in the acid environment of the stomach, and to preserve the integrity of the dithiocarbamate metal compound.

Other forms of oral formulations such as chewing gum, suspension, syrup, wafer, elixir, and the like can also be prepared containing the active compounds used in this invention. Various modifying agents for flavors, tastes, colors, and shapes of the special forms can also be included. In addition, for convenient administration by enteral feeding tube in patients unable to swallow, the active compounds can be dissolved in an acceptable lipophilic vegetable oil vehicle, such as olive oil, corn oil, and safflower oil.

The active compounds can also be administered topically through rectal, vaginal, nasal or mucosal applications. Topical formulations are generally known in the art including creams, gels, ointments, lotions, powders, pastes, suspensions, sprays, and aerosols. Typically, topical formulations include one or more thickening agents, humectants, and/or emollients including but not limited to xantham gum, petrolatum, beeswax, or polyethylene glycol, sorbitol, mineral oil, lanolin, squalene, and the like. A special form of topical administration is delivery by a transdermal patch. Methods for preparing transdermal patches are disclosed, e.g., in Brown, et al., Annual Review of Medicine. 39:221-229 (1988), which is incorporated herein by reference.

The active compounds can also be delivered by subcutaneous implantation for sustained release. This may be accomplished by using aseptic techniques to surgically implant the active compounds in any suitable formulation into the subcutaneous space of the anterior abdominal wall. (See e.g., Wilson, et al., J. Clin. Psych. 45:242-247 (1984).) Sustained release can be achieved by incorporating the active ingredients into a special carrier such as a hydrogel. Typically, a hydrogel is a network of high molecular weight biocompatible polymers, which can swell in water to form a gel like material. Hydrogels are generally known in the art. For example, hydrogels made of polyethylene glycols, or collagen, or poly(glycolic-co-L-lactic acid) are suitable for this invention. (See e.g., Phillips, et al., J. Pharmceut. Sci. 73:1718-1720 (1984).)

The active compounds can also be conjugated, i.e., covalently linked, to a water soluble non-immunogenic high molecular weight polymer to form a polymer conjugate. Advantageously, such polymers, e.g., polyethylene glycol, can impart solubility, stability, and reduced immunogenicity to the active compounds. As a result, the active compound in the conjugate when administered to a patient, can have a longer half-life in the body, and exhibit better efficacy. PEGylated proteins are currently being used in protein replacement therapies and for other therapeutic uses. For example, PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL).

Alternatively, other forms of controlled release or protection including microcapsules and nanocapsules generally known in the art, and hydrogels described above can all be utilized in oral, parenteral, topical, and subcutaneous administration of the active compounds.

As discussed above, another preferable delivery form is using liposomes or encochleates as a carrier. Liposomes are micelles formed from various lipids such as cholesterol, phospholipids, fatty acids and derivatives thereof. Active compounds can be enclosed within such micelles. Methods for preparing liposomal suspensions containing active ingredients therein are generally known in the art and are disclosed in, e.g., U.S. Pat. No. 4,522,811, which is incorporated herein by reference. Several anticancer drugs delivered in the form of liposomes are known in the art and are commercially available from Liposome, Inc., of Princeton, N.J. It has been shown that liposomal delivery can reduce the toxicity of the active compounds, and increase their stability.

The active compounds can also be administered in combination with other active agents that treat or prevent another disease or symptom in the patient treated. However, it is to be understood that such other active agents should not interfere with or adversely affect the effects of the active compounds of this invention on the cancer being treated. Such other active agents include but are not limited to antiviral agents, antibiotics, antifungal agents, anti-inflammation agents, antithrombotic agents, cardiovascular drugs, cholesterol lowering agents, hypertension drugs, and the like.

It is to be understood that individuals placed on dithiocarbamate, disulfide, or thiolate anion therapy for their cancer in any form must be warned against exposure to alcohol in any form, to avoid the precipitation of nausea and vomiting from buildup of acetaldehyde in the bloodstream. Subjects therefore must not only refrain from ingesting alcohol containing beverages, but should also not ingest over the counter formulations such as cough syrups containing alcohol or even use rubbing alcohol topically.

As described above, the compounds can be administered, for example, orally, parenterally, (IV, IM, depo-IM, SQ, and depo SQ), sublingually, intranasally (inhalation), intrathecally, topically, or rectally. Dosage forms known to those of skill in the art are suitable for delivery of the compounds.

Compositions are provided that contain therapeutically effective amounts of the compounds. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

Pharmaceutically acceptable excipients, diluents, solubilizers, solvents, adjuvants and carriers generally include, by way of non-limiting example, mannitol, lactose, starches, gum arabic, calcium silicate, microcrystalline cellulose, polyvinylpyrrolidone, cellulose, water, syrup, and methyl cellulose, lubricating agents such as, for example, talc, magnesium stearate and mineral oil; wetting agents; emulsifying and suspending agents; preserving agents such as methyl- and propyl hydroxybenzoates; sweetening agents; or flavoring agents; polyols, buffers, and inert fillers; mannitol, sorbitol, xylitol, sucrose, maltose, glucose, lactose, dextrose, and the like; buffers including phosphate, citrate, tartrate, succinate, and the like; inert fillers; bulking agents and/or granulating agents.

Other non-limiting examples of pharmaceutically acceptable excipients, diluents, solubilizers, solvents, adjuvants and carriers generally include emulsifiers, albumin, gelatin, detergents (e.g., Tween 20, Tween 80, Pluronic F68, bile acid salts), glycerol, polyethylene glycerol, anti-oxidants (e.g., ascorbic acid, sodium metabisulfite), preservatives (e.g., Thimerosal, benzyl alcohol, parabens), bulking substances or tonicity modifiers (e.g., lactose, mannitol), polyethylene glycol, polylactic acid, polglycolic acid, hydrogels, liposomes, microemulsions, micelles, unilamellar or multilamellar vesicles, erythrocyte ghosts, or spheroplasts; fatty acids, waxes, oils, poloxamers and poloxamines.

Still other non-limiting examples of pharmaceutically acceptable excipients, diluents, solubilizers, solvents, adjuvants and carriers generally include lactose, sucrose, or cornstarch in combination with binders like acacia, cornstarch, gelatin, or with disintegrating agents such as cornstarch, potato starch, alginic acid, or with a lubricant like stearic acid or magnesium stearate, vegetable or animal oils such as sunflower oil or fish-liver oil, sterile liquids such as water and oils, with or without the addition of a surfactant and other pharmaceutically acceptable adjuvants, petroleum, animal, vegetable, or synthetic origin, peanut oil, soybean oil, or mineral oil, saline, aqueous dextrose and related sugar solutions, and glycol.

Non-limiting examples of pharmaceutically acceptable carriers are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

About 1 to 1000 mg of a compound or mixture of compounds or a physiologically acceptable species is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit doses of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as, but not limited to, sucrose or saccharin; and a flavoring agent such as, but not limited to, peppermint, methyl salicylate, or fruit flavoring.

When the dosage unit form is a capsule, it can contain, in addition to material of the above type, a liquid carrier such as fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required or desired.

Where administered intravenously, suitable carriers include physiological saline, phosphate buffered saline (PBS), and solutions containing thickening and solubilizing agents such as glucose, polyethylene glycol, polypropylene glycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds.

Compounds may be administered enterally or parenterally. When administered orally, compounds can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily.

The oral dosage forms are administered to the patient 1, 2, 3, or 4 times daily. It is preferred that the compounds be administered either three or fewer times, more preferably once or twice daily. Hence, it is preferred that the compounds be administered in oral dosage form. It is preferred that whatever oral dosage form is used, that it be designed so as to protect the compounds from the acidic environment of the stomach. Enteric coated tablets are well known to those skilled in the art. In addition, capsules filled with small spheres each coated to protect from the acidic stomach, are also well known to those skilled in the art.

When administered orally, it is preferred that the oral dosage is from about 1 mg/day to about 1000 mg/day. It is more preferred that the oral dosage is from about 25 mg/day to about 500 mg/day. It is understood that while a patient may be started at one dose, that dose may be varied over time as the patient's condition changes.

The compounds can be administered parenterally, for example, by IV, IM, depo-IM, SC, or depo-SC. When administered parenterally, a therapeutically effective amount of about 1 to about 1000 mg/day, preferably from about 25 to about 500 mg daily should be delivered. When a depot formulation is used for injection once a month or once every two weeks, the dose should be about 1 mg/day to about 1000 mg/day, or a monthly dose of from about 3000 mg to about 30,000 mg.

The compounds can be administered sublingually. When given sublingually, the compounds should be given one to four times daily in the amounts described above for IM administration.

The compounds can be administered intranasally. When given by this route, the appropriate dosage forms are a nasal spray or dry powder, as is known to those skilled in the art. The dosage of the compounds for intranasal administration is the amount described above for IM administration.

The compounds can be administered intrathecally. When given by this route the appropriate dosage form can be a parenteral dosage form as is known to those skilled in the art. The dosage of the compounds for intrathecal administration is the amount described above for IN administration.

The compounds can be administered topically. When given by this route, the appropriate dosage form is a cream, ointment, or patch. Because of the amount of the compounds to be administered, the patch is preferred. When administered topically, the dosage is from about 1 mg/day to about 1000 mg/day. Because the amount that can be delivered by a patch is limited, two or more patches may be used. The number and size of the patch is not important, what is important is that a therapeutically effective amount of the compounds be delivered as is known to those skilled in the art. The compounds can be administered rectally by suppository as is known to those skilled in the art. When administered by suppository, the therapeutically effective amount is from about 1 mg/day to about 1000 mg/day.

The compounds can be administered by implants as is known to those skilled in the art. When administering a compound by implant, the therapeutically effective amount is the amount described above for depot administration.

The invention here is the new compounds and new methods of using the compounds. Given a particular compound and a desired dosage form, one skilled in the art would know how to prepare and administer the appropriate dosage form.

It should be apparent to one skilled in the art that the exact dosage and frequency of administration will depend on the particular compounds administered, the particular condition being treated, the severity of the condition being treated, the age, weight, general physical condition of the particular patient, and other medication the individual may be taking as is well known to administering physicians who are skilled in this art.

By “alkyl” and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is shorter or longer than 6 carbons, it will be so indicated in the second “C” as, for example, “C₁-C₁₀” indicates a maximum of 10 carbons.

By “heteroalkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl, wherein 1-3 carbon atoms is substituted or replaced with a heteroatom, such as oxygen, nitrogen or sulfur. It is understood that in cases where an alkyl chain of a substituent (e.g. of an alkyl, alkoxy or alkenyl group) is shorter or longer than 6 carbons, it will be so indicated in the second “C” as, for example, “C₁-C₁₀” indicates a maximum of 10 carbons, wherein.

By “alkoxy” and “C₁-C₆ alkoxy” in the present invention is meant straight or branched chain alkyl groups having 1-6 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexoxy, and 3-methylpentoxy.

By the term “halogen” in the present invention is meant fluorine, bromine, chlorine, and iodine.

“Alkenyl” and “C₂-C₆ alkenyl” means straight and branched hydrocarbon groups having from 2 to 6 carbon atoms and from one to three double bonds and includes, for example, ethenyl, propenyl, 1-but-3-enyl, 1-pent-3-enyl, 1-hex-5-enyl and the like.

“Alkynyl” and “C₂-C₆ alkynyl” means straight and branched hydrocarbon groups having from 2 to 6 carbon atoms and one or two triple bonds and includes ethynyl, propynyl, butynyl, pentyn-2-yl and the like.

As used herein, the term “cycloalkyl” refers to saturated carbocyclic groups having three to twelve carbon atoms. The cycloalkyl can be monocyclic, or a polycyclic fused system. Examples of such groups include cyclopropyl, cyclobutyl, cyclopentyl and cyclohexyl. Preferred cycloalkyl groups are cyclopentyl, cyclohexyl, and cycloheptyl. The cycloalkyl groups herein are unsubstituted or, as specified, substituted in one or more substitutable positions with various groups. For example, such cycloalkyl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), which is optionally mono-, di-, or trisubstituted. Preferred aryl groups of the present invention are phenyl, 1-naphthyl, 2-naphthyl, indanyl, indenyl, dihydronaphthyl, tetralinyl or 6,7,8,9-tetrahydro-5H-benzo[α]cycloheptenyl. The aryl groups herein are unsubstituted or, as specified, substituted in one or more substitutable positions with various groups. For example, such aryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆ alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heteroaryl” is meant one or more aromatic ring systems of 5-, 6-, or 7-membered rings which include fused ring systems of 9-11 atoms containing at least one and up to four heteroatoms selected from nitrogen, oxygen, or sulfur. Preferred heteroaryl groups of the present invention include pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide and benzothiopyranyl S,S-dioxide. The heteroaryl groups herein are unsubstituted or, as specified, substituted in one or more substitutable positions with various groups. For example, such heteroaryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆ alkenyl, C₂-C₆ alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heterocycle”, “heterocycloalkyl” or “heterocyclyl” is meant one or more carbocyclic ring systems of 4-, 5-, 6-, or 7-membered rings which includes fused ring systems of 9-11 atoms containing at least one and up to four heteroatoms selected from nitrogen, oxygen, or sulfur. Preferred heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. The heterocycle groups herein are unsubstituted or, as specified, substituted in one or more substitutable positions with various groups. For example, such heterocycle groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆ alkenyl, C₂-C₆ alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

The following abbreviations are defined as used herein: NF-KB, nuclear factor-KB; 5-FU, 5-fluorouracil; SOD, superoxide dismutase; Cu, copper(II); Zn, zinc (II); EDTA, ethylenediaminetetraacetic acid; HEPES, N-2-hydroxyethylpiperazine-N′-2-ethanesulfonic acid; FBS, fetal bovine serum; CRE, cyclic AMP responsive element; DS, disulfiram; DMSO, dimethylsulfoxide; MTT, 3-[4,5-dimethylthiazol]-2yl-2,5-diphenyl tetrazolium bromide; DPBS, Dulbecco's phosphate buffered saline; NAC, N-acetylcysteine; GSH, glutathione.

### Experimental Procedures

Cells

Human malignant cell lines were obtained from American Type Tissue Culture Collection (Rockville, Md.). Melanoma cells lines CRL 1585 and 1619 were cultured in RPMI 1640 (GIBCO-BRL, Life Technologies, Grand Island, N.Y.) with 10% fetal bovine serum (FBS) and passed with nonenzymatic Cell Dissociation Solution (Sigma). The prostate adenocarcinoma cell line CRL 1435 (PC-3) and the ovarian cancer cell lines HTB75 and HTB77 were also cultured in RPMI 1640 with 10% FBS but passed with 0.05% trypsin and 0.53 mM ethylenediaminetetraacetic acid (EDTA). The squamous lung carcinoma NCI-H520 and the adenosquamous lung carcinoma NCI-H596 cell lines were grown in RPMI 1640 supplemented with 10% FBS, 10 mM N-2-hydroxyethylpiperazine-N′-2-ethanesulfonic acid (HEPES) and 1.0 mM sodium pyruvate and passed with trypsin/EDTA. All of the above were grown in a 37° C. humidified environment containing 5% CO₂/air. The breast carcinoma cell line MDA-MB-453 was grown in a 37° C. humidified environment with free atmospheric gas exchange, Leibovitz's L-15 medium with 2 mM L-glutamine and 10% FBS, and was passed with trypsin/EDTA.

Cell Treatments

As others have suggested that the disulfide form of dithiocarbamates is the active proximate chemical form that mediates mixed disulfide formation with protein thiols (see, e.g., Burkitt M J, Bishop H S, Milne L, et al. Dithiocarbamate toxicity toward thymocytes involves their copper-catalyzed conversion to thiuram disulfides, which oxidize glutathione in a redox cycle without the release of reactive oxygen species. Arch Biochem Biophys, 1998; 353:73-84; Nobel C S I, Burgess D H, Zhivotovsky B, et al. Mechanism of dithiocarbamate inhibition of apoptosis: thiol oxidation by dithiocarbamate disulfides directly inhibits processing of the caspase-3 proenzyme. Chem Res Toxicol, 1997; 10:636-643; Balakirev M Y, Zimmer G. Mitochondrial injury by disulfiram: two different mechanisms of the mitochondrial permeability transition. Chem-Biol Interact, 2001; 1138:299-3110, experiments were conducted with the tetraethylthiuram disulfide disulfiram (Sigma), which does not have a free thiol to act as an antioxidant. Malignant melanoma cells grown to confluence on 100×15 mm plastic Petri dishes were treated with 0-5 μM disulfiram. These doses were chosen to approximate the steady state plasma and tissue concentrations previously reported in humans treated with disulfiram (see, Faiman M D, Jensen J C, Lacoursiere R B. Elimination kinetics of disulfiram in alcoholics after single and repeated doses. Clin Pharmacol Ther, 1984; 36:520-526). Without being bound by any particular theory, it is postulated that disulfiram is converted to its bis(diethyldithiocarbamato)copper(II) complex after passage through the acid environment of the stomach. See, Johansson B. A review of the pharmacokinetics and pharmacodynamics of disulfiram and its metabolites. Acta Psychitrica Scand, Suppl, 1992; 369:15-26. Copper(II) was added along with disulfiram in some experiments to stimulate formation of the disulfiram-copper chelate form in which the drug is systemically absorbed. Disulfiram was dissolved in dimethylsulfoxide (DMSO) to a final concentration <0.3-0.5%. Equal volumes of DMSO were added to control experiments.

The effect of disulfiram (0.15 to 5.0 μM) or sodium diethyldithiocarbamate (1.0 μM) on proliferation of malignant cell lines was studied in cultures stimulated with 10% FBS. Cell numbers were quantitated 24-72 hr later as outlined below. In some experiments disulfiram was added immediately after cells were plated. In other experiments, cells were plated and allowed to grow for 24-72 hr before fresh media with disulfiram was added, and cell numbers were assayed 24-72 hr later. Synergy was studied between disulfiram and N,N′-bis(2-chloroethyl-N-nitrosourea (carmustine, 1.0 to 1,000 μM) or cisplatin (0.1 to 100 μg/mL) added to medium. The effect of metal ions on disulfiram was studied with 0.2 to 10 μM copper(II) (provided as CuSO₄), zinc(II) (as ZnCl₂), silver(I) (as silver lactate) or gold(III) (as HAuCl₄3H₂O) ions added to growth medium, buffered to physiologic pH. To provide a biologically relevant source of copper, medium was supplemented with human ceruloplasmin at doses replicating low and high normal adult serum concentrations (250 and 500 mg/mL).

To determine whether disulfiram and metal ions might directly influence transcription factor binding, 5 μM disulfiram and/or 1.6 μM CuSO₄ (final concentrations) were added to the binding reaction of nuclear protein obtained from control cells stimulated with 10% FBS alone in the absence of drugs or metal ions. The binding reaction was then performed using either 2.5 mM dithiothreitol or 3.0 mM glutathione as the buffer reducing agent.

In additional experiments the effect of disulfiram was studied on expression of cyclic AMP responsive element (CRE)-regulated cell cycle proteins and proteins influencing apoptosis. Confluent cells treated with 5 μM disulfiram or 5 μM disulfiram plus 1.6 μM CuSO₄ for 2 to 48 hr. Cells were lysed and levels of the pro-apoptotic protein p53, the anti-apoptotic protein Bcl-2, the cyclin inhibitor p21^{WAFI/Cipl}, and the cyclins A and B1 were measured by immunoblots as described below.

Potential redox effects of disulfiram were studied in three sets of experiments. The importance of cellular glutathione in thiocarbamate toxicity was studied by measuring levels of intracellular glutathione after treatment with disulfiram. Confluent monolayers were treated with disulfiram (5 μM), with or without 1.6 μM CuSO₄, and cells were harvested 24 hr later for measurement of glutathione. To assess whether a pro-oxidant effect of disulfiram accounts for growth inhibition, we studied the effect of the potent lipophilic antioxidant probucol (1.0 to 1,000 μM) on disulfiram's anti-proliferative effect. Finally, generation of intracellular oxidants in response to disulfiram (0.625 to 5 μM), copper(II) (0.2 to 1.6 μM CuSO₄) or 1.25 μM disulfiram plus various concentrations of copper(II) was measured directly, as outlined below.

Dithiocarbamates have been reported in the art to inhibit proliferation of malignant cells by reducing cyclooxygenase-2 production of mitogenic prostaglandins. See, Chinery R, Beauchamp R D, Shyr Y, et al. Antioxidants reduce cyclooxygenase-2 expression, prostaglandin production, and proliferation in colorectal cancer cells. Cancer Res, 1998; 58:2323-2327. To explore the role of cyclooxygenase inhibition on tumor growth, cells were cultured with or without disulfiram in the presence or absence of the cyclooxygenase-1 and cyclooxygenase-2 inhibitors indomethacin (5 μg/mL) or sodium salicylate (1 mM). Dithiocarbamates have also been shown in the art to increase cytoplasmic levels of nitric oxide (NO.) by decomposing S-nitrosoglutathione. See, Arnelle D R, Day B J, Stamler J S. Diethyl dithiocarbamate-induced decomposition of S-nitrosothiols. Nitric Oxide: Biol and Chem, 1997; 1:56-64. Without being bound by any particular theory, NO. could, in turn, induce mitochondrial permeability transition and apoptosis. To probe whether disulfiram might be inducing growth retardation by altering NO. production, proliferation was studied with and without disulfiram in the presence and absence of the nitric oxide synthase inhibitor Nω-nitro-L-arginine added to growth medium (100 μM).

Finally, a number of dithiocarbamate effects have been attributed in the art to increasing the intracellular levels of copper ions. See, Erl W, Weber C, Hansson G K. Pyrrolidine dithiocarbamate-induced apoptosis depends on cell type, density, and the presence of Cu(II) and Zn(II). Am J Physiol Cell Physiol, 2000; 278:C116-C1125; and Verhaegh G W, Richard M-J, Hainaut P. Regulation of p53 by metal ions and by antioxidants: Dithiocarbamate down-regulated p53 DNA-binding activity by increasing the intracellular level of copper. Mol Cell Biol, 1997; 17:5966-5706. To further probe the role of copper ions in mediating cytotoxicity from disulfiram, cells were cultured with or without addition of the impermeate Cu(II) chelator bathocuprioinedisulfonic acid (50 or 100 μM) added to medium to sequester Cu(II) in the extracellular compartment. Cells were also treated 12 hours with various concentration of disulfiram (0.625 to 5.0 μM) and intracellular copper levels were measured as outlined below.

Electrophoretic Mobility Shift Assays

Nuclear protein was isolated and DNA binding reactions were performed and quantitated as previously detailed (see, Brar S S, Kennedy T P, Sturrock A B, et al. An NAD(P)H oxidase regulates growth and transcription in melanoma cells. Am J Physiol Cell Physiol, 2002; 282:C1212-C1224) in the art using consensus oligonucleotides (5′-AGAGATTGCCTGACGTCAGAGAGCTAG-3′ and 3′-TCTCTAACGGACTGCAGTCTCTCGATC-5′) for the cyclic-AMP responsive element CRE, and (5′-AGTTGAGGGGACTTTCCCAGGC-3′ and 3′-TCAACTCCCCTGAAAGGGTCCG-5′) for NF-κB (p50) (ProMega, Madison, Wis.). Competition experiments were performed with 10× unlabeled wild-type oligonucleotide sequences for CRE or NF-κB. Supershift experiments were performed by incubating the binding reaction with 1 μg of supershifting antibody (Santa Cruz Biotechnology) prior to electrophoresis.

Measurement of Proliferation in Cell Cultures

Proliferation of cultured cells seeded into 24-well uncoated plastic plates (Costar) at 50,000 cells per well was quantitated as previously detailed (see, Brar S S, Kennedy T P, Whorton A R, et al. Requirement for reactive oxygen species in serum-induced and platelet-derived growth factor-induced growth of airway smooth muscle. J Biol Chem, 1999; 274:20017-20026) in the art using a colorimetric method based upon metabolic reduction of the soluble yellow tetrazolium dye 3-[4,5-dimethylthiazol]-2yl-2,5-diphenyl tetrazolium bromide (MTT) to its insoluble purple formazan by the action of mitochondrial succinyl dehydrogenase. This assay was confirmed by experiments in which cells were stained with Wright's modified Giemsa, counterstained with eosin and counted directly at a magnification of 100× using a 1-mm² ocular grid.

Measurement of Apoptosis

Apoptosis was studied by terminal deoxynucleotidyl transferase (TdT) dependent 3′-OH fluorescein end-labeling of DNA fragments, using a Fluorescein-FragEL™ DNA fragmentation detection kit (Oncogene Research Products, Cambridge, Mass.), by fluorescent-labeled annexin V staining of phosphatidylserine translocated to the membrane surface, using the Annexin-V FLUOS staining kit (Roche Molecular Biochemical, Indianapolis, Ind.), and by visually assessing endonuclease dependent DNA fragmentation on ethidium bromide-stained agarose gels.

DNA Cell Cycle Measurements

To study the effect of disulfiram on the DNA cell cycle, confluent cells were treated with 10% FBS plus DMSO vehicle, FBS and DMSO vehicle plus 250 mg/mL ceruloplasmin as a source of copper(II), FBS plus 5 μM disulfiram or FBS plus 5 μM disulfiram and 250 mg/mL ceruloplasmin. After 24 hrs cells were trypsinized, washed twice in cold Dulbecco's phosphate buffered saline (DPBS) with 1 mM EDTA and 1% BSA, fixed 30 min in ice-cold 70% ethanol, and stained by incubation for 30 min at 37° C. in a 10 mg/mL solution of propidium iodide in DPBS and 1 mg/mL RNase A. DNA cell cycle measurements were made using a FACStar^{PLUS} Flow Cytometer (Becton-Dickinson, San Jose, Calif.).

Immunoblots for Proteins

Immunoblots were performed and quantitated as described previously (22) using primary rabbit polyclonal antibodies against human bcl-2, p53, p21^{WAFI/Cipl}, cyclin A and cyclin B1, and peroxidase-labeled donkey polyclonal anti-rabbit IgG (Santa Cruz).

Measurement of Intracellular Copper

Cells were cultured in 12-well plastic tissue culture plates at an initial plating density of 50,000 cells/well, grown to confluence and treated with disulfiram or DMSO vehicle as outlined above. Media was removed and cells were washed twice with DPBS. Cells were then scraped into 1.0 mL of 3N HC1/10.0% trichloroacetic acid and hydrolyzed at 70° C. for 16 hr. The hydrolysate was centrifuged at 600 g for 10 min to remove debris and copper was measured in the supernatant using inductively coupled plasma emission spectroscopy (Model P30, Perkin Elmer, Norwalk, Conn.) at wavelengths of 325.754 and 224.700 nm. To minimize metal contamination, plasticware rather than glassware was used in these experiments, and double-distilled, deionized water was used for all aqueous media. Results are reported as ng copper/culture well.

Measurement of Intracellular Generation of Reactive Oxygen Species

Generation of reactive oxygen species in response to disulfiram with or without CuSO₄ was studied using 2′,7′-dichlorofluorescin diacetate (Molecular Probes, Eugene, Oreg.) and a modification of methods previously reported in the art. See, Ubezio P, Civoli F. Flow cytometric detection of hydrogen peroxide production induced by doxorubicin in cancer cells. Free Rad Biol Med, 1994; 16:590-516. Cells were plated in 24 well plastic plates at 50,000 cells per well and grown to confluence. Media was aspirated from wells and replaced with 100 μL medium containing 10 μM dichlorofluorescin diacetate, and plates were incubated at 37° C. for 30 min. The dichlorofluorescin diacetate containing media was aspirated, cells were washed twice with media alone and 100 μL fresh media was added to wells. With the plate on the fluorescence micro-plate reader (HTS 7000) cells were stimulated with 25 μL of media containing 5× concentrations of disulfiram and/or CuSO₄ to provide final concentrations of 0-5.0 μM disulfiram and/or 0-1.6 μM CuSO₄, respectively. The relative concentration of dichlorofluroescein was measured immediately by monitoring fluorescence at 37° C. using an excitation wavelength of 485 nm and emission wavelength of 535 nm.

Measurement of Intracellular Glutathione

Disulfiram (5 μM, with or without 1.6 μM CuSO₄, was added to cells grown to confluence on 100×15 mm plastic dishes, and cells were harvested 24 hr later for measurement of glutathione using the 5,5′-dithiobis(2-nitrobenzoic acid)-glutathione reductase recycling assay. See, Anderson M E. Determination of glutathione and glutathione disulfide in biological samples. Methods Enzymol, 1985; 113:548-555.

Additional General Synthesis of Dithiocarbamate-Metal Chelates

Synthesis of diethyldithiolato metal complexes is known in the literature. Typically, aqueous solutions of a metal salt, e.g., CuCl₂, and sodium or ammonium diethyldithiocarbamate are mixed and the desired complex separated by extraction into an organic phase such as dichloromethane. The stoichiometric ratio between metal species and diethyldithiocarbamate ligand can influence the final stoichiometry of the product. Identical complexes were synthesized starting with disulfiram rather than diethyldithiocarbamate. All diethyldithiocarbamato metal complexes were characterized by means of a single crystal X-ray diffraction.

Study of Anti-Tumor Activity of Disulfiram and Zinc Supplementation In Vivo

Adult female CB17-SCID mice (Harlan, Indianapolis, Ind.) were housed in a protected laminar flow facility with access to water and either a standard diet containing 87 ppm zinc or a zinc supplemented diet (Harlan) containing 1,000 ppm zinc(II) as zinc acetate. Mice were injected subcutaneously in the right groin with 5×10⁶ cells from a highly aggressive malignant melanoma obtained from a Carolinas Medical Center patient. The frozen tumor was passaged twice in SCID mice to adapt it to in vivo growth before use in these experiments. On the day of tumor injection all mice began daily administration of drug. Drug was administered in a total volume of 0.2 mL by gastric gavage via smooth Teflon-tipped needles inserted trans-orally into the stomach. Four groups were studied: Tumor Control (n=10; 0.2 mL olive oil daily; zinc diet of 87 ppm); Zinc-Supplemented Control (n=10; 0.2 mL olive oil daily; zinc diet of 1,000 ppm); Disulfiram (n=10; disulfiram 200 mg/kg/day in 0.2 mL olive oil; zinc diet of 87 ppm); and Zinc-Supplemented Diet+Disulfiram (n=10; disulfiram 200 mg/kg/day in 0.2 mL olive oil; zinc diet of 1,000 ppm). Mice were examined daily, the tumor was measured in two dimensions and the tumor volume was estimated using the formula for an ellipse. When estimated tumor volume approached 500 mm³ within any animal, all mice were euthanized. This protocol was reviewed and approved by the Institutional Animal Care and Use Committee at Carolinas Medical Center. Tumors were excised, weighed, fixed in formalin, sectioned and stained with hematoxylin and eosin or immunostained for factor VIII. Slides were coded and examined by a blinded observer who identified vessels as deposits of red cells. For each slide, the number of vessels were counted in four different fields, representative of the tumor. The average number of vessels per field was averaged per biopsy specimen and used to evaluate tumor vascularity.

Results

Disulfiram Inhibits Melanoma Proliferation in a Metal-Dependent Fashion

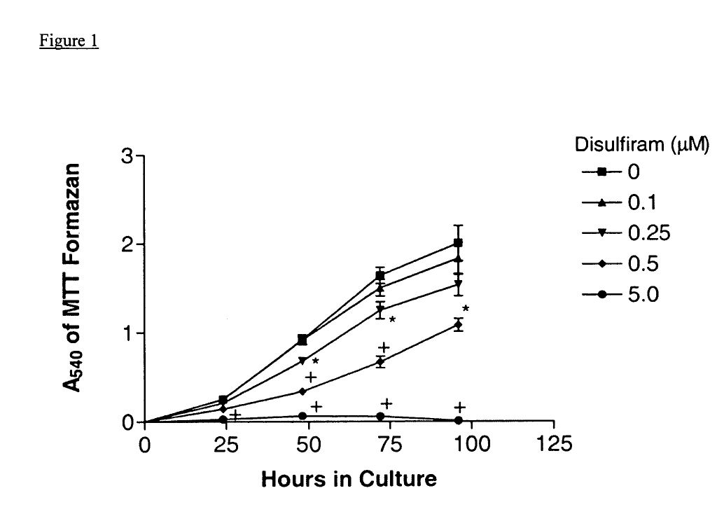

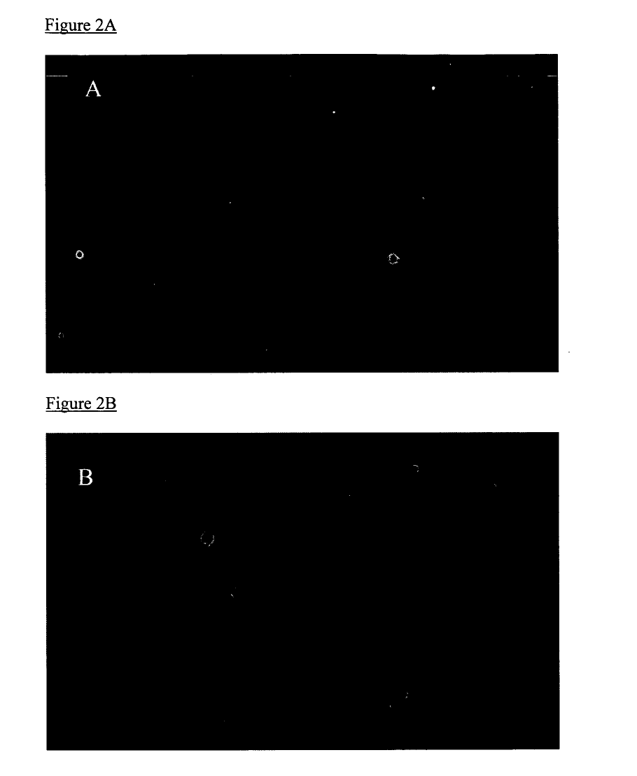

In concentrations reported in humans (see, Faiman M D, Jensen J C, Lacoursiere R B. Elimination kinetics of disulfiram in alcoholics after single and repeated doses. Clin Pharmacol Ther, 1984; 36:520-526), disulfiram inhibited melanoma proliferation in vitro in a dose-dependent fashion, with near complete growth inhibition at 5 μM(p<0.001) (FIG. 1: Cells stimulated with 10% fetal bovine serum (FBS) were plated at a density of 50,000 cells per well, and DMSO vehicle (5 μL per mL) or disulfiram (DS) was added to wells at the indicated concentrations. After 24, 48, 72 or 96 hr, proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance-at 540 nm (A₅₄₀). Two-way analysis of variance shows p<0.001 for group, time and group-time interaction. *p<0.01 at similar culture time versus DMSO vehicle; +p<0.001 at similar culture time point versus DMSO vehicle.), and increased the number of apoptotic cells in culture (FIGS. 2A and 2B: Cells were grown to confluence on 35 mm Petri dishes or on glass slides and treated for 12 hr with disufiram or DMSO as vehicle. Apoptosis was studied by terminal deoxynucleotidyl transferase (TdT) dependent 3′-OH fluorescein end-labeling of DNA fragments, using a Fluorescein-FragEL™ DNA fragmentation detection kit (Oncogene Research Products, Cambridge, Mass.)). Within the same concentration ranges, disulfiram likewise inhibited growth of other malignant cells (See Table 2: 50% inhibitory concentrations=CRL1585 melanoma, 2.5 μM; PC-3 prostate adenocarcinoma, 2.5 μM; H520 squamous cell lung cancer, 0.625 μM; H596 adenosquamous cell lung cancer, 1.25 μM; and MDA-MB-453 breast carcinoma, 0.625 μM). Disulfiram also augmented the antiproliferative effect of cisplatin or carmustine on melanoma cells (See Table 3: 4±1% inhibition of growth at 24 hr with 100 ng/mL cisplatin alone vs 17±3% inhibition with cisplatin and 2.5 μM disulfiram, p<0.05; 46±7% stimulation of growth at 24 hr with 10 μM carmustine alone vs 75±6% inhibition of growth with carmustine and 0.6 μM disulfiram, p<0.001), suggesting that it might reduce resistance to chemotherapy, as recently reported. See Table 2 and Table 3. See also, Loo T W, Clarke D M. Blockage of drug resistance in vitro by disulfiram, a drug used to treat alcoholism. J Natl Cancer Inst, 2000; 92:898-902; and Wang W, McLeod H L, Cassidy J. Disulfiram-mediated inhibition of NF-κB activity enhances cytotoxicity of 5-fluorouracil in colorectal cancer cell lines. Int J Cancer, 2003;104:504-511.

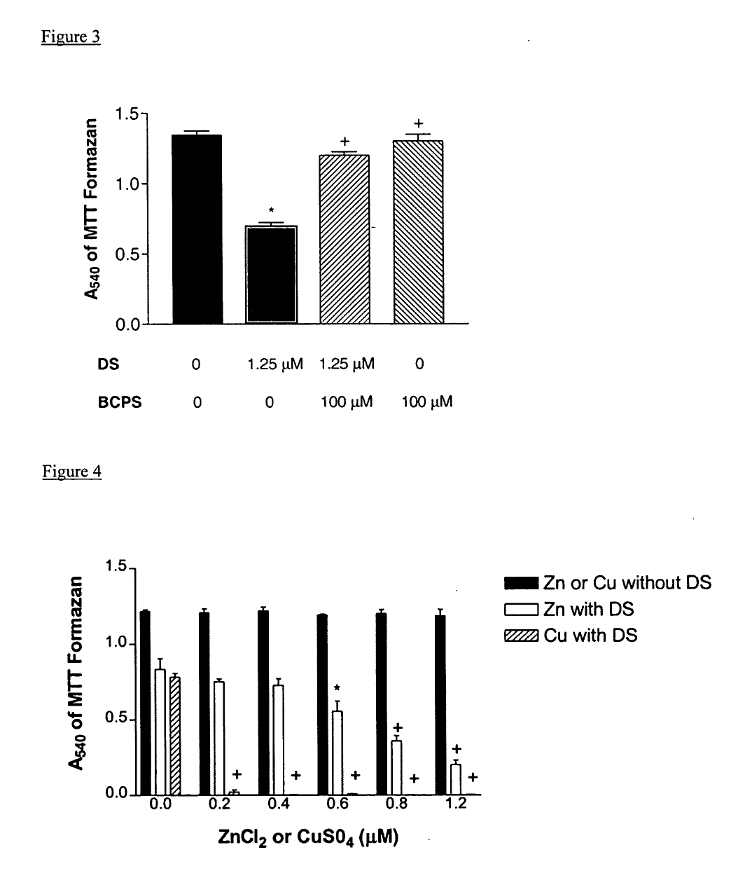

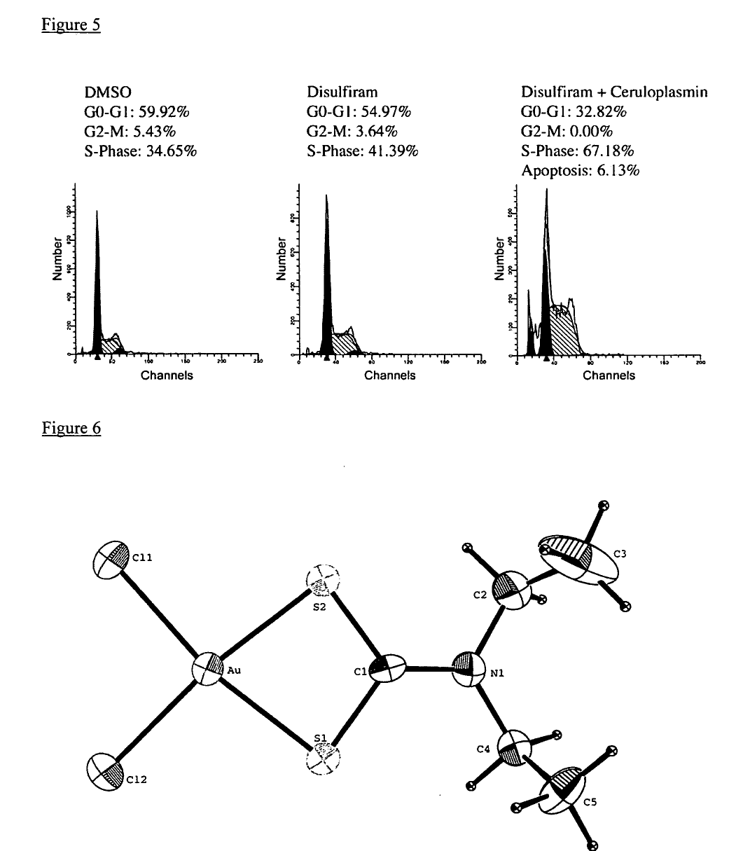

Because dithiocarbamates chelate metals (see, Nobel C S I, Kimland M, Lind B, et al. Dithiocarbamates induce apoptosis in thymocytes by raising the intracellular level of redox-active copper. J Biol Chem, 1995; 270:26202-26208), we explored whether growth inhibition was contingent on disulfiram's ability to complex with metal ions from growth medium. Disulfiram increased intracellular copper in melanoma monolayers (ng copper per well: control=56±7; DMSO vehicle=52±4; 1.25 μM disulfiram=102±5; 2.5 μM disulfiram=160±17; 5.0 μM disulfiram=195±3; all p<0.01 vs control or vehicle). See Table 4. Adding the cell impermeate Cu(II) chelator bathocuproine disulfonic acid (BCPS) to growth medium reversed the antiproliferative activity of disulfiram (FIG. 3: CRL1619 human melanoma cells stimulated with 10% fetal bovine serum (FBS) were plated at a density of 50,000 cells per well and treated with concentrations shown of DMSO vehicle (5 μL/mL) or disulfiram (DS, 1.25 μM with or without the cell impermeate Cu(II) chelator bathocuproine disulfonic acid (BCPS) to complex copper(II) and trap it in the extracellular medium. BCPS reversed growth the antiproliferative activity of disulfiram in a dose-dependent manner (% growth inhibition at 48 hr: 48±2% with 1.25 μM disulfiram; 11±2% with disulfiram+100 μM BCPS; 3±3% with 100 μM BCPS alone)*p<0.001 versus untreated; +p<0.001 versus disulfiram)). Conversely, growth inhibition was enhanced by supplementing medium with copper ion concentrations that do not by themselves affect cell growth (FIG. 4: CRL1619 human melanoma cells stimulated with 10% fetal bovine serum (FBS) were plated at a density of 50,000 cells per well and treated with concentrations shown of DMSO vehicle (5 μL/mL) or disulfiram (DS, 0.625 μM) and concentrations shown of CuSO₄, ZnCl₂, or metal ions plus DMSO or disulfiram. After another 24 hr proliferation was quantitated as in FIG. 1. Addition of even 0.2 μM CuSO₄ to medium converts 0.625 μM disulfiram from a 50% inhibitory (IC₅₀) concentration into a 100% inhibitory (IC₁₀₀) concentration of drug. *p<0.01 and +p<0.001 compared to no CuSO₄ or ZnCl₂.). Ovarian and lung cancer cell lines exhibited similar reversal of disulfiram-induced growth inhibition with BCPS and enhancement of disulfiram-induced growth inhibition by copper ions (See Table 1). In vivo, one potential source of copper ions is the copper transport protein ceruloplasmin which has complexable cupric ions (see, Percival S S, Harris E D. Copper transport from ceruloplasmin: characterization of the cellular uptake mechanism. Am J Physiol, 1990; 258:3140-3146) that could serve as a source of copper to enhance disulfiram. While ceruloplasmin alone has no effect (0±0% growth inhibition with 250 mg/mL human ceruloplasmin), the addition of ceruloplasmin to disulfiram significantly enhances dithiocarbamate-induced growth inhibition (70±2% growth inhibition at 24 hr with 0.625 μM disulfiram; 100±0% growth inhibition with disulfiram+ceruloplasmin, p<0.001). Disulfiram treatment of melanoma cells slightly reduces the number of cells in G₀-G₁ and increases the portion in S-phase of the cell cycle. (FIG. 5: Unsynchronized CRL1619 melanoma cells were grown in the presence of DMSO vehicle, 5 μM disulfiram, or 5 μM disulfiram plus 250 mg/mL ceruloplasmin as a source of copper(II). Twenty-four hours later, cells were harvested and flow cytometric cell cycle analysis was performed. The proportion of nuclei in each phase of the cell cycle was determined with MODFIT DNA analysis software. The portion of cells in G₀-G₁ and in G₂-M is shown in red, the portion in S-Phase are hatched and apoptotic cells are displayed in blue. Disulfiram increases the portion of cells in S-Phase. The combination of disulfiram and ceruloplasmin further increases the number of cells in S-Phase, prevents progression into the G₂-M cell cycle and induces apoptosis. Approximately 6% of cells are apoptotic, over two-thirds of cells are in S-Phase, and none are in G₂-M.). Ceruloplasmin greatly magnifies these effects and produces S-phase cell cycle arrest. Thus, the anti-proliferative effect of disulfiram appears co-dependent upon copper(II). Taken together, these results suggest that the inhibitory effect of disulfiram is critically dependent upon the binding of copper ions from the extracellular medium and transporting them as a dithiocarbamate-metal complex into cells.

Treatments that increase intracellular Cu(II) might be expected to enhance generation of reactive oxygen species. However, disulfiram did not deplete glutathione (228±18 in untreated cells; 254±7 in DMSO vehicle controls; 273±11 nM glutathione/μg cell protein for cells with 5 μM disulfiram), and the combination of 5.0 μM disulfiram and 1.6 μM CuSO₄ even increased glutathione (293±16 nM glutathione/μg cell protein; p<0.05 compared to untreated cells). Likewise, neither disulfiram (0.625 to 5 μM), CuSO₄ (0.2-1.6 μM) nor the combination of 1.25 μM disulfiram and 0.2 to 1.6 μM CuSO₄ caused oxidation of dichlorofluorescin. The baseline fluorescence of 1,431±23 units was not increased by any of the treatments. In addition, the antioxidant probucol did not prevent disulfiram from reducing melanoma proliferation. Augmentation of intracellular copper might also increase levels of nitric oxide (NO.) through Cu(II)-mediated decomposition of nitrosothiols. See, Arnelle D R, Day B J, Stamler J S. Diethyl dithiocarbamate-induced decomposition of S-nitrosothiols. Nitric Oxide: Biol and Chem, 1997; 1:56-64. Without being bound by any particular theory, NO. might, in turn, induce mitochondrial permeability transition and apoptosis. See, Hortelano S, Dallaporta B, Zamzami N, et al. Nitric oxide induces apoptosis via triggering mitochondrial permeability transition. FEBS Lett, 1997; 410:373-377. However, while the nitric oxide synthase inhibitor Nω-nitro-L-arginine alone slightly enhanced cellular growth, it did not eliminate the antiproliferative effect of disulfiram. Thus, disulfiram does not affect cellular redox state. Finally, other dithiocarbamates have been postulated to interfere with growth of colorectal carcinoma by reducing expression of cyclooxygenase-2 (18). However, cyclooxygenase inhibitors failed to reduce melanoma growth.

NF-κB inhibition by dithiocarbamates has recently been associated with facilitation of intracellular zinc transport (see, Kim C H, Kim J H, Moon S J, et al. Biphasic effects of dithiocarbamates on the activity of nuclear factor-κB. Europ J Pharmacol, 2000; 392:133-136), and zinc supplementation increases the toxicity of dithiocarbamates for vascular smooth muscle cells. Zinc substantially enhanced the antiproliferative potential of disulfiram against melanoma cells (FIG. 4). See, Erl W, Weber C, Hansson G K. Pyrrolidine dithiocarbamate-induced apoptosis depends on cell type, density, and the presence of Cu(II) and Zn(II). Am J Physiol Cell Physiol, 2000; 278:C116-Cl 125. Dithiocarbamates can also chelate other metals (see, Burns R P, McCullough F P, McAuliffe C A. 1,1-dithiolato complexes of the transition elements. Adv Inorg Chem Radiochem, 1980; 23:211-280), and gold and silver salts also enhanced the antiproliferative activity of disulfiram (% growth inhibition: 45±5% with 0.15 μM disulfiram; 0±0% with 5 μM silver lactate alone; 71±7% with disulfiram+silver lactate, p<0.001; 0±0% with 5 μM gold tetrachloroauric acid alone; 99±1% with disulfiram+gold tetrachloroauric acid, p<0.001). In light of these findings, we synthesized chelates of disulfiram with Au(III), Cu(II), Zn(II), Ag(I), Ga(III) or Fe(III). X-Ray crystallography confirmed the structures as diethyldithiocarbamato complexes of respective metal ions (A Au(III) complex is shown in FIG. 6; complexes were generated as outlined in the examples below. A Nonius Kappa-CCD diffractometer was used to collect X-ray diffraction data. The crystal diffracted well and a data set was collected to 27.50 in θ using Mo Kα radiation (λ=0.71073 Å). Least-squares refinement on the cell parameters revealed an orthorhombic unit cell with a=11.5167(5), b=7.2472(2), c=12.9350(7) Å, and a volume of 1079.6(1) Å³. Examination of the systematic absences showed the space group to be Pnma (#62). The structure was solved by direct methods using SIR92 and revealed the crystal to be dichloro(diethyldithiocarbamato)gold(III). The structure was confirmed by the successful solution and refinement of the 83 independent variables for the 893 reflections [I>σ(I)] to R-factors of 3.3 and 3.2%, with an ESD of 1.499. The gold complex is a square planar coordination complex in which the gold ion and the four coordinated atoms sit on a mirror at (x, 0.25, z). The organic ligand was found to be disordered with the diethylamine substituents occupying two sites related to each other through the mirror plane. This compound inhibited CRL1619 melanoma growth by 81±1% after exposure for 48 hr to a concentration as low as 0.25 μM.). To confirm that the proximate reactive dithiocarbamate structure important for promoting cellular mixed disulfide formation is the thiolate anion generated from fully reduced dithiocarbamates by metals, we compared the anti-proliferative activity of the thiolate sodium diethyldithiocarbamate alone or in the presence of a low concentration of dithiothreitol to promote formation of the fully reduced thioacid. Sodium diethyldithiocarbamate alone (1 μM) decreased melanoma proliferation by 92±2% after 48 hr (p<0.001), but growth was inhibited by only 24±3% (p<0.001) with simultaneous addition of a concentration of dithiothreitol (100 μM), which does not affect proliferation of melanoma cells by itself (0±0%). Thus, the function of metals may be to facilitate formation of the dithiocarbamate anion, which might condense into mixed disulfides with critical protein sulfhydryls. See, Burkitt M J, Bishop H S, Milne L, et al. Dithiocarbamate toxicity toward thymocytes involves their copper-catalyzed conversion to thiuram disulfides, which oxidize glutathione in a redox cycle without the release of reactive oxygen species. Arch Biochem Biophys, 1998; 353:73-84; Nobel C S I, Burgess D H, Zhivotovsky B, et al. Mechanism of dithiocarbamate inhibition of apoptosis: thiol oxidation by dithiocarbamate disulfides directly inhibits processing of the caspase-3 proenzyme. Chem Res Toxicol, 1997; 10:636-643; and Balakirev M Y, Zimmer G. Mitochondrial injury by disulfiram: two different mechanisms of the mitochondrial permeability transition. Chem-Biol Interact, 2001; 1138:299-311.

Disulfiram and Metals Inhibit ATF/CREB DNA Binding and Cyclin A Expression

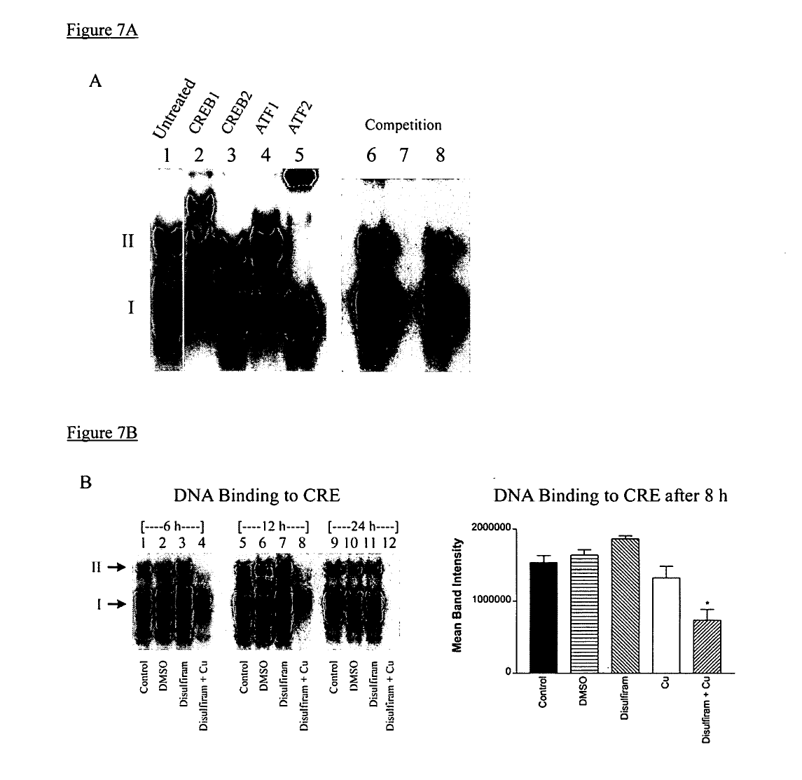

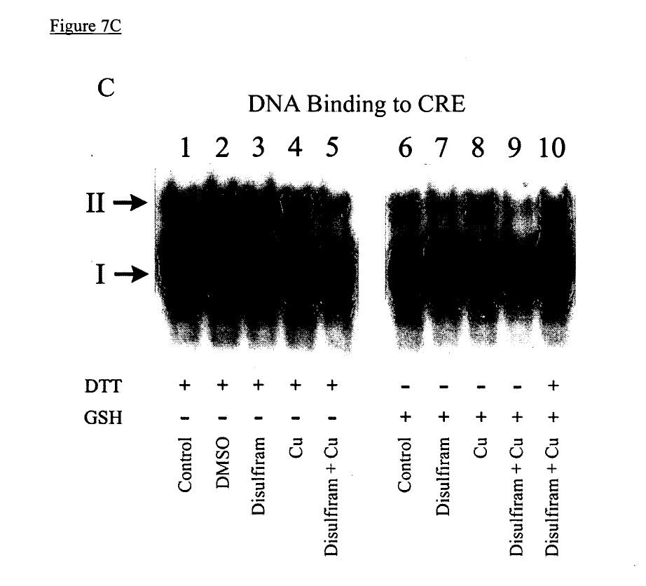

One critical location of cysteines is the DNA binding region of transcription factors, where sulfhydryls generally must remain reduced to insure effective transcription factor binding. (See, Klatt P, Molina E P, Lamas S. Nitric oxide inhibits c-Jun DNA binding by specifically targeted S-glutathionylation. J Biol Chem, 1999; 274:15857-15864). When cysteines in the positively-charged transcription factor DNA binding domain are oxidatively modified, repair processes are triggered that result in formation of mixed disulfides between glutathione and protein thiols. (See, Klatt P, Molina E P, Lamas S. Nitric oxide inhibits c-Jun DNA binding by specifically targeted S-glutathionylation. J Biol Chem, 1999; 274:15857-15864, Sies H. Glutathione and its role in cellular functions. Free Rad Biol Med, 1999; 27:916-921). Consequent to protein S-glutathionylation, the usually positively charged transcription factor DNA binding domain develops a negative charge imparted by the dual carboxylate end groups of glutathione, thereby repelling similarly-charged DNA and disrupting DNA-transcription factor binding. (See, Klatt P, Molina E P, Lamas S. Nitric oxide inhibits c-Jun DNA binding by specifically targeted S-glutathionylation. J Biol Chem, 1999; 274:15857-15864). The transcription factors NF-κB, activator protein-I (AP-1) and ATF/CREB all contain cysteines in their DNA binding regions as reactive sites for mixed disulfide formation. (See, Brar S S, Kennedy T P, Sturrock A B, et al. An NAD(P)H oxidase regulates growth and transcription in melanoma cells. Am J Physiol Cell Physiol, 2002; 282:C1212-C1224, Pineda-Molina E, Klatt P, Vazquez J, et al. Glutathionylation of the p50 subunit of NF-κB:a mechanism for redox-induced inhibition of DNA binding. Biochem, 2001; 40:14134-14142, Marshall H E, Stamler J S. Inhibition of NF-κB by S-nitrosylation. Biochem, 2001; 40:1688-1693, Nikitovic D, Holmgren A, Spyrou G. Inhibition of AP-1 DNA binding by nitric oxide involving conserved cysteine residues in Jun and Fos. Biochem Biophys Res Commun, 1998; 242:109-112, Goren I, Tavor E, Goldblum A, et al. Two cysteine residues in the DNA-binding domain of CREB control binding to CRE and CREB-mediated gene expression. J Mol Biol, 2001; 313:695-709, Richards J P, Bachinger H P, Goodman R H, et al. Analysis of the structural properties of cAMP-responsive element-binding protein (CREB) and phosphorylated CREB. J Biol Chem, 1996; 271:13716-13723). To determine if thiocarbamates might form mixed disulfides with these sulfhydryls, we studied DNA binding of the cyclic AMP response element CRE, which is of pivotal importance for melanoma proliferation. (See, Xie S, Price J E, Luca M, Jean D, et al. Dominant-negative CREB inhibits tumor growth and metastasis of human melanoma cells. Oncogene, 1997; 15:2069-2075, Jean D, Harbison M, McConkey D J, et al. CREB and its associated proteins act as survival factors for human melanoma cells. J Biol Chem, 1998; 273:24884-24890, Ronai Z, Yang Y-M, Fuchs S Y, et al. ATF2 confers radiation resistance to human melanoma cells. Oncogene, 1999; 16:523-531). Melanomas exhibited prominent constitutive DNA binding activity for CRE (FIG. 7A: CRL1619 melanoma cells exhibit constitutive DNA binding activity to the cyclic AMP response element (CRE) (lane 1). CRL1619 melanoma cells were grown to 60% confluence on 100×15 mm plastic Petri dishes, nuclear protein was harvested and electrophoretic mobility gel shift assays (EMSAs) were performed using the consensus oligonucleotides (5′-AGAGATTGCCTGACGTCAGAGAGCTAG-3′ and 3′-TCTCTAACGGACTGCAGTCTCTCGATC-5′) for the cyclic-AMP responsive element CRE, end-labeled by phosphorylation with [γ³²P]-ATP and T4 polynucleotide kinase. CRE complexes (I and II) are labeled. Supershift experiments performed by incubating the binding reaction with 1 μg of antibody before addition of labeled probe demonstrate that the upper complex II contains ATF-2 (lane 5), while the lower complex I is comprised primarily of CREB-1 (lane 2), with some ATF-1 (lane 4). Competition experiments shown in lanes 6-8 demonstrate specificity of the DNA binding reaction: Lane 6, untreated; lane 7, with 10× unlabeled CRE probe added to binding reaction; lane 8, with 10× unlabeled NF-κB probe added to binding reaction.), that was significantly reduced by treatment of cells with disulfiram and copper(II) (FIG. 7B: Treatment of melanoma cells with disulfiram and copper (II) inhibits transcription factor binding to CRE. CRL1619 melanoma cells were grown to 80% confluence, nuclear protein was harvested and EMSAs were performed for the cyclic-AMP responsive element CRE. Left: Treatment of cultures for 6, 12 or 24 hr with the combination of 5 μM disulfiram and 1.6 μM cupric sulfate substantially interrupted transcription factor binding to CRE. The ATF-2 containing complex II proved the more sensitive to inhibition. Right: EMSAs were performed using nuclear protein from replicate experiments (n=4) in which near confluent cells were treated for 8 h and densitometry was performed on the ATF-2 containing upper complex II. The combination of disulfiram plus copper(II) reduced DNA binding by half. *p<0.05 compared to other treatments.). Disulfiram and copper(II) also inhibited DNA binding of NF-κB. To determine if inhibition was from direct transcription factor modification, we added each agent directly to the binding reaction (FIG. 7C: The inhibitory effects of disulfiram or disulfiram plus copper(II) on transcription factor binding are potentiated in the presence of glutathione (GSH). EMSAs were performed with addition of disulfiram or disulfiram plus 1.6 μM CuSO₄ (Cu) directly to the binding reaction of nuclear protein and oligonucleotides. Disulfiram alone reduced DNA binding to CRE in the upper ATF2 containing complex II (lane 3). This was magnified when disulfiram was combined with copper(II) ions (lane 5). Results are consistent with modest disruption of ATF2 binding to CRE from formation of mixed disulfides between disulfiram and cysteines in the DNA binding region, and greater disruption when copper(II) is present to enhance mixed disulfide formation. However, reduction in CRE binding was much more pronounced when the binding reaction was performed with GSH instead of dithiothreitol (DTT) as the reducing agent [lane 7 for disulfiram, lane 9 for disulfiram plus copper(II)]. Inhibition of ATF2 containing complex II binding to CRE by disulfiram and copper(II) in the presence of GSH was reversed by simultaneous addition of the potent uncharged reducing agent DTT (lane 10).). Copper (II) facilitated inhibition of CRE DNA binding by disulfiram (lane 5), suggesting that metal ions might enhance formation of a mixed disulfide between the thiuram disulfide and cysteine sulfhydryls in the transcription factor DNA binding region. Synergistic inhibition of transcription factor DNA binding by copper(II) and disulfiram was even more pronounced when dithiothreitol was replaced by glutathione as the reducing agent in the binding buffer (lane 9). This suggests that glutathione, found in millimolar concentrations within the nucleus (see, Sies H. Glutathione and its role in cellular functions. Free Rad Biol Med, 1999; 27:916-921), might react with the mixed disulfide formed between the dithiocarbamate and protein cysteine sulfhydryls (see, Burkitt M J, Bishop H S, Milne L, et al. Dithiocarbamate toxicity toward thymocytes involves their copper-catalyzed conversion to thiuram disulfides, which oxidize glutathione in a redox cycle without the release of reactive oxygen species. Arch Biochem Biophys, 1998; 353:73-84), leading to a bulky, negatively-charged glutathione-containing mixed disulfide that can more effectively disrupt DNA binding. Disulfiram and copper(II) also reduced expression of cyclin A (FIG. 8: While disulfiram or copper(II) alone had little effect, treatment with the combination of disulfiram plus copper(II) reduced expression of cyclin A by over two-thirds at 24 hr, which would be expected to produce a site of cell cycle arrest consistent with that seen in FIG. 3. CRL1619 melanoma cells were plated at equal densities, grown to 80% confluence and in replicate experiments (n=4 each) treated with DMSO vehicle (lanes 1-4), 5 μM disulfiram (lanes 5-8), 1.6 μM CuSO₄ (Cu, lanes 9-12) or the combination of disulfiram and CuSO₄ (lanes 13-16). After 24 hr immunoblots were performed to assay for cyclin A. Quantitation of experiments by densitometry is shown below. *p<0.05 compared to all other treatments.), which is positively regulated by a CRE element (see, Desdoutets C, Matesic C G, Molina C A, et al. Cell cycle regulation of cyclin A gene expression by the cyclic AMP-responsive transcription factors CREB and CREM. Mol Cell Biol, 1995; 15:3301-3309), a phenomenon that would be expected to reduce cell cycle progression into G₂-M (FIG. 5). Disulfiram had no consistent effect on expression of cyclin B1, p21^{WAFI/CIPI}, p53 or bcl-2.

Disulfiram and Zinc(II) Inhibit Melanoma Growth and Angiogenesis in Mice

Melanoma cells transplanted into SCID mice grew rapidly as a spherical encapsulated mass. Tumor volume reached approximately 500 mm³ in controls by 16 days, when animals were sacrificed. Zinc(II) alone had no affect on tumor growth (FIG. 9: Adult female CB17-SCID mice (Harlan) were injected subcutaneously in the right groin with 5×10⁶ cells from a highly aggressive malignant human melanoma. Mice were fed either a standard diet containing 87 ppm zinc or a zinc supplemented diet (Harlan) containing 1,000 ppm zinc(II) as zinc acetate. On the day of tumor injection all mice began daily oral gavage of 0.2 ml of olive oil as a control or 0.2 ml of olive oil containing the indicated drug. Four groups were studied: Tumor Control (Con; n=10; 0.2 ml olive oil daily; standard zinc diet of 87 ppm); Zinc-Supplemented Control (Zn; n=10; 0.2 ml olive oil daily; zinc diet of 1,000 ppm); Disulfiram (DS; n=10; disulfiram 200 mg/kg/day in 0.2 ml olive oil; zinc diet of 87 ppm); and Zinc-Supplemented Diet+Disulfiram (DS+Zn; n=10; disulfiram 200 mg/kg/day in 0.2 ml olive oil; zinc diet of 1,000 ppm). When estimated tumor volume in controls approached 500 mm³, all mice were euthanized, and tumors were excised and weighed. Zinc(II) supplementation alone had no affect on tumor growth, but disulfiram alone and disulfiram plus zinc(II) supplementation all significantly inhibited tumor growth. *p<0.05 vs tumors in controls or Zn; ⁺p<0.001 versus tumors in controls or Zn.). However, treatment with disulfiram alone or disulfiram plus zinc(II) significantly inhibited tumor growth. In mice receiving disulfiram and a zinc(II)-enriched diet, tumors were less than a third (83±12 mg) of the size of tumors in either controls (289±57 mg) or in mice receiving a zinc-enriched diet alone (271±19 mg). Histologic sections of tumors from mice treated with disulfiram plus zinc demonstrated more cellular necrosis. There was also a significant reduction in the number of blood vessels per field in disulfiram or disulfiram plus zinc acetate treated mice, suggesting that thiocarbamates inhibit angiogenesis (vessels per field=5.8±0.8 for control; 5.4±1.6 for zinc-supplemented; 2.5±0.7 for disulfiram, p<0.05 vs. control; 2.0±0.7 for disulfiram+zinc, p<0.05 vs. control). Mice in all groups tolerated treatment well, although diarrhea was noted in animals receiving disulfiram plus a zinc(II)-enriched diet.

Case Report: Use of disulfiram and zinc(II) for treatment of metastatic melanoma in a patient.

The first use of disulfiram and zinc(II) to treat advanced Stage IV metastatic melanoma in a patient is reported herein. This was done with approval from the Carolinas Medical Center Institutional Review Board, informed consent was obtained, data was collected prospectively and the patient has been on no other treatment for melanoma. The subject treated was a 64 year-old woman who presented with a non-operable central liver metastasis from a T2 ocular melanoma that had been removed 5 years previously. She had developed abdominal pain and was found to have a 2.3 cm right hepatic metastasis and a 5.5 cm central liver metastasis confirmed as recurrent melanoma by biopsy. She declined chemotherapy, interleukin-2 therapy or liver perfusion. After granting informed consent, she was started on 250 mg disulfiram (Antabuse®, Wyeth) daily with the largest meal of the day. This dose was increased to 500 mg per day after a month. Zinc gluconate [50 mg chelated zinc(II), General Nutrition Center] was also given 3 times daily but not concurrent with disulfiram administration. This heavy metal and its dose were chosen for previously demonstrated safety in humans as the preventative treatment for Wilson's disease. Doses of each agent were those currently recommended for treatment of alcoholism and Wilson's disease, respectively. Upon starting the protocol, the patient suffered grade 1 (National Cancer Institute Common Toxicity Criteria, Version 2.0) diarrhea, nausea, depression, and malaise. Except for nausea, these side effects resolved within 2 months of continued treatment. Her abdominal pain also completely resolved and she returned to work. After 9 months, disulfiram was reduced to 250 mg per day, and her nausea ceased. She has continued on disulfiram 250 mg once and zinc gluconate 50 mg three times daily. All laboratory studies remained normal for an extended period of time. Repeated CT and PET scans after 3 months of therapy showed a >50% reduction in tumor size (FIG. 10 top). A PET scan 12 months after initiating treatment showed the lesions to be stable (FIG. 10 bottom), and the most recent CT scan after 42 months of treatment (FIG. 10 top, far right) showed that residual hepatic disease has remained stable. FIG. 10 shows the computed axial tomograms (CT, top) and positron emission spectrographs (PET, bottom) of athe 64 year old woman with Stage IV ocular melanoma metastatic to the liver. Before treatment, the patient had a 5.5 cm central liver metastasis, shown in both scans by a white arrow. After 3 months of treatment with disulfiram 500 mg daily and zinc gluconate 50 mg three times daily, the hepatic metastasis had decreased in volume by >50% in both scans (white arrows). After continuing treatment with 250 mg disulfiram daily and the same dose of zinc gluconate, the lesion remained stable in size at 10 and 14 months (white arrows). She continued to be clinically well and free of drug side effects on disulfiram and zinc gluconate for an extended period of time. After 53 continuous months of treatment with this regimen, the patient has experienced no quantifiable malignant progression. A follow-up abdominal CT scan after 42 months of therapy showed that the hepatic tumor burden had remained small. The patient remains clinically well and physically active after 53 continuous months of therapy.

Bis-Copper diethyldithiocarbamates have also been found to be efficacious on retarding the growth of human adenosquamous carcinoma of the lung and colon cancer as shown and described below.

Effect of Bis-Copper Diethyldithiocarbamate on Growth of H596 Human
Adenosquamous Carcinoma of the Lung

| | A₅₄₀ of MTT Formazan | | | | | |
|---|---|---|---|---|---|---|
| | 0 | 0.312 μM | 0.625 μM | 1.25 μM | 2.5 μM | 5.0 μM |
| 24 hr | .232 ± .026 | .146 ± .021 | .037 ± .006 | .013 ± .001 | .005 ± .001 | .011 ± .007 |
| 48 hr | .340 ± .018 | .158 ± .047 | .016 ± .008 | .011 ± .005 | .018 ± .016 | .000 ± .002 |
| 72 hr | .408 ± .030 | .038 ± .008 | .025 ± .007 | .019 ± .003 | .032 ± .005 | .057 ± .010 |
| 96 hr | .870 ± .107 | .063 ± .027 | .040 ± .017 | .021 ± .005 | .014 ± .004 | .006 ± .001 |

Cells grown in RPMI 1640 and stimulated with 10% fetal bovine serum (FBS) were plated at a density of 50,000 cells per well, and DMSO vehicle (5 μl per ml) or bis-copper diethyldithiocarbamate was added to wells at the indicated concentrations. After 24, 48, 72 or 96 hr, proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance-at 540 nm (A₅₄₀).

Effect of Bis-Copper Diethyldithiocarbamate on Growth of H596 Human
Adenosquamous Carcinoma of the Lung

| | A₅₄₀ of MTT Formazan | | | | | |
|---|---|---|---|---|---|---|
| | 0 | 25 nM | 75 nM | 125 nM | 375 nM | 625 nM |
| 24 hr | .384 ± .048 | .313 ± .020 | .327 ± .019 | .222 ± .022 | .170 ± .030 | .098 ± .015 |
| 48 hr | .244 ± .024 | .251 ± .026 | .209 ± .015 | .148 ± .010 | .088 ± .029 | .033 ± .008 |
| 72 hr | .308 ± .011 | .300 ± .042 | .260 ± .016 | .219 ± .021 | .099 ± .026 | .054 ± .007 |
| 96 hr | .808 ± .030 | .714 ± .074 | .672 ± .046 | .573 ± .036 | .410 ± .044 | .140 ± .070 |

Cells grown in RPMI 1640 and stimulated with 10% fetal bovine serum (FBS) were plated at a density of 50,000 cells per well, and DMSO vehicle (5 μl per ml) or bis-copper diethyldithiocarbamate was added to wells at the indicated concentrations. After 24, 48, 72 or 96 hr, proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance-at 540 nm (A₅₄₀).

Effect of Bis-Copper Diethyldithiocarbamate Complex on Growth of C26
Murine Colon Cancer Cells

| | A₅₄₀ of MTT Formazan | | | | | |
|---|---|---|---|---|---|---|
| | 0 | 25 nM | 75 nM | 125 nM | 375 nM | 625 nM |
| 24 hr | .165 ± .006 | .151 ± .020 | .152 ± .029 | .163 ± .039 | .121 ± .014 | .090 ± .006 |
| 48 hr | .247 ± .031 | .283 ± .021 | .229 ± .021 | .257 ± .023 | .119 ± .013 | .111 ± .026 |
| 72 hr | .411 ± .030 | .536 ± .044 | .415 ± .049 | .359 ± .018 | .246 ± .037 | .117 ± .024 |
| 96 hr | .643 ± .023 | .593 ± .033 | .437 ± .076 | .554 ± .056 | .406 ± .056 | .440 ± .062 |

Cells grown in RPMI 1640 and stimulated with 10% fetal bovine serum (FBS) were plated at a density of 50,000 cells per well, and DMSO vehicle (5 μl per ml) or bis-copper diethyldithiocarbamate was added to wells at the indicated concentrations. After 24, 48, 72 or 96 hr, proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance-at 540 nm (A₅₄₀).

Effect of Bis-Copper Diethyldithiocarbamate Complex on Growth of C38
Murine Colon Cancer Cells

| | A₅₄₀ of MTT Formazan | | | | | |
|---|---|---|---|---|---|---|
| | 0 | 25 nM | 75 nM | 125 nM | 375 nM | 625 nM |
| 24 hr | .070 ± .007 | .058 ± .011 | .087 ± .037 | .060 ± .009 | .057 ± .004 | .045 ± .002 |
| 48 hr | .099 ± .004 | .084 ± .008 | .085 ± .006 | .095 ± .005 | .035 ± .009 | .018 ± .003 |
| 72 hr | .138 ± .014 | .101 ± .012 | .111 ± .004 | .123 ± .008 | .033 ± .007 | .017 ± .002 |
| 96 hr | .563 ± .062 | .488 ± .044 | .473 ± .028 | .552 ± .039 | .286 ± .051 | .065 ± .017 |

Cells grown in RPMI 1640 and stimulated with 10% fetal bovine serum (FBS) were plated at a density of 50,000 cells per well, and DMSO vehicle (5 μl per ml) or bis-copper diethyldithiocarbamate was added to wells at the indicated concentrations. After 24, 48, 72 or 96 hr, proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance-at 540 nm (A₅₄₀).

TABLE 1
Effect of Complexation or Supplementation
of Copper Ions on Anti-Proliferative Activity of Disulfiram

| | |
|---|---|
| Treatment | % Growth Inhibition |

| | | |
|---|---|---|
| | HTB75 | HTB77 |
| | Ovarian Cancer | Ovarian Cancer |
| 0.5 μM Disulfiram | 75 ± 4 | 81 ± 2 |
| 0.5 μM Disulfiram + | 0 ± 4⁺ | 13 ± 5⁺ |
| 200 μM BCPS | | |
| 0.1 μM Disulfiram | 12 ± 4 | 5 ± 2 |
| 0.1 μM Disulfiram + | 75 ± 2⁺ | 83 ± 1⁺ |
| 0.8 μM CuSO₄ | | |
| | 520 | 596 |
| | Squamous | Adenosquamous |
| | Lung Cancer | Lung Cancer |
| 0.5 μM Disulfiram | 76 ± 3 | 69 ± 2 |
| 0.5 μM Disulfiram + | 0 ± 2⁺ | 5 ± 6⁺ |
| 200 μM BCPS | | |
| 0.25 μM Disulfiram | 66 ± 2 | 53 ± 4 |
| 0.25 μM Disulfiram + | 88 ± 2* | 91 ± 1⁺ |
| 0.8 μM CuSO₄ | | |

*p < 0.01 versus respective disulfiram concentration alone;
⁺p < 0.001 versus respective disulfiram concentration alone.

Cells stimulated with 10% fetal bovine serum (FBS) were plated at a density of 50,000 cells per well, and DMSO vehicle (5 μL per mL), disulfiram (DS) was added to wells at the indicated concentrations. To decrease the concentration of available Cu(II), the impermeate Cu(II) chelator bathocuproine disulfonic acid (BCPS) was added to medium. The increase available Cu(II), medium was supplemented with CuSO₄. After 48 hr proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance at 540 nm (A₅₄₀).

TABLE 2
DISULFIRAM IS ANTIPROLIFERATIVE FOR MALIGNANT CELLS

| | Mean Percent Inhibition of Growth | | | |
|---|---|---|---|---|
| | Concentration of Disulfiram (μM) | | | |
| Cell Line | 0.625 | 1.25 | 2.5 | 5.0 |
| Treatment initially | | | | |
| Melanoma M1585 | 100 ± 0^{A} | 100 ± 0^{A} | 100 ± 0^{A} | 100 ± 0^{A} |
| Prostate carcinoma | 6 ± 6^{A} | 29 ± 5^{A} | 48 ± 2^{A} | 86 ± 2^{A} |
| CRL 1435 (PC-3) | | | | |
| Squamous lung | 76 ± 3^{A} | 82 ± 4^{A} | 77 ± 4^{A} | 78 ± 3^{A} |
| carcinoma | | | | |
| NCI-H520 | | | | |
| Adenosquamous lung | 47 ± 4^{A} | 57 ± 4^{A} | 50 ± 3^{A} | 50 ± 4^{A} |
| carcinoma NCI-H596 | | | | |
| Small cell lung | 68 ± 3^{A} | 76 ± 6^{A} | 76 ± 5^{A} | 72 ± 3^{A} |
| carcinoma | | | | |
| NCI-H82 | | | | |
| Breast carcinoma | 69 ± 4^{A} | 94 ± 2^{A} | 100 ± 0^{A} | 100 ± 0^{A} |
| MDA-MB-453 | | | | |
| Treatment after 24 hours | | | | |
| Melanoma M1619 | 59 ± 4^{A} | 35 ± 4^{A} | 39 ± 3^{A} | 37 ± 4^{A} |
| Melanoma M1585 | 74 ± 4^{A} | 49 ± 7^{A} | 41 ± 2^{A} | 37 ± 6^{A} |
| Lung carcinoma | 30 ± 3^{A} | 30 ± 3^{A} | 29 ± 1^{A} | 34 ± 3^{A} |
| NCI-H596 | | | | |
| Breast carcinoma | 26 ± 5^{A} | 26 ± 2^{A} | 39 ± 2^{A} | 46 ± 4^{A} |
| MDA-MB-453 | | | | |

^{A}p < 0.01 compared to FBS + DMSO vehicle control

TABLE 3
DISULFIRAM POTENTIATES
THE ANTIPROLIFERATIVE
ACTIVITY OF CHEMOTHERAPEUTIC AGENTS

A540 of MTT Formazan

| A. Cisplatin (ng/mL) | DMSO vehicle | Disulfiram 2.5 μM |
|---|---|---|
| 0 | 1.433 ± 0.038 | |
| 1 | 1.739 ± 0.041 | 1.369 ± 0.033^{B} |
| 10 | 1.447 ± 0.047 | 1.221 ± 0.028 |
| 100 | 1.372 ± 0.052 | 1.183 ± 0.038^{A} |
| 1,000 | 1.381 ± 0.098 | 0.921 ± 0.027^{A} |
| B. Carmustine (μM) | DMSO vehicle | Disulfiram 0.6 μM |
| 0 | 0.104 ± 0.010 | |
| 1 | 0.197 ± 0.004 | 0.042 ± 0.003^{C} |
| 10 | 0.152 ± 0.011 | 0.025 ± 0.002^{C} |
| 100 | 0.020 ± 0.002 | 0.030 ± 0.023 |
| 1,000 | 0.003 ± 0.000 | 0.004 ± 0.000 |

^{A}p < 0.05 compared to DMSO vehicle;
^{B}p < 0.01 compared to DMSO vehicle;
^{C}p < 0.001 compared to DMSO vehicle

TABLE 4
EFFECT OF DISULFIRAM (DS) ON INTRACELLULAR COPPER

| Treatment | Copper (ng/mL) |
|---|---|
| 10% FBS | 56 ± 7 |
| FBS ± DMSO | 52 ± 4 |
| FBS ± 0.625 μM DS | 76 ± 11 |
| FBS ± 1.25 μM DS | 102 ± 5^{A} |
| FBS ± 2.5 μM DS | 160 ± 17^{A} |
| FBS ± 5.0 μM DS | 195 ± 3^{B} |

^{A}p < 0.01 compared to DMSO control;
^{B}p < 0.001 compared to DMSO control.

TABLE 5
Data Corresponding to FIG. 1

| | | 1619 disulfiram growth curves | | | | | | | | | |
|---|---|---|---|---|---|---|---|---|---|---|---|
| | | X Values | | | | | | | | | |
| | | A | | B | | C | | D | | E | |
| | | X Title | | | | | | | | | |
| | | 0 | | 0.1 | | 0.25 | | 0.5 | | 5.0 | |
| | X | Y | SEM | Y | SEM | Y | SEM | Y | SEM | Y | SEM |
| 1 | 24.0 | 0.2490 | 0.00850 | 0.250 | 0.010 | 0.2080 | 0.0094 | 0.140 | 0.00650 | 0.0250 | 0.0006 |
| 2 | 48.0 | 0.9200 | 0.05640 | 0.915 | 0.047 | 0.6790 | 0.0340 | 0.339 | 0.03820 | 0.0640 | 0.0125 |
| 3 | 72.0 | 1.6430 | 0.09090 | 1.504 | 0.092 | 1.2500 | 0.0970 | 0.669 | 0.06470 | 0.0600 | 0.0214 |
| 4 | 96.0 | 2.0000 | 0.19580 | 1.832 | 0.180 | 1.5360 | 0.1242 | 1.084 | 0.07310 | 0.0100 | 0.0020 |

TABLE 6
Data Corresponding to FIG. 3

| | | 1619 disulfiramBCPS | | | | | | | |
|---|---|---|---|---|---|---|---|---|---|
| | | X Values | | | | | | | |
| | | A | | B | | C | | D | |
| | | X Title | | | | | | | |
| | | Control | | DS | | BCPS + DS | | BCPS | |
| | X | Y | SEM | Y | SEM | Y | SEM | Y | SEM |
| 1 | | 1.3450 | 0.030 | 0.6940 | 0.0290 | 1.20 | 0.0250 | 1.3030 | 0.0470 |

TABLE 7
Data Corresponding to FIG. 9

| | | X Values | | | | | | | |
|---|---|---|---|---|---|---|---|---|---|
| | | A | | B | | C | | D | |
| | | X Title | | | | | | | |
| | | | | | | Di- | | Di- | |
| | | Control | | Zn | | sulfiram | | sulfiram + Zn | |
| | X | Y | SEM | Y | SEM | Y | SEM | Y | SEM |
| 1 | | 289.0 | 57.0 | 271.0 | 19.0 | 190.0 | 26.0 | 83.0 | 12.0 |

Preparation of Metal Compounds

### EXAMPLE 1

### Preparation of Dichloro(diethyldithiocarbamato)gold(III) from Disulfiram and Tetrachloroauric Acid

Disulfiram (79.4 mg, 0.268 mmol) was dissolved in chloroform (10 mL) and placed in a 50 mL screw cap test tube. An aqueous solution of tetrachloroauric acid trihydrate (493.5 mg, 1.253 mmol in 15 mL water) was added to the chloroform solution. The resulting solution was vigorously mixed for five minutes. The contents of the test tube were transferred to a 30 mL test tube and the two layers separated by centrifuge. The aqueous layer was discarded and the chloroform was allowed to evaporate in a petri dish resulting in long, dark, orange-brown needles. The product was recrystallized from chloroform/acetonitrile and the final product identified to be [AuCl₂(DEDTC)] by X-ray crystallography. The structure [AuCl₂(DEDTC)] is shown below:

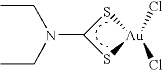

### EXAMPLE 2

### Preparation of Dichloro(diethyldithiocarbamato)gold(III) from Diethylammonium Diethyldithiocarbamate and Tetrachloroauric Acid

Diethylammonium diethyldithiocarbamate (449.2 mg, 2.020 mmol) was dissolved in water (10 mL). Tetrachloroauric acid trihydrate (775.5 mg, 1.969 mmol) was dissolved in water (10 mL). The aqueous solution of diethylammonium diethyldithiocarbamate was added to the aqueous gold(III) solution and the resulting mixture shaken for 2-3 minutes and allowed to settle. A bright yellow precipitate formed, which was separated by means of centrifuging for 10 minutes. The water was decanted and the solid separated by filtration through Whatman #2 filter paper. The precipitate was washed with water and dissolved in chloroform. Any particulate matter in the solution was removed by filtration through 0.45-μm polytetrafluoroethylene (PTFE). The resulting solution was placed in a petri dish for recrystallization. The product was characterized by means of X-ray crystallography, which indicated [AuCl₂(DEDTC)] was formed.

### EXAMPLE 3

### Preparation of Bis(diethyldithiocarbamato)copper(II) from Diethylammonium Diethyldithiocarbamate and Copper(II) Chloride

An aqueous solution of diethylammonium diethyldithiocarbamate (450.4 mg, 2.025 mmol in 15 mL of water) was added drop-wise to an aqueous solution of copper(II) chloride dihydrate (359.5 mg, 2.109 mmol in 15 mL water) with manual stirring. A dark brown precipitate formed and the reaction mixture was shaken for 5 minutes. The precipitate was separated by filtration and washed with water. The filtrate was dissolved in chloroform and any particulate matter was removed by filtration through 0.45 μm PTFE and a portion placed in a petri dish to enable crystallization. The remaining filtrate was stored in a beaker. Crystals formed and the product, bis(diethyldithiocarbamato)copper(II), [Cu(DEDTC)₂], was characterized by X-ray crystallography.

### EXAMPLE 4

### Preparation of Tris(diethyldithiocarbamato)gallium(III) from Ammonium Diethyldithiocarbamate and Gallium Nitrate

An aqueous solution of ammonium diethyldithiocarbamate (670.62 mg, 4.0323 mmol) was combined with an aqueous solution of gallium nitrate hydrate (518.72 mg) and a white precipitate was observed to form. The precipitate was separated by filtration, rinsed with water and dried in an oven. The product was dissolved in chloroform for recrystallization. The resulting crystals were characterized as tris(diethyldithiocarbamato)gallium(III), [Ga(DEDTC)₃], by means of X-ray crystallography.

### EXAMPLE 5

### Preparation of Ammine(diethyldithiocarbamato)nitroplatinum(II) from Diammineplatinum(II) Nitrite and Sodium Diethyldithiocarbamate

Diammineplatinum(II) nitrite in ammonium hydroxide (5.0 wt % as platinum, 8.5893 g, 2.202 mmol) was placed in a Schlenck flask. Sodium diethyldithiocarbamate (0.5677 g, 2.520 mmol) in a sufficient amount of water was added to the diammineplatinum(II) nitrite solution. A cloudy, light blue color was observed and a precipitate began to form with stirring. The solution was stirred for 1.5 hours. A yellow precipitate was evident and the contents of the flask were transferred to a separatory funnel. The product was extracted with an appropriate amount of dichloromethane. After separation, the dichloromethane was removed by rotary evaporation. The resulting yellow solid was dissolved in an appropriate amount of dichloromethane and particulate matter removed by filtration through 0.45-μm polytetraflouroethylene (PTFE). The supernatant was placed in a small beaker and a small amount of diethyl ether was added. The beaker was placed in a nitrogen cabinet for recrystallization. The resulting crystalline product was characterized by single crystal X-ray crystallography as ammine(diethyldithiocarbamato)nitroplatinum(II), [Pt(NO₂)(NH₃)(DEDTC)].

### EXAMPLE 6

### Preparation of Bis(diethyldithiocarbamato)platinum(II) from cis-Dichlorodiammineplatinum(II) and Sodium Diethyldithiocarbamate

cis-Dichlorodiammine platinum(II) (0.4758 g, 1.586 mmol) was placed in a round bottom flask with an appropriate amount of water. A small amount of methanol was added to aid in dissolution. Sodium diethyldithiocarbamate (0.38725 g, 1.719 mmol) was dissolved in an appropriate amount of methanol and added to the cis-dichlorodiammineplatinum(II) solution. After several hours of stirring, dichloromethane was added and the product was extracted. The dichloromethane was removed by rotary evaporation and the green product dissolved in a small amount of dichloromethane with particulate matter removed by filtration through 0.45-μm polytetraflouroethylene (PTFE). Diethyl ether was added to the solution and the flask placed in a nitrogen cabinet to form crystals. Dark yellow crystals formed and product bis(diethyldithiocarbamato)platinum(II), [Pt(DEDTC)₂], was characterized by X-ray crystallography.

### EXAMPLE 7

### Preparation of Tris(diethyldithiocarbamato)manganese(III) from Manganese(II) Chloride and Sodium Diethyldithiocarbamate

Manganese(II) chloride (0.5501 g, 4.371 mmol) was dissolved in an appropriate amount of water. Sodium diethyldithiocarbamate (2.4144 g, 10.716 mmol) was dissolved in an appropriate amount of water and the two solutions were combined. A brown precipitate formed immediately and, after stirring, the contents of the flask were transferred to a separatory funnel. The product was extracted with dichloromethane until the dichloromethane layer was light burgundy. The organic layers were combined, washed with water, separated and the dichloromethane was removed by rotary evaporation. A portion of the resulting dark solid was dissolved in an appropriate amount of dichloromethane and particulate matter was removed by filtration through 0.45-μm polytetrafluoroethylene (PTFE). The supernatant was placed in a small beaker and a small amount of diethyl ether was added. The beaker was placed in a nitrogen cabinet for recrystallization. The resulting crystalline product was characterized by single crystal X-ray crystallography as tris(diethyldithiocarbamato)manganese(III), [Mn(DEDTC)₃].

### EXAMPLE 8

### Preparation of Tris(diethyldithiocarbamato)iron(III) from Iron(III) Nitrate and Ammonium Diethyldithiocarbamate

Iron(III) nitrate nonahydrate (796.97 mg, 1.973 mmol) was dissolved in an appropriate amount of water. Ammonium diethyldithiocarbamate (679.93 mg, 4.088 mmol) was dissolved in an appropriate amount of water and the two solutions were combined. A black precipitate formed and the product was removed by filtration through Whatman #4 filter paper. The product was dried in vacuo for approximately one hour and then dissolved in an appropriate amount of chloroform to recrystallize. The resulting crystalline product was characterized by single crystal X-ray crystallography as tris(diethyldithiocarbamato)iron(III), [Fe(DEDTC)₃].

### EXAMPLE 9

### Preparation of Dibromo(diethyldithiocarbamato)gold(III) from Tetrabromoauric Acid and Sodium Diethyldithiocarbamate

Tetrabromoauric acid (0.6406 g, 0.4318 mmol) was dissolved in a solution of dichloromethane (10 mL) and absolute ethanol (20 mL). Sodium diethyldithiocarbamate (0.2168 g, 0.9622 mmol) was dissolved in absolute ethanol (50 mL) and the two solutions were combined with vigorous stirring. An orange-brown solution with some precipitate was observed. Additional sodium diethyldithiocarbamate (0.4076 g, 1.809 mmol) was added to the solution, dissolved, and then the solution was transferred to a separatory funnel after any particulate matter was removed by filtration through Whatman #4 filter paper. The organic phase was extracted with water using multiple washings until the aqueous phase was almost colorless. The aqueous layers were combined and the water removed from the aqueous phase by rotary evaporation. The resulting product was recrystallized from acetonitrile/diethyl ether in a nitrogen cabinet. The resulting crystalline product was characterized by single crystal X-ray as dibromo(diethyldithiocarbamato)gold(III), [AuBr₂(DEDTC)].

Many modification and other embodiments will come to mind to one skilled in the art to which this invention pertains, having the benefit of the teachings presented in the descriptions and the associated drawings contained herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method of treating cancer in animals comprising administering to an animal in need of such treatment a therapeutically effective amount of at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different; and
wherein each A, B and C may be the same or different.

2. A method according to claim 1, wherein R¹ and R² at each occurrence are independently selected from the group consisting hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocyclyl, heterocycloalkyl, aryl, and heteroaryl.

3. A method according to claim 1, wherein R¹ and R² at each occurrence are independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl.

4. A method according to claim 3, wherein R¹ and R² are independently selected from the group consisting of C₁-C₆ alkyl, C₁-C₆ alkoxy, C₂-C₆ alkenyl with one to three double bonds, C₂-C₆ alkynyl with one or two triple bonds, C₃-C₈ cycloalkyl, aryl, heteroaryl, heterocyclyl, heterocycloalkyl and heterocyclyl.

5. A method according to claim 3, wherein the C₁-C₆ alkyl group is methyl, ethyl, propyl, isopropyl, n-butyl, sec-butyl, tert-butyl, pentyl, 2-pentyl, isopentyl, neopentyl, hexyl, 2-hexyl, 3-hexyl or 3-methylpentyl.

6. A method according to claim 3, wherein the C₁-C₆ alkoxy group is methoxy, ethoxy, propoxy, isopropoxy, n-butoxy, sec-butoxy, tert-butoxy, pentoxy, isopentoxy, neopentoxy, hexoxy or 3-methylpentoxy.

7. A method according to claim 3, wherein the C₂-C₆ alkenyl group is ethenyl, propenyl, 1-but-3-enyl, 1-pent-3-enyl or 1-hex-5-enyl.

8. A method according to claim 3, wherein the C₂-C₆ alkynyl group is ethynyl, propynyl, butynyl or pentyn-2-yl.

9. A method according to claim 3, wherein the cycloalkyl group is cyclopropyl, cyclobutyl, cyclopentyl or cyclohexyl.

10. A method according to claim 3, wherein the aryl group is phenyl, 1-naphthyl, 2-naphthyl, indanyl, indenyl, dihydronaphthyl, tetralinyl or 6,7,8,9-tetrahydro-5H-benzo[α]cycloheptenyl.

11. A method according to claim 3, wherein the heteroaryl group is pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide or benzothiopyranyl S,S-dioxide.

12. A method according to claim 3, wherein the heterocycloalkyl or heterocyclyl is a carbocyclic ring system of 4-, 5-, 6-, or 7-membered rings which includes fused ring systems of 9-11 atoms containing at least one and up to four heteroatoms selected from nitrogen, oxygen, or sulfur.

13. A method according to claim 3, wherein the heterocycloalkyl or heterocyclyl group is morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide or homothiomorpholinyl S-oxide.

14. A method according to claim 1, wherein R¹ and R² are ethyl.

15. A method according to claim 1, wherein M is a main group metal, a transition metal, a lanthanide or an actinide.

16. A method according to claim 14, wherein M is selected from the group consisting of arsenic, bismuth, gallium, manganese, selenium, zinc, titanium, vanadium, chromium, iron, cobalt, nickel, copper, silver, platinum(II) and gold.

17. A method according to claim 16, wherein M is gold(III) or copper(II).

18. A method according to claim 17, wherein M is copper(II).

19. A method according to claim 1, wherein A is an anionic ligand selected from the group consisting of Cl⁻, Br⁻, F⁻, I⁻, NO₂⁻, ⁻OR³, ⁻SR³, ⁻N(R³)₂ and ³¹ P(R³)₂, or a mixture thereof, wherein R³ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl.

20. A method according to claim 19, wherein R³ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl.

21. A method according to claim 19, wherein R³ is independently hydrogen, methyl, ethyl, isopropyl, tert-butyl, or phenyl.

22. A method according to claim 1, wherein A is an organic-based anionic ligand selected from the group consisting of acetate, formate, oxalate, tartrate and lactate, or a mixture thereof.

23. A method according to claim 1, wherein A is an anionic ligand selected from the group consisting of Cl⁻, Br⁻, F⁻ and I⁻, or a mixture thereof.

24. A method according to claim 1, wherein each B ligand is a neutral ligand independently selected from the group consisting of NH₃, (R⁴)₂O, N(R⁴)₃, P(R⁴)₃ and (R⁴)₂S, or a mixture thereof, wherein R⁴ is independently hydrogen, C₁₋₁₂ alkyl, C₂₋₁₂ alkenyl, C₂₋₁₂ alkynyl, C₃₋₈ cycloalkyl, C₃₋₈ cycloalkenyl, C₅₋₈ cycloalkynyl, heterocycyl, aryl, or heteroaryl.

25. A method according to claim 24, wherein R⁴ is independently selected from the group consisting of C₁-C₁₀ alkyl, C₁-C₁₀ alkoxy, C₂-C₁₀ alkenyl with one to three double bonds, C₂-C₁₀ alkynyl with one or two triple bonds, C₃-C₁₀ cycloalkyl, aryl, heteroaryl, heterocycloalkyl and heterocyclyl.

26. A method according to claim 25, wherein R⁴ is independently H, methyl, ethyl, isopropyl, tert-butyl, or phenyl.

27. A method according to claim 1, wherein C is NO⁺ or NO₂⁺.

28. A method according to claim 1, wherein the (S₂CNR¹R²) portion of the neutral compound is bound to the metal ion through both sulfur atoms.

29. A method according to claim 1, wherein M has a coordination number of two.

30. A method according to claim 29, wherein the neutral compound is of the formulae: 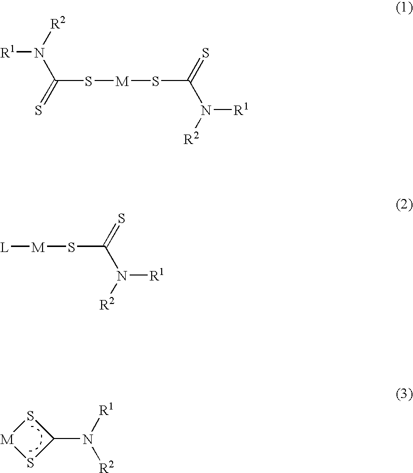
wherein L is a ligand selected from A, B or C.

31. A method according to claim 1, wherein M has a coordination number of three.

32. A method according to claim 31, wherein the neutral compound is of the formulae: 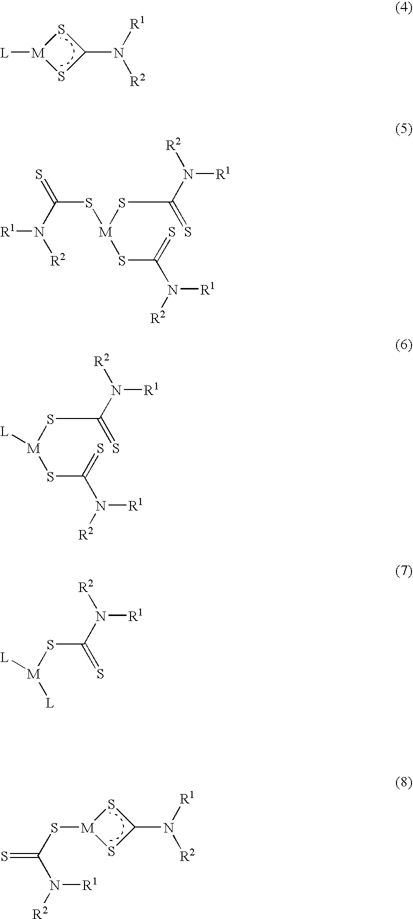
wherein L is ligand independently selected from A, B or C.

33. A method according to claim 1, wherein M has a coordination number of four.

34. A method according to claim 33, wherein the neutral compound is of the formulae: 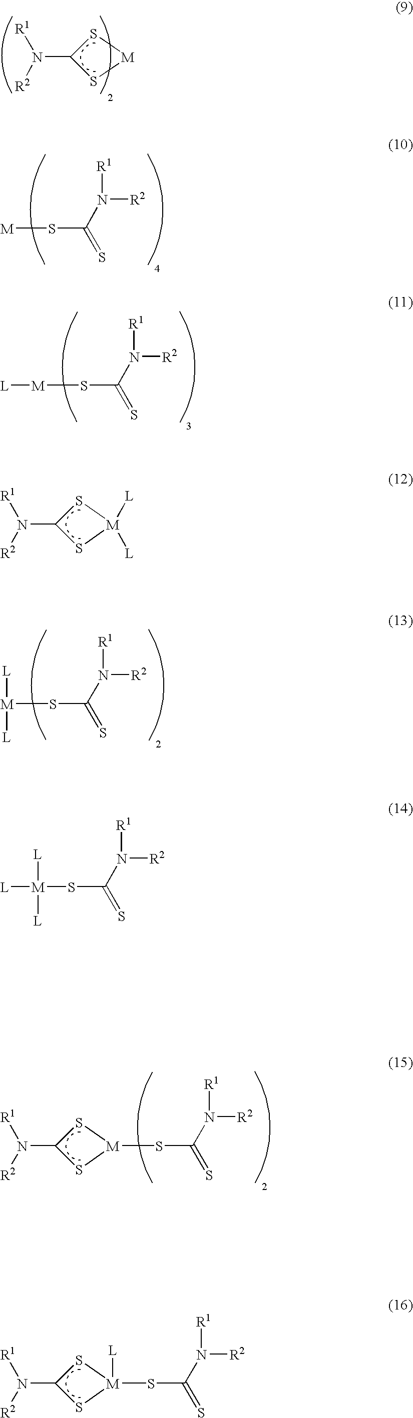
wherein L is a ligand independently selected from A, B or C.

35. A method according to claim 1, wherein M has a coordination number of five.

36. A method according to claim 35, wherein the neutral compound is of the formulae: 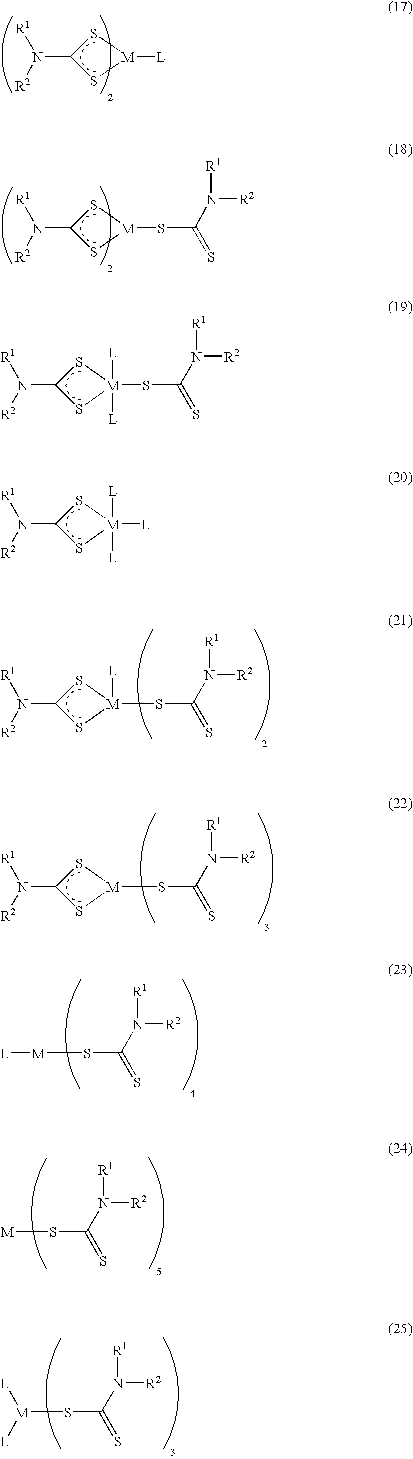 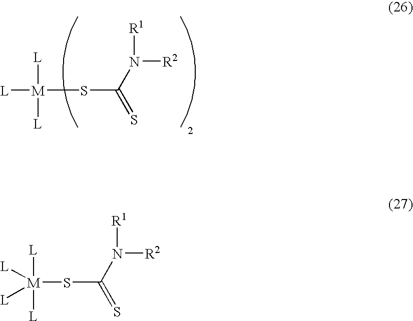
wherein L is ligand independently selected from A, B or C.

37. A method according to claim 1, wherein M has a coordination number of six.

38. A method according to claim 37, wherein the neutral compound is of the formulae: 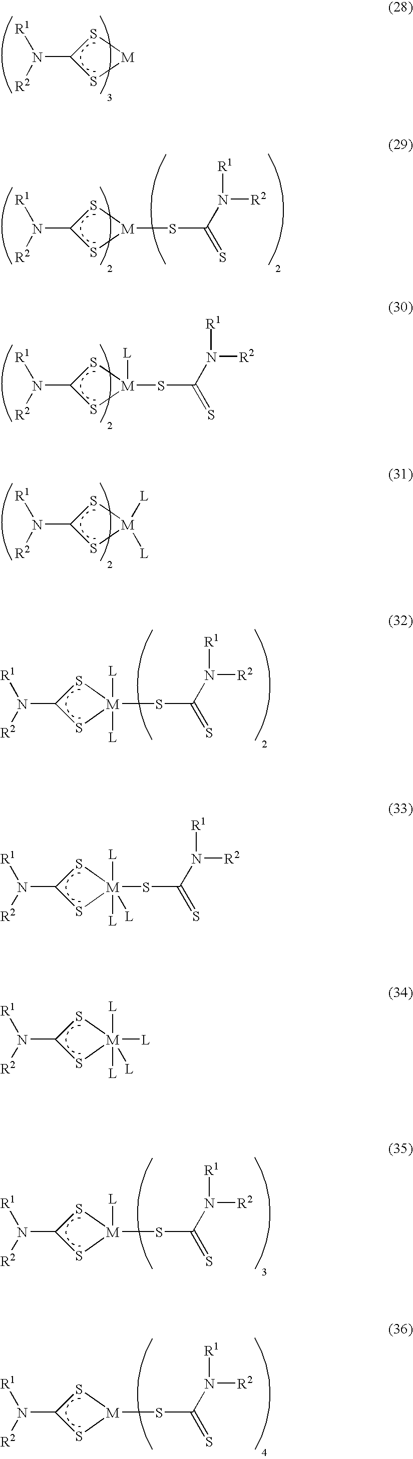 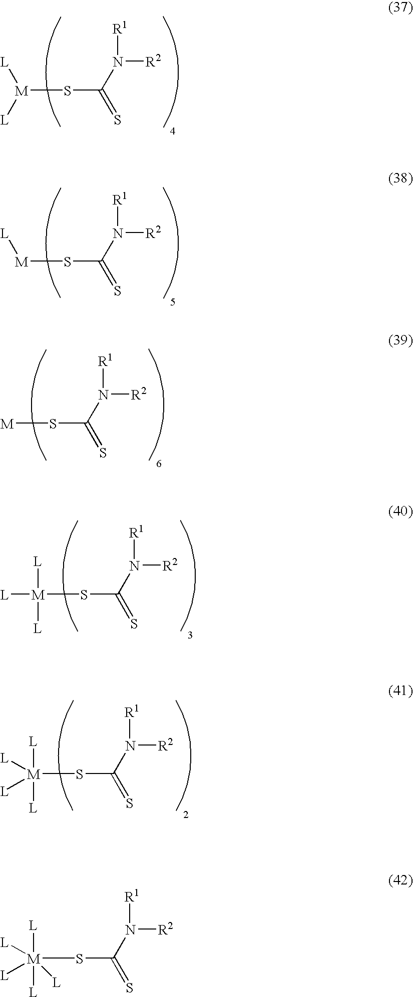
wherein L is ligand independently selected from A, B or C.

39. A method according to claim 1, wherein the neutral compound is of the formula:

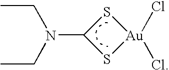

40. A method according to claim 1, wherein the neutral compound is of the formula:

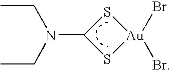

41. A method according to claim 1, wherein the neutral compound is of the formula:

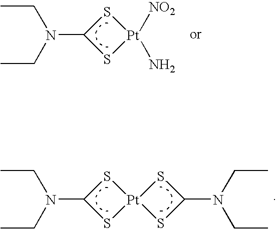

42. A method according to claim 1, wherein the neutral compound is of the formula:

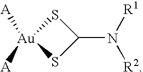

43. A method according to claim 42, wherein each A is independently a ligand selected from the group consisting of Cl⁻, Br³¹ , F⁻, I⁻ and NO₂⁻.

44. A method according to claim 43, where the neutral compound is of the formula:

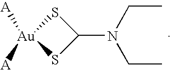

45. A method according to claim 1, wherein the (S₂CNR¹R²) portion of the neutral compound is of the formula: 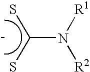
and is bound to M through both sulfur atoms.

46. A method according to claim 1, wherein the animal is a mammal.

47. A method according to claim 46, wherein the mammal is a human.

48. A method according to claim 47, wherein the therapeutically effective amount is administered in a dosage of between about 1 mg to about 1000 mg per day.

49. A method according to claim 48, wherein the therapeutically effective amount comprises a dosage of between about 25 mg to about 500 mg per day.

50. A method according to claim 47, wherein the therapeutically effective amount of the neutral compound is administered parenterally.

51. A method according to claim 47, wherein the therapeutically effective amount of the neutral compound is administered orally.

52. A method according to claim 1, where the cancer is selected from the group consisting of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate cancer, adenocarcinoma of the colon and nodal or hepatic metastases, or a combination thereof.

53. A method according to claim 1, where the cancer is selected from the group consisting of melanoma, lung cancer, breast cancer, colon and prostate cancer, or a combination thereof.

54. A method of treating cancer in animals comprising administering to an animal in need of such treatment a therapeutically effective amount of a pharmaceutical formulation comprising at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different;
wherein each A, B and C may be the same or different; and
a pharmaceutically acceptable excipient, diluent, solubilizer, solvent, adjuvant or carrier, or a mixture thereof.

55. A method of sensitizing cancerous tumors to conventional cancer chemotherapy or radiation therapy comprising administering to an animal with such tumors and in need of such treatment a therapeutically effective amount of at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge; and
wherein each (S₂CNR¹R²) portion of the compound is bound to the metal ion through one or both sulfur atoms;
wherein each R¹ and R² may be the same or different; and
wherein each A, B and C may be the same or different.

56. A method of sensitizing cancerous tumors to conventional cancer chemotherapy or radiation therapy comprising administering to an animal with such tumors and in need of such treatment a therapeutically effective amount of a pharmaceutical formulation comprising at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different;
wherein each A, B and C may be the same or different; and
a pharmaceutically acceptable excipient, diluent, solubilizer, solvent, adjuvant or carrier, or a mixture thereof.

57. A method of potentiating cancerous tumors to conventional cancer chemotherapy or radiation therapy comprising administering to an animal with such tumors and in need of such treatment a therapeutically effective amount of at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge; and
wherein each (S₂CNR¹R²) portion of the compound is bound to the metal ion through one or both sulfur atoms;
wherein each R¹ and R² may be the same or different; and
wherein each A, B and C may be the same or different.

58. A method of potentiating cancerous tumors to conventional cancer chemotherapy or radiation therapy comprising administering to an animal with such tumors and in need of such treatment a therapeutically effective amount of a pharmaceutical formulation comprising at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different;
wherein each A, B and C may be the same or different; and
a pharmaceutically acceptable excipient, diluent, solubilizer, solvent, adjuvant or carrier, or a mixture thereof.

59. A method according to claim 1, wherein the cancer is a multidrug-resistant.

60. A method according to claim 54, wherein the cancer is a multidrug-resistant.

61. A method for treating cancer in an animal, and for treating, removing or preventing multi-drug resistance in the animal, comprising administering to an animal in need of such treatment a therapeutically effective amount of at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R²) portion of the compound is bound to the metal ion through one or both sulfur atoms;
wherein each R¹ and R² may be the same or different; and
wherein each A, B and C may be the same or different.

62. A method for treating cancer in an animal, and for treating, removing or preventing multi-drug resistance in the animal, comprising administering to the animal in need of such treatment, a therapeutically effective amount of a pharmaceutical formulation comprising at least one neutral compound of the formula (I):
[AₓB_{y}C_{z}M(S₂CNR¹R²)ₙ] (I)
wherein
R¹ and R² at each occurrence are independently hydrogen, substituted or unsubstituted alkyl, cycloalkyl, heteroalkyl, alkoxy, alkenyl, alkynyl, aryl, heteroaryl, heterocyclyl or heterocycloalkyl;
M is a metal ion;
each A is independently an anionic ligand;
each B is independently a neutral ligand;
each C is independently a cationic ligand;
n is an integer from 1-10, where when n is greater than 1, each (S₂CNR¹R²) may be the same or different;
x, y and z are independently 0 or integers from 1-8;
wherein the coordination number of M is an integer of 1-10;
wherein the oxidation state of M is an integer of −1 to +8;
wherein n, x, y and z are selected such that the coordination number and the oxidation state of the metal ion are satisfied;
wherein the compound has an overall neutral charge;
wherein each (S₂CNR¹R 2) portion of the compound is bound to the metal ion through one or both sulfur atoms;
wherein each R¹ and R² may be the same or different;
wherein each A, B and C may be the same or different; and
a pharmaceutically acceptable excipient, diluent, solubilizer, solvent, adjuvant or carrier, or a mixture thereof.


SEQUENCE LISTING (4 sequences)

SEQ ID NO: 1 - DNA, 27 bp, Artificial
  Consensus oligonucleotide for the cyclic-AMP responsive element CRE.
agagattgcctgacgtcagagagctag

SEQ ID NO: 2 - DNA, 27 bp, Artificial
  Consensus oligonucleotide for the cyclic-AMP responsive element CRE.
tctctaacggactgcagtctctcgatc

SEQ ID NO: 3 - DNA, 22 bp, Artificial
  Consensus oligonucleotide for NF- B (p50).
agttgaggggactttcccaggc

SEQ ID NO: 4 - DNA, 22 bp, Artificial
  Consensus oligonucleotide for NF- B (p50).
tcaactcccctgaaagggtccg